(19) United States
(12) Patent Application Publication
(10) Pub. No.: US 2017/0226158 A1
(43) Pub. Date: Aug. 10, 2017

(54) INTRACELLULAR ANTIGEN BINDING

(71) Applicant: MedImmune Limited, Cambridge (GB)

(72) Inventors: Ralph MINTER, Cambridge (GB); Ron JACKSON, Cambridge (GB); Lasse KJELLERUP, Kastrup (DK)

(21) Appl. No.: 15/503,059

(22) PCT Filed: Aug. 11, 2015
(86) PCT No.: PCT/EP2015/068451

§ 371 Date: Feb. 10, 2017

Related U.S. Application Data
(60) Provisional application No. 62/035,793, filed on Aug. 11, 2014.

(51) Int. Cl. C07K 14/00; C12N 15/10

(52) U.S. Cl. CPC C07K 14/001; C12N 15/1037; C07K 2318/20

(57) ABSTRACT

The disclosure generally provides a Designed Ankyrin Repeat Protein (DARPin) that specifically binds to an antigen, the DARPin having an N-terminal cap section, at least two Ankyrin Repeat (AR) module sections, and a C-terminal cap section, characterized in that the DARPin has a charge that is less negative than known 5 DARPins. The disclosure also provides for generation of a library of the less negative DARPins.

### FIELD OF THE INVENTION

The present invention relates to intracellular delivery of molecules capable of binding to an antigen. More specifically, the present invention relates to a Designed Ankyrin Repeat Protein (DARPin) capable of internalising into a cell. A DARPin of the invention may bind a target antigen including but not limited to, target antigens present on a cell surface, extracellular antigens, and intracellular target antigens. DARPins of the invention may be capable of binding to an intracellular target antigen. The present invention also relates to methods of making such DARPins.

### BACKGROUND

DARPins are binding molecules which can be selected to bind a variety of targets. This antibody-like ability gives the protein some of the same functions as antibodies. DARPins have several properties that make them as attractive as antibodies. They are relatively small, with a molecular weight of −17 KiloDaltons (KDa). This is about eight times lower than Immunoglobulin G antibodies. DARPins are very stable proteins and have melting temperatures (Tm) values ranging from 66 to above 85° C. (Binz et al, 2003). DARPins can have high affinity to their antigens with dissociation (KD) values down to picomolar ranges (Zahnd et al, 2007). The study of DARPins is also greatly facilitated by their relatively easy production as they can be expressed in high amounts with up to 200 mg/l protein in simple shake flask culture in the low expressor strain XL1-blue (Binz et al, 2003). Additionally, DARPins are well suited to high throughput selection methods, such as ribosome and phage display, which allows several billion variants to be tested against a given antigen.

DARPin antibodies are a relatively new drug format and only limited testing has been done. Only one DARPin has so far entered clinical development: the anti-VEGF-A DARPin ‘MP0112’ has completed two phase I trials against two different diseases, and are currently in phase II (ClinicalTrials.gov Identifier: NCT01397409). The phase I studies were performed with a single injection in the eye of patients, and proved safe and well tolerated (Wurch et al., 2012).

DARPins are derived from natural ankyrin repeat proteins. The biological function of ankyrins is to mediate numerous key protein-protein interactions (Li et al, 2006). The ankyrins consists of repeat sections, which are stacked together to form a rigid protein domain (Li et al, 2006). These ankyrin repeat sections have been designed in a consensus approach to form one ankyrin repeat (AR) module (Binz et al, 2003).

The repeat sections each contain seven variable positions and with three AR's the theoretical diversity reaches 10²³ (Binz et al, 2003). The repeats are flanked with an N- and C-Cap, that function to seal the hydrophobic core of the stack of AR's. They are essential for efficient folding in the cell and for avoiding aggregation (Interlandi et al, 2008). The N- and C-Cap have many negatively charged residues, which are important for the stability (Interlandi et al, 2008): the framework positions alone contain a theoretical net charge of −14, which may explain their many favourable properties. Overall the theoretical net charge of a DARPin tends to be around −12 to −16. This large negative charge can limit binding to certain targets and limits the use of DARPins in applications where a large negative net charge is undesirable. Thus, there is a need for DARPins have a smaller negative net charge, a net neutral charge or as detailed further below, a positive net charge.

Most, if not all, therapeutic binding molecules such as DARPins and antibodies are limited to extracellular target antigens, since access to intracellular targets is restricted by the lipophilic membrane of the cell. Antibodies that do function within the cell have been reported (Kontermann, 2004; incorporated herein by reference). Such antibodies are translated within the target cell and are termed intrabodies. However, delivery of these intrabodies is problematic since the delivery method of DNA transfection is not desirable in patients (Gupta et al, 2005; incorporated herein by reference). Other molecules which may bind to intracellular targets (e.g. antibodies that bind cytosolic proteins in cell staining or Western blotting) rely on the cell membrane having first been compromised in some manner, meaning that they cannot be used in therapeutic methods since they cannot cross the cell membrane.

The current focus on delivering antibodies and other such binding moieties into a cell is via fusion with cell-penetrating peptides. Such peptides are generally derived from viruses which use a series of positively charged amino acids to gain access to mammalian cells (Dietz and Bahr, 2004; incorporated herein by reference). Another method involves the conjugation of the binding moiety with a supercharged green fluorescent protein (GFP). GFP in its ‘wild-type’ form has a net charge of −7, meaning that it has seven more negatively-charged amino acids than it has positively-charged amino acids (Lawrence et al ., J. AM. CHEM. SOC. 2007, 129, 10110-10112; incorporated herein by reference).

Modifications to GFP to confer a net charge of +36 (+1.27/KDa) have been proposed (Lawrence et al 2007; incorporated herein by reference). This +36 GFP variant was found to have the ability to internalise in mammalian cells. It has also been found to be able to internalise other proteins by fusion thereto. Such proteins are thought to retain their activity within the cell (Cronican et al 2010; incorporated herein by reference).

However, conjugation of highly charged GFP or cell-penetrating peptide to a binding moiety may not always be appropriate, or efficient for delivery. There is accordingly a need in the art for binding moieties that can be delivered intracellularly without being conjugated to another molecule. There is a need for binding molecules that will bind an intracellular target without the cell membrane having been compromised, i.e. molecules that are suitable for in vivo therapeutic use. There is a need for intracellular delivery of binding moieties that will specifically bind an intracellular antigen. There is a need for methods of making such binding moieties.

### SUMMARY

The present invention meets one or more of the above needs by providing a Designed Ankyrin Repeat Protein (DARPin) that specifically binds to an antigen, the DARPin comprising an N-terminal, at least two Ankyrin Repeat sections, and a C-terminal, characterised in that the DARPins have a net charge that is less negative (i.e. more positive) than the DAPRin of SEQ ID NO: 1, excluding the charge contribution of the variable antigen-binding residues. In certain embodiments, the DARPins of the invention have a net charge of zero, excluding the charge contribution of the variable antigen-binding residues. In still other embodiments, the DARPins of the invention have a positive net charge, excluding the charge contribution of the variable antigen-binding residues. Accordingly, DARPins of the invention have a net charge that is greater than −14 (i.e., more positive), excluding the charge contribution of the variable antigen-binding residues. In specific embodiments, DARPins of the invention have a net charge of −13, −12, −11, −10, −9, −8, −7, −6, −5, −4, −3, −2, −1, or 0, excluding the charge contribution of the variable antigen-binding residues. In other specific embodiments, DARPins of the invention have a net charge of +1, +2, +3, +4, +5, +6, +7, +8, +9, +10, +11, +12, +13, +14, +16, or more, excluding the charge contribution of the variable antigen-binding residues.

In one aspect, the present invention provides a Designed Ankyrin Repeat Protein (DARPin) that specifically binds to an antigen, the DARPin comprising an N-terminal cap section, at least two Ankyrin Repeat (AR) module sections, and a C-terminal cap section, characterised in that the DARPin has a charge that is less negative than the DAPRin of SEQ ID NO: 1, excluding the charge contribution of the variable antigen-binding residues.

In another aspect, the present invention provides a method of making a Designed Ankyrin Repeat Protein (DARPin) capable of (i) binding an antigen; and (ii) crossing the membrane of a cell, the method comprising: a) generating a library of DARPins; b) carrying out a first selection using the antigen; c) carrying out a second selection using a negatively charged reagent; d) eluting the DARPins; and e) purifying the DARPins.

In yet another aspect, the present invention provides a DARPin library comprising a plurality of DARPins each comprising a DARPin framework sequence having an amino acid sequence according to SEQ ID NO: 8, wherein each member of the library has a charge that is less negative than the DAPRin of SEQ ID NO: 1, excluding the charge contribution of the variable antigen-binding residues.

In an additional aspect, the present invention provides a method of identifying a Designed Ankyrin Repeat Protein (DARPin) capable of (i) binding an antigen; and (ii) crossing the membrane of a cell, the method comprising: a) screening the library of any one of claims 34 to 42 for binding to the antigen by carrying out a selection using the antigen; and b) purifying the DARPins.

A DARPin of the invention may specifically bind to an intracellular antigen. A DARPin of the invention may be conjugated to another moiety, which may specifically bind to an intracellular antigen. Thus, a DARPin of the invention may be used as a carrier molecule to transport other moieties (antibodies, therapeutics) into a cell.

### BRIEF DESCRIPTION OF THE DRAWINGS

The present invention will now be described with reference to the following Figures, in which are shown:

FIG. 1: Sequence of DARPin modules. The DARPin consists of one N-Cap, three AR sections and one C-Cap. Each section has two alpha helices (a) and the AR and C-Cap sections also have a beta turn (βt). The “*” represents any of the 20 amino acids except cysteine, glycine or proline and the “‡” represents asparagine, histidine, or tyrosine.

FIG. 2: The phage display process. A DNA library (in this case of different DARPins) is introduced to phages. The phages then display the antibodies on the surface and those antibodies able to bind to an antigen will remain in the process while other antibodies are washed away. The retained antibodies can then be eluted and infect TG1 cells. The antibody sequences can then be analyzed from the cells and phages can be rescued for another round of selection. Illustration from Hoogenboom et al. (1998).

FIG. 3: Phage ELISA. Absorbance at 450 nm for eight different variants and an irrelevant DARPin (INSA1) on L-Myc, C-Myc, N- Myc, Mad, Max, CEA6 and no coating.

FIG. 4:—The original DARPin sequence with possible mutations from “wobbling” oligos located under the amino acids. The DARPin consists of one N-Cap, three AR sections and one C-Cap. Each section has two alpha helices (a) and the AR and C-Cap sections also have a beta turn (βt). The “*” represents any of the 20 amino acids except cysteine, glycine or proline. “‡” represents asparagine, histidine or tyrosine. Note that in the L-Myc library the first mutation in the AR's is only present in AR1 (L48R).

FIG. 5:—The charge selection strategy. A double selection on L-Myc and heparin was followed by a selection on heparin. Net average charge in output shown

FIG. 6: Average net charge from sequenced clones after each step in the selection strategy.

FIG. 7—Frequency (%) of sequenced DARPins with each net charge. From before heparin selections and after two rounds of heparin selection.

FIG. 8—Phage ELISA on L-Myc of 18 supercharged clones compared to template.

Absorbance at 450 nm as percentage of absorbance at 450 nm for template.

FIG. 9: Residue charge statistics after L-Myc selection and two selections on heparin (n=84) for all 22 mutated residues. Negative, neutral or positive residues as percent of total.

FIG. 10: Residue charge change from total library (n=18) to after L-Myc selection and phage rescue (n=39). Percentage point increase in positively charged residues at each mutated position.

FIG. 11: Residue charge change from after L-Myc selection (n=39) to after two rounds of heparin selection (n=84). Percentage point increase in positively charged residues at each mutated position.

FIG. 12A-B: Surface charge representation of the S-13 WT DARPin (left) S+11 scDARPin (right) at 4 different angles, Panel A: above and below, Panel B: side 1 and side 2. The shading represents the electrostatic surface potential from −1 kT/e to +1 kT/e and white being neutral. Regions showing an increase in positive charge are bounded with a dashed line. Modeling done in Accelrys Discovery Studio.

FIG. 13A-B: Transfer of positive charge to DARPins of other specificities; Effects on antigen binding: ELISA binding for seven different DARPins at a range of charge from negative (WT DARPin; ↓) to positive (↑), using permissive positions determined for prototype DARPin against L-Myc (S-11). Positive charges could be transferred to the DARPins without abolishing binding or specificity. Panel A: anti-insulin DARPin 1 (upper left); anti-insulin DARPin 2 (upper right); anti-insulin DARPin 3 (lower left); and anti-KRas DARPin (lower right). Panel B: anti-CD73 DARPin (upper left); anti-c-Myc DARPin upper right; and anti-L-Myc DARPin (lower middle).

FIG. 14A-B: Positively charged residues were introduced at different permissive sites in the cMYC DARPin framework region. 11 resultant charged mutant DARPin sequences (‘mut1’, ‘mut2’ etc) are shown. The parental DARPin is labelled as ‘WT’ and is the most negatively charged sequence in the set. All sequences were displayed on phage particles and an ELISA was performed to measure binding to the respective antigens or an irrelevant control protein. The ELISA signal (absorbance at 450 nm) is shown in the column ‘ELISA’ and in the next column ‘% WT SIGNAL’ is converted to a percentage of the parental signal in the ELISA. Positive amino acid side chains are marked in boxes. Variable positions which confer antigen binding are indicated by asterisks (*) above the sequences. Panel A shows the sequences of the N-terminal cap and the AR modules 1 and 2. Panel B shows the sequences of the AR module 3 and the C-terminal cap.

FIG. 15A-B: Positively charged residues were introduced at different permissive sites in the CD73 DARPin framework region. 11 resultant charged mutant DARPin sequences (‘mut1’, ‘mut2’ etc) are shown. The parental DARPin is labelled as ‘WT’ and is the most negatively charged sequence in the set. All sequences were displayed on phage particles and an ELISA was performed to measure binding to the respective antigens or an irrelevant control protein. The ELISA signal (absorbance at 450 nm) is shown in the column ‘ELISA’ and in the next column ‘% WT SIGNAL’ is converted to a percentage of the parental signal in the ELISA. Positive amino acid side chains are marked in boxes. Variable positions which confer antigen binding are indicated by asterisks (*) above the sequences. Panel A shows the sequences of the N-terminal cap and the AR modules 1 and 2. Panel B shows the sequences of the AR module 3 and the C-terminal cap.

FIG. 16A-B: Positively charged residues were introduced at different permissive sites in the LMYC DARPin framework region. 11 resultant charged mutant DARPin sequences (‘mut1’, ‘mut2’ etc) are shown. The parental DARPin is labelled as ‘WT’ and is the most negatively charged sequence in the set. All sequences were displayed on phage particles and an ELISA was performed to measure binding to the respective antigens or an irrelevant control protein. The ELISA signal (absorbance at 450 nm) is shown in the column ‘ELISA’ and in the next column ‘% WT SIGNAL’ is converted to a percentage of the parental signal in the ELISA. Positive amino acid side chains are marked in boxes. Variable positions which confer antigen binding are indicated by asterisks (*) above the sequences. Panel A shows the sequences of the N-terminal cap and the AR modules 1 and 2. Panel B shows the sequences of the AR module 3 and the C-terminal cap.

FIG. 17A-B: Positively charged residues were introduced at different permissive sites in the INS1 DARPin framework region. 11 resultant charged mutant DARPin sequences (‘mut1’, ‘mut2’ etc) are shown. The parental DARPin is labelled as ‘WT’ and is the most negatively charged sequence in the set. All sequences were displayed on phage particles and an ELISA was performed to measure binding to the respective antigens or an irrelevant control protein. The ELISA signal (absorbance at 450 nm) is shown in the column ‘ELISA’ and in the next column ‘% WT SIGNAL’ is converted to a percentage of the parental signal in the ELISA. Positive amino acid side chains are marked in boxes. Variable positions which confer antigen binding are indicated by asterisks (*) above the sequences. Panel A shows the sequences of the N-terminal cap and the AR modules 1 and 2. Panel B shows the sequences of the AR module 3 and the C-terminal cap.

FIG. 18A-B: Positively charged residues were introduced at different permissive sites in the INS2 DARPin framework region. 11 resultant charged mutant DARPin sequences (‘mut1’, ‘mut2’ etc) are shown. The parental DARPin is labelled as ‘WT’ and is the most negatively charged sequence in the set. All sequences were displayed on phage particles and an ELISA was performed to measure binding to the respective antigens or an irrelevant control protein. The ELISA signal (absorbance at 450 nm) is shown in the column ‘ELISA’ and in the next column ‘% WT SIGNAL’ is converted to a percentage of the parental signal in the ELISA. Positive amino acid side chains are marked in boxes. Variable positions which confer antigen binding are indicated by asterisks (*) above the sequences. Panel A shows the sequences of the N-terminal cap and the AR modules 1 and 2. Panel B shows the sequences of the AR module 3 and the C-terminal cap.

FIG. 19A-B: Positively charged residues were introduced at different permissive sites in the INS3 DARPin framework region. Nine resultant charged mutant DARPin sequences (‘mut1’, ‘mut2’ etc) are shown. The parental DARPin is labelled as ‘WT’ and is the most negatively charged sequence in the set. All sequences were displayed on phage particles and an ELISA was performed to measure binding to the respective antigens or an irrelevant control protein. The ELISA signal (absorbance at 450 nm) is shown in the column ‘ELISA’ and in the next column ‘% WT SIGNAL’ is converted to a percentage of the parental signal in the ELISA. Positive amino acid side chains are marked in boxes. Variable positions which confer antigen binding are indicated by asterisks (*) above the sequences. Panel A shows the sequences of the N-terminal cap and the AR modules 1 and 2. Panel B shows the sequences of the AR module 3 and the C-terminal cap.

FIG. 20A-B: Positively charged residues were introduced at different permissive sites in the KRAS DARPin framework region. 11 resultant charged mutant DARPin sequences (‘mut1’, ‘mut2’ etc) are shown. The parental DARPin is labelled as ‘WT’ and is the most negatively charged sequence in the set. All sequences were displayed on phage particles and an ELISA was performed to measure binding to the respective antigens or an irrelevant control protein. The ELISA signal (absorbance at 450 nm) is shown in the column ‘ELISA’ and in the next column ‘% WT SIGNAL’ is converted to a percentage of the parental signal in the ELISA. Positive amino acid side chains are marked in boxes. Variable positions which confer antigen binding are indicated by asterisks (*) above the sequences. Panel A shows the sequences of the N-terminal cap and the AR modules 1 and 2. Panel B shows the sequences of the AR module 3 and the C-terminal cap.

FIG. 21A-C: Sequence alignment showing the 15 most positively charged DARPins (derived from INS1, INS3, KRAS, LMYC and CD73 ‘parental’ DARPins) which retained >50% antigen binding activity as determined by ELISA. Positive amino acid side chains are marked in boxes. Variable positions which confer antigen binding are indicated by asterisks (*) above or within the sequences. The ELISA signal (absorbance at 450 nm) is shown in the column ‘ELISA’ and in the next column ‘% WT SIGNAL’ is converted to a percentage of the parental signal in the ELISA. Below the alignment is a comparison of the original DARPin library design (net charge of −14, or −0.77/kDa) to a charged DARPin library design (net charge of +16, or 0.88/kDa). The charged library design incorporates positive charge at all the permissive sites. Panel A: the N-terminal cap sequences. Panel B: the AR module 1 and AR module 2 sequences. Panel C: the AR module 3 and C-terminal cap sequences.

FIG. 22: Internalization shown by flow cytometry histogram. HeLa cells are treated with 200 nM S+11 scDARPin or +36 GFP. n=1.

FIG. 23:—Internalization is exclusive to scDARPins by flow cytometry (Left panel). HeLa cells treated with 200 nM S+11 scDARPin, S−13 tDARPin, S+4 scDARPin or Alexa Fluor 488. n=1. Internalization of S+9 and S+11 scDARPin by image flow cytometry (Right panel). HeLa cells treated with 50 nM S+9 or S+11 scDARPin. n=3 (S+9) and n=2 (S+11).

FIG. 24: Energy dependent internalization by flow cytometry. HeLa cells are incubated at 4 C for 1 h with 100 nM S+11 scDARPin and washed with either PBS or PBS with heparin. As percentage of similar experiment at 37° C. n=1.

FIG. 25: Internalization score for +36 GFP (left) and s+11 scDARPin (right) by image flow cytometry. Positive values indicate internalization.

### DETAILED DESCRIPTION

Designed Ankyrin repeat proteins (DARPins) are binding molecules, which can be selected to bind a variety of targets. This antibody-like ability gives the protein some of the same functions as antibodies. DARPins have several properties that make them attractive as antibodies and as templates for introducing charged residues without losing their function.

DARPins are relatively small with a molecular weight of -17 kDa. This is about eight times lower than Immunoglobulin G antibodies. DARPins are very stable proteins and have melting temperatures (Tm) values ranging from 66 to above 85 (Binz et al., 2003). DARPins can have high affinity to their antigens with dissociation (K_{D}) values down to picomolar ranges (Zahnd et al., 2007). The study of DARPins is also greatly facilitated by their relatively easy production as they can be expressed in high amounts with up to 200 mg/l protein in simple shake flask culture in the low expressor strain XL1-blue (Binz et al., 2003). The DARPins are cysteine free, which prevents disulfide formation and allows them to be used as intracellular proteins (Binz et al., 2003). And additionally, DARPins are well suited to high throughput methods such as ribosome and phage display, which allows several billion variants to be tested against a given antigen.

DARPins are derived from natural ankyrin repeat proteins. The biological function of ankyrins is to mediate numerous key protein-protein interactions (Li et al., 2006). The ankyrins consists of repeat sections, which are stacked together to form a rigid protein domain (Li et al., 2006). These ankyrin repeat sections have been engineered in a consensus approach to form one ankyrin repeat (AR) module (Binz et al., 2003).

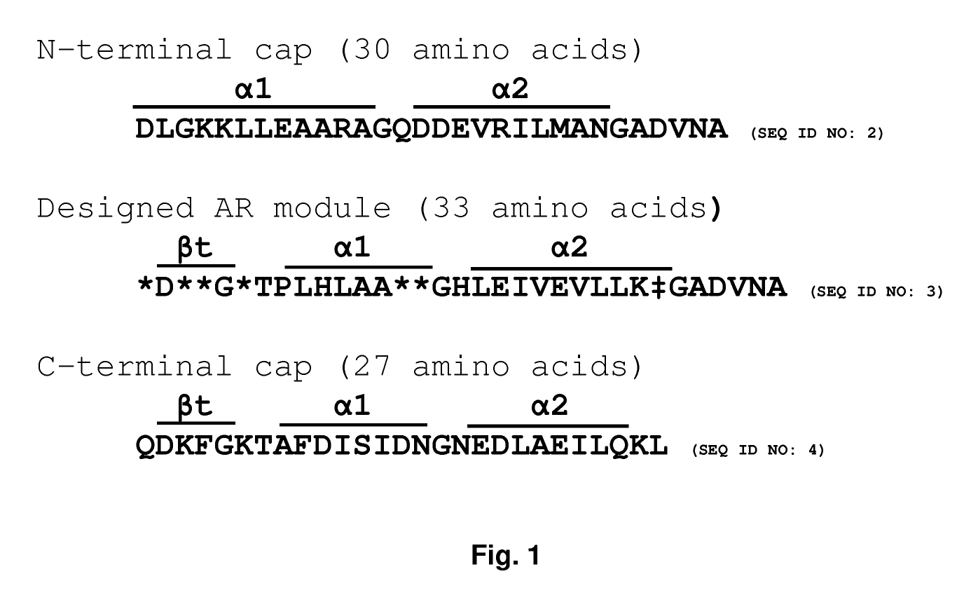

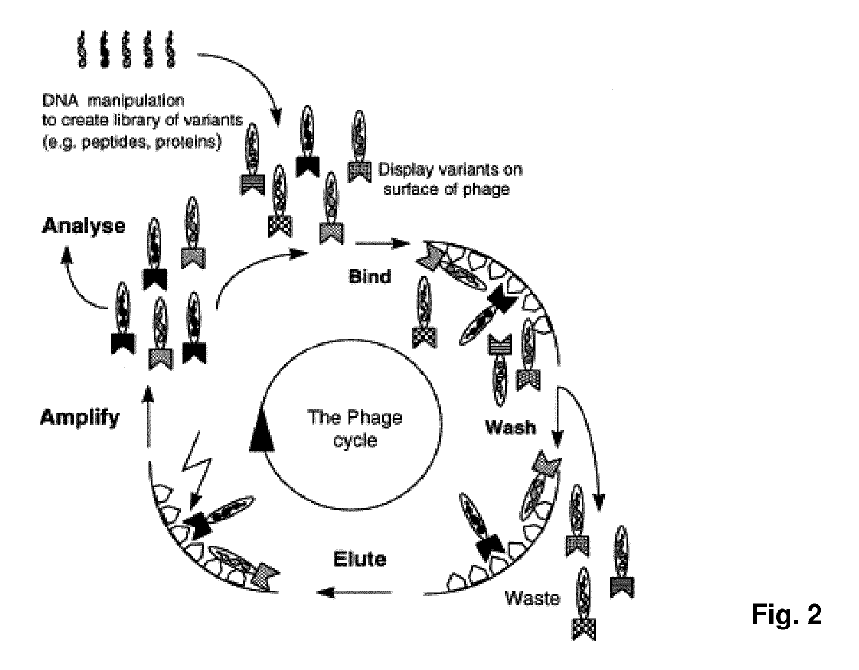

The repeat sections each contains seven variable positions and with three AR's the theoretical diversity reaches 10²³ (Binz et al., 2003), (FIG. 1). The repeats are flanked with an N- and C-Cap, that function to seal the hydrophobic core of the stack of AR's. They are essential for efficient folding in the cell and for avoiding aggregation (Interlandi et al., 2008). The N- and C-Cap have many charged residues (mostly negatively), which are important for the stability (Interlandi et al., 2008).

The DARPins are cysteine free, but a single cysteine can be introduced at the C-terminus (Simon 2011). This will allow the DARPins to be labeled with fluorescent probes, so the uptake into cells can be measured and visualized, without labeling lysines.

DARPins have previously been used against intracellular targets, such as the c-Jun N-terminal kinase (JNK) (Parizek et al., 2012). To insert this DARPin into the cell, the DNA of the DARPin was artificially transfected into the mammalian cell and then produced by the cell itself.

Also for intracellular application a DARPin against the epithelial cell adhesion molecule, EpCAM, which is effectively internalized by receptor mediated endocytosis, have been studied (Winkler et al., 2009). The DARPins were produced as dimers and fused to protamine, which bind siRNA complementary to the anti-apoptotic bcl-2 messenger RNA (mRNA), and proved to be internalized and facilitate tumor cell apoptosis.

One of the main challenges using this scaffold as a template is the high negative charge of the DARPins. The net charge depends on the number of charged residues in the variable positions, but the framework positions alone, (i.e., excluding the variable positions), contain a net charge of −14. The negatively charged residues are generally surface exposed, and mutations to positive residues will result in a double increase of the charge, which makes these residues ideal to mutate.

By changing several residues in the framework positions to lysines and arginines, the present inventors surprisingly have found that the negative net charge of DARPins can be reduced while maintaining stability and antigen binding.

More surprisingly the present inventors have found that DARPins (which normally have a high negative net charge) can be modified to have a positive net charge and still retain their function of binding to an antigen. Moreover, the inventors have found that DARPins having a high positive net charge (referred to herein as a “supercharged DARPin” (scDARPin) can internalise, i.e. it can cross the lipophilic membrane of a cell. This capacity for intracellular delivery is obviously advantageous. Thus, the present inventors have found that DARPins having a reduce negative charge and supercharged DARPins (scDARPins) capable of internalizing in a cell can be produced.

Reference to ‘charge’ or ‘net charge’ throughout means theoretical charge at neutral pH unless otherwise specified. In this case, theoretical net charge is expressed in terms of overall theoretical net charge (i.e. with reference to the absolute number of positively-charged versus negatively-charged amino acids), or in terms of theoretical charge per KiloDalton (KDa) (Cronican et al, 2011). The charge per KDa is simply the net charge divided by the molecular weight (in KDa) of the protein. Positively-charged amino acids are: Lysine (K) and Arginine (R); negatively-charged amino acids are Aspartic acid (D) and Glutamic acid (E). All other amino acids (including Histidine) are deemed to be neutral, or uncharged at physiological (neutral) pH. In such a calculation, K and L are assigned a +1 value while D and E are assigned a −1 value; all other amino acids are assigned a ‘0’ value. In view of the theoretical nature of the calculation, references throughout to “net charge” or “theoretical net charge” or “charge of about . . . ” should be understood as encompassing a range within 10% more or less of the value provided. Thus a reference to e.g. a net charge of about +11 should be understood as encompassing a net charge between +9.9 and +12.1; and a net charge of +0.6/KDa should be understood as encompassing a net charge between +0.54/KDa and +0.66/KDa.

Modifying the net charge can be done genetically by replacing a (positively- or negatively-) charged amino acid with an uncharged amino acid; by replacing an uncharged amino acid with a charged amino acid; and/or by replacing a charged amino acid with an oppositely-charged amino acid (i.e. replacing a negatively-charged amino acid with a positively-charged amino acid, or vice versa).

Charge modifications can also be made to proteins using chemical modifications. Protein cationization by chemical treatment with various diamines e.g., ethylenediamine, hexamethylenediamine or polyethylenimine (PEI) is able to alter the natural charge of a translated protein. In some cases this has been demonstrated to enable the chemically-treated proteins to enter mammalian cells via endocytosis (Kumugai, 1987; Futami, 2012) or pass through other natural barriers, such as the blood-brain barrier (Triguero, 1989). However, the limitation of such an approach is the high likelihood of chemically modifying amino acid side chains which are important for the protein's natural function (e.g. binding, catalysis etc) and therefore impairing the activity of the protein. Another limitation for commercial (e.g. pharmaceutical) use of the chemical approach would be the lack of control over batch-to-batch consistency. The chemical process will cationize at different locations within each protein molecule, resulting in the product of the reaction containing a range of proteins cationized at different positions, which may not be a reproducible process for manufacture.

Internalisation can readily be assessed by determining whether the DARPin affects cell function, either by studying whether it neutralises the intracellular antigen for which it is specific; or by conjugating e.g. a recombinase into a cell having an inactivated GFP gene: if the DARPin internalises, the GFP gene will be reactivated through removal of an intervening gene segment by the recombinase and the cell will exhibit fluorescence. A similar test can be carried out using luciferase.

The capability of a particular DARPin to internalise into a cell can be assessed in a number of other ways, such as flow cytometry, image flow cytometry or confocal microscopy, as set out in more detail below. Where internalisation is measured by flow cytometry, a DARPin of the present invention conjugated directly to a fluorophore may exhibit at least a 10-fold higher mean fluorescence index (MFI) by comparison with a control. A suitable control in this case could be a protein which has not been charge-modified for cell entry. A DARPin of the present invention may exhibit at least a 100-fold, or 200-fold increase in MFI by comparison with such a control. Such an increase is indicative that the DARPin of the invention is internalising into a cell. This method uses stringent cell surface washing with negatively charged reagents such as heparin to remove proteins associated with the external surface of cells in order to ensure that only internalised fluorescence is measured.

Previous attempts (Parizek et al, 2012) to use DARPins against intracellular targets, such as the c-Jun N-terminal kinase (JNK), have relied on transfecting the DNA of the DARPin into the cell; the cell then produces the DARPin. By contrast, the DARPins of the present invention are capable of internalising (crossing the cell membrane) and thus need not be transfected in such a manner; nor conjugated to another molecule.

The present inventors have moreover surprisingly found that a relatively low positive net charge can enable the DARPins of the present invention to be delivered intracellularly. Thus, by contrast with the +36 (+1.27/KDa) GFP described above, a DARPin of the present invention can function with a positive charge of +9 or around +0.5/KDa.

This is surprising because the minimum positive charge requirement for efficient internalisation of a molecule has been disclosed to be +0.75/KDa (Cronican et al, 2011).

Accordingly, the DARPins of the present invention may have a positive net charge of less than +0.75/KDa. This is advantageous because many intracellular target antigens are themselves positively charged and thus any binding moiety that has too high a positive charge may repel the antigen it is specific for. For example in a recent survey of all potential cancer drug targets (Patel et al Nat Rev Drug Discov. 2013 12:35), the most prevalent category (comprising 29% of all cancer targets) was that of transcription factors/transcription regulators. These are nuclear proteins whose primary role is to interact with DNA, often via positively charged patches on their own surfaces.

A DARPin of the invention may have net charge that is less negative (i.e. more positive) than the DAPRin of SEQ ID NO: 1, excluding the charge contribution of the variable antigen-binding residues.

A DARPin of the invention may have a net charge of zero, excluding the charge contribution of the variable antigen-binding residues.

A DARPin of the invention may have a positive net charge and methods of using the same to identify DARPins capable crossing the membrane of a cell.

A DARPin of the invention may have two AR repeats.

A DARPin of the invention may have three AR repeats.

A DARPin of the invention may have more than three AR repeats.

A DARPin of the invention may further comprise an N-terminal and/or C-terminal cap.

A DARPin of the invention may comprise an N-terminal cap having one or more substitutions at the amino acid residues D15, E17, 120, G25 and/or D27 numbered relative to SEQ ID NO: 2.

A DARPin of the invention may comprise at least one AR module having one or more substitutions at the amino acid residues L18, E19, E22 and/or D30 numbered relative to SEQ ID NO: 3.

A DARPin of the invention may comprise a C-terminal cap having one or more substitutions at the amino acid residues D14, E18, D19, E22, D24 and/or E27, numbered relative to SEQ ID NO: 4.

A DARPin of the invention may have one or more substitutions at the amino acid residues D15, E17, 120, G25, D27, L48, E49, E52, D60, N62, L81, E82, E85, D93, N95, L114, E115, E118, D126, N128, D143, E147, D148 and/or E151, numbered relative to SEQ ID NO: 1.

A DARPin of the invention may have one or more of the following substitutions: D15E/K/N, E17K, 120R, G25R, D27E/K/N, L48R, E49K, E52K, D60E/K/N, N62K, L81R, E82K, E85K, D93E/K/N, N95K, L114R, E115K, E118K, D126E/K/N, N128K, D143E/K/N, E147K, D148E/K/N and/or E151K, numbered relative to SEQ ID NO: 1.

A DARPin of the invention may have the mutations D15N, E17K, D27K, L48R, E49K, E52K, D60N, N62K, E85K, D93N, N95K, E118K, D126K, D143E, D148K, E151K, numbered relative to SEQ ID NO: 1. This DARPin has a net charge of about +11 (+0.6/KDa), excluding the charge contribution of the variable antigen-binding residues. A DARPin of the invention may additionally have one or more of the following mutations: E82K, E115K, N128K and E147K. A DARPin comprising each of the additional mutations has a net charge of about +18, excluding the charge contribution of the variable antigen-binding residues. It will be understood that the total net charge of a DARPin will also depend on the specific amino acid residues located at positions associated with antigen-binding, i.e. 31, 33, 34, 36, 44, 45, 57, 64, 66, 67, 69, 77, 78, 90, 97, 99, 100, 102, 110, 111, and 123, numbered relative to SEQ ID NO: 1, 8 or 9.

In one embodiments, a DARPin of the invention comprises the amino acid sequence of SEQ ID NO: 8. In one embodiment, a DARPin of the invention comprises SEQ ID NO: 8 wherein X₁, X₉, and X₁₄ are N; X₂, X₅, X₇, X₈, X₁₀, X₁₃, X₁₅, X₁₈, X₁₉, X₂₃, and X₂₄ are K; X₃ is I; X₄ is G; X₆ is R; X₁₁, X₁₆, X₂₅, and X₂₆ are L; and X₁₂, X₁₇, X₂₁, and X₂₂ are E.

In certain embodiments, the residues associated with antigen-binding, i.e. 31, 33, 34, 36, 44, 45, 57, 64, 66, 67, 69, 77, 78, 90, 97, 99, 100, 102, 110, 111, and 123, numbered relative to SEQ ID NO: 1, 8 or 9 are generally not substituted. Thus, even at a positive net charge, a DARPin of the invention can retain binding to its antigen. This is preferable where a preselected DARPin binding an antigen of interest is engineered to have a positive net charge.

The present invention also provides libraries of of DARPins having net charge that is less negative (i.e. more positive) than the DAPRin of SEQ ID NO: 1, excluding the charge contribution of the variable antigen-binding residues.

The present invention also provides libraries of of DARPins having a net charge of zero, excluding the charge contribution of the variable antigen-binding residues.

The present invention also provides libraries of of DARPins having a positive net charge, excluding the charge contribution of the variable antigen-binding residues, and methods of using the same to identify DARPins capable crossing the membrane of a cell.

A library of DARPins of the invention comprises a plurality of DARPins each comprising the amino acid sequence of SEQ ID NO: 8.

In certain embodiments, a library of DARPins of the invention comprises a plurality of DARPins each comprising SEQ ID NO: 8, wherein:
(a) X₁, X₅, X₉, X₁₄, X₁₉, X₂₁, and X₂₃ are independently selected from the list consisting of Aspartate, Glutamate, Lysine, and Arginine;
(b) X₂, X₇, X₈, X₁₂, X₁₃, X₁₇, X₁₈, X₂₂, and X₂₄ are independently selected from Glutamate and Lysine;
(c) X₃ is selected from Isoleucine and Arginine;
(d) X₄ is selected from Glycine and Arginine;
(e) that X₆, X₁₁, X₁₆, X₂₅ and X₂₆ are independently selected from Leucine and Arginine; and
(f) X₁₀, X₁₅, and X₂₀ are independently selected from Asparagine and Lysine.

In certain embodiments, a library of DARPins of the invention comprises a plurality of DARPins each comprising SEQ ID NO: 8 wherein X₁, X₉, and X₁₄ are N; X₂, X₅, X₇, X₈, X₁₀, X₁₃, X₁₅, X₁₈, X₁₉, X₂₃, and X₂₄ are K; X₃ is I; X₄ is G; X₆ is R; X₁₁, X₁₆, X₂₅, and X₂₆ are L; and X₁₂, X₁₇, X₂₁, and X₂₂ are E.

A library of DARPins of the invention may comprise substitutions at residues associated with antigen-binding, i.e. 31, 33, 34, 36, 44, 45, 57, 64, 66, 67, 69, 77, 78, 90, 97, 99, 100, 102, 110, 111, and 123, numbered relative to SEQ ID NO: 1, 8 or 9. Such a library will be particularly useful for the identification of DARPins binding an antigen which have a net charge that is less negative (i.e. more positive) than the DAPRin of SEQ ID NO: 1 including DARPins having a positive net charge.

Where a particular antigen-binding specificity is to be maintained, the residues associated with antigen-binding, i.e. 31, 33, 34, 36, 44, 45, 57, 64, 66, 67, 69, 77, 78, 90, 97, 99, 100, 102, 110, 111, and 123, numbered relative to SEQ ID NO: 1, 8 or 9 are generally not substituted within a library of DARPins. Thus, a library of DARPins comprising a net charge that is less negative (i.e. more positive) than the DAPRin of SEQ ID NO: 1, including DARPins having a positive net charge, can be generated that generally retain binding to a antigen. This is preferable where a library of DARPins is generated from a preselected DARPin binding an antigen of interest. Such libraries may be screened to identify particular DARPins which retain antigen-binding and which have a desired net charge (e.g., a positive net charge).

The present invention also provides a method of making DARPins capable of binding an intracellular antigen by crossing the membrane of a cell, the method comprising:
(i) generating a library of DARPins;
(ii) carrying out a first selection using an antigen;
(iii) carrying out a second selection using a negatively charged reagent;
(iv) eluting the DARPins
(v) purifying the DARPins

In the method of the present invention, the library of DARPins comprises a plurality of DARPins each comprising the amino acid sequence of SEQ ID NO: 8. In certain embodiments, the plurality of DARPins each SEQ ID NO: 8 wherein X₁, X₉, and X₁₄ are N; X₂, X₅, X₇, X₈, X₁₀, X₁₃, X₁₅, X₁₈, X₁₉, X₂₃, and X₂₄ are K; X₃ is I; X₄ is G; X₆ is R; X₁₁, X₁₆, X₂₅, and X₂₆ are L; and X₁₂, X₁₇, X₂₁, and X₂₂ are E.

In the method of the present invention, the purification of the DARPins (step (v)) may be carried out in the presence of a salt buffer.

In the method of the present invention, the purification of the DARPins (step (v)) may be carried out in the presence of NaCl or KCl.

In the method of the present invention, the purification of the DARPins (step (v)) may be carried out in the presence of NaCl.

Although NaCl or KCl are the most commonly used salts during protein purification, others such as CaCl₂ or MgCl₂ could also be considered as substitutes (Guide to Protein Purification, Edited by Murray P. Deutscher, Methods in Enzymology, Academic Press). Further salts may also be known to those skilled in the art of protein purification.

The library of DARPins may be made by any suitable means, including phage display, yeast display, ribosome display, etc.

The first selection may be carried out using the antigen alone, or it may also include a negatively charged reagent. This selection isolates those DARPins which are specific for the antigen of interest.

The negatively charged reagent used in the selection step (iii) may be Heparin. Other negatively charged molecules are also contemplated and within the scope of this invention. The reagent may be DNA, a negatively charged protein such as albumin, a negatively charged small molecule, a negatively charged membrane, or a negatively charged resin. The second selection using a negatively charged reagent drives selection of DARPins having a positive charge.

The present inventors have surprisingly found that the second selection using a negatively charged reagent not only increases the average net charge of the variants, but also provides a significant increase in the number of variants having a high net charge (up to +11 or +14 or beyond) and which retain binding to the antigen. Given that DARPins are known to have a high (around −14) negative charge, this represents an unexpected outcome.

The present inventors have found that when the positive net charge of the DARPins increases, purification and elution becomes problematic. The present inventors have found that the addition of a salt such as NaCl (sodium chloride) to the elution buffer solves the problem. The concentration required will vary depending on the positive net charge of the DARPin. Up to 1 M or even 1.5 M may be required for DARPins having a charge of up to +14 or +18. For DARPins having a charge of around +11, a lower concentration will be acceptable. For instance, concentrations of NaCl of less than 0.6 M or of around 0.1 M may be used.

The method of the invention may further comprise one or more optimisation steps to obtain a DARPin having a suitably affinity and potency to be suitable for in vivo therapeutic use.

Thus, a DARPin of the present invention is specific for its antigen and may bind with a K_{D} of 10⁻⁶ M or better (i.e. lower). A DARPin of the invention may bind with a K_{D} of 10 nanoMolar (nM) or less. A DARPin of the invention may bind with a K_{D} of 1 nanoMolar (nM) or less.

The DARPins of the present invention can bind to and neutralise the antigen for which they are specific.

The potency (EC₅₀) of the DARPins of the invention may be less than 1000 nanoMolar. The potency (EC₅₀) of the DARPins of the invention may be less than 100 nanoMolar. The potency (EC₅₀) may be in the range of 1-100 nM.

The binding characteristics of a DARPin for its antigen, including but not limited to specificity, equilibrium dissociation constant (K_{D}), dissociation and association rates (K_{off} and Kₒₙ respectively), can be measured using a variety of standard, known techniques such as equilibrium methods (e.g., enzyme-linked immunoabsorbent assay (ELISA) or radioimmunoassay (RIA)), or kinetics methods (e.g. surface plasmon resonance (BIACORE® or KINEXA®). In particular, methods commonly used for measuring antibody-antigen interaction may be readily applied to DARPins. In particular methods for measuring the disassociation constant “Kd” by a radiolabeled antigen binding assay (RIA) have been described (see. ,e.g., Chen, et al., (1999) J. Mol Biol 293:865-881). Methods and reagents suitable for determination of binding characteristics of a DARPin by BIACORE®, are known in the art and/or are commercially available (see, e.g., U.S. Pat. Nos. 6,294,391; 6,143,574). Moreover, equipment and software designed for such kinetic analyses are commercially available (e.g. BIACORE® A100, and BIACORE® 2000 instruments; Biacore International AB, Uppsala, Sweden). Similarly, methods for measuring the affinity of protein-protein interactions by KINEXA® have been described (Salimi-Moosavi et al. (2012) Anal. Biochem. 426:134-41).

### Key to SEQ ID NOs

SEQ ID NO: 1 A reference “Wild-Type” DARPin comprising three AR module sections. Variable antigen-binding residues are shown by * and ‡, where * can be any amino acid other than Glycine Proline or Cysteine and ‡ can be Asparagine, Histidine or Tyrosine.
SEQ ID NO: 2 “Wild-Type” N-terminal cap
SEQ ID NO: 3 “Wild-Type” AR module. Variable antigen-binding residues are shown by * and ‡, where * can be any amino acid other than Glycine, Proline or Cysteine and ‡ can be Asparagine, Histidine, or Tyrosine. A DARPin of the invention may comprise 2 or more AR modules, which may include a combination of Wild-Type and Supercharged modules.
SEQ ID NO: 4 “Wild-Type” C-terminal cap
SEQ ID NO: 5 “Supercharged” N-terminal cap. Residues which may be varied to introduce positive charges are shown as X₁₋₅. In certain aspects:
   X₁ and X₅ can independently be Aspartate, Glutamate, Lysine, or Arginine;
   X₂ can independently be Glutamate or Lysine;
   X₃ can independently be Isoleucine or Arginine; and
   X₄ can independently be Glycine or Arginine.
SEQ ID NO: 6 “Supercharged” AR module. Variable antigen-binding residues are shown by * and ‡, where * can be any amino acid other than Glycine, Proline or Cysteine and ‡ can be Asparagine, Histidine, or Tyrosine. Residues which may be varied to introduce positive charges are shown as X₇₋₁₀. In certain aspects:
   X₉ can be Aspartate, Glutamate, Lysine, or Arginine;
   X₇ and X₈ can independently be Glutamate or Lysine;
   X₆ can be Leucine or Arginine; and
   X₁₀ can be Asparagine or Lysine.
   A DARPin of the invention may comprise 2 or more AR modules, which may include a combination of Wild-Type and Supercharged modules.
SEQ ID NO: 7 “Supercharged” C-terminal cap. Residues which may be varied to introduce positive charges are shown as X₂₁₋₂₆. In certain aspects:
   X₂₁ and X₂₃ can independently be Aspartate, Glutamate, Lysine, or Arginine;
   X₂₂ and X₂₄ can independently be Glutamate or Lysine; and
   X₂₅ and X₂₆ can independently be Leucine or Arginine.
SEQ ID NO: 8 A reference “Supercharged” DARPin. Variable antigen-binding residues are shown by * and ‡, where * can be any amino acid other than Glycine, Proline or Cysteine and ‡ can be Asparagine, Histidine, or Tyrosine. Residues which may be varied to introduce positive charges are shown as X₁₋₂₆. In certain aspects:
   X₁, X₅, X₉, X₁₄, X₁₉, X₂₁, and X₂₃ can independently be Aspartate, Glutamate, Lysine, or Arginine;
   X₂, X₇, X₈, X₁₂, X₁₃, X₁₇, X₁₈, X₂₂, and X₂₄ can independently be Glutamate or Lysine;
   X₃ can be Isoleucine or Arginine;
   X₄ can be Glycine or Arginine;
   X₆, X₁₁, X₁₆, X₂₅ and X₂₆ can independently be Leucine or Arginine; and
   X₁₀, X₁₅, and X₂₀ can independently be Asparagine or Lysine
   In other aspects X₃, X₄, X₂₀ and X₂₂ are not substituted (SEQ ID NO: 9)
SEQ ID NO: 9 A particular “Supercharged” DARPin similar to SEQ ID NO: 8, wherein X₃, X₄, X₂₀ and X₂₂ are not substituted
SEQ ID NO: 10 A particular “Supercharged” DARPin similar to SEQ ID NO: 8, wherein X₃, X₄, X₁₁, X₁₂, X₂₀ and X₂₂ are not substituted; and X₂₁ is a Glutamate
SEQ ID NO: 11 A particular “Supercharged” DARPin. Variable antigen-binding residues are shown by * and ‡, where * can be any amino acid other than Glycine, Proline or Cysteine and ‡ can be Asparagine, Histidine, or Tyrosine. In certain aspects:
   X₁, can be Lysine or Arginine.
SEQ ID NO: 12 N-terminal cap from a DARPIN disclosed in WO 2012/069655. Sequence may optionally comprise a G or G S at the N-terminal; position 22 may optionally be V, I or A. Sequences highlighted in grey are residues which differ from the wild-type N-cap.
SEQ ID NO: 13 N-terminal cap from a DARPIN disclosed in WO 2012/069655. Sequence may optionally comprise a G or G S at the N-terminal; position 22 may optionally be V, I or A. Sequences highlighted in grey are residues which differ from the wild-type N-cap.
SEQ ID NO: 14 C-terminal cap from a DARPIN disclosed in WO 2012/069655. Sequences highlighted in grey are residues which differ from the wild-type C-cap.
SEQ ID NO: 15 WT LMYC binding DARPin. Residues shown in lower case letters are antigen binding residues.

```
(156 amino acids)
SEQ ID NO: 1
DLGKKLLEAARAGQDDEVRILMANGADVNA*D**G*TPLHLAA**GHLEIVEVLLK‡GADVNA*D
**G*TPLHLAA**GHLEIVEVLLK‡GADVNA*D**G*TPLHLAA**GHLEIVEVLLK‡GADVNAQ
DKFGKTAFDISIDNGNEDLAEILQKL
(156 amino acids) with numbering
SEQ ID NO: 1
D L G K K L L E A A R A G Q D D E V R I L M A N G A D V N A
            5 10 15 20 25 30
* D * * G * T P L H L A A * * G H L E I V E V L L K ‡ G A D
              35 40 45 50 55 60
V N A * D * * G * T P L H L A A * * G H L E I V E V L L K ‡
              65 70 75 80 85 90
G A D V N A * D * * G * T P L H L A A * * G H L E I V E V L
              95 100 105 110 115 120
L K ‡ G A D V N A Q D K F G K T A F D I S I D N G N E D L A
            125 130 135 140 145 150
E I L Q K L
            155
N-terminal cap1 (30 amino acids)
SEQ ID NO: 2
D L G K K L L E A A R A G Q D D E V R I L M A N G A D V N A
                5 10 15 20 25 30
Designed AR module (33 amino acids)
SEQ ID NO: 3
* D * * G * T P L H L A A * * G H L E I V E V L L K ‡ G A D V N A
                5 10 15 20 25 30
C-terminal cap (27 amino acids)
SEQ ID NO: 4
Q D K F G K T A F D I S I D N G N E D L A E I L Q K L
                5 10 15 20 25
“Supercharged” N-terminal cap (30 amino acids)
SEQ ID NO: 5
D L G K K L L E A A R A G Q X₁ D X₂ V R X₃ L M A N X₄ A X₅ V N A
                5 10 15 20 25 30
“Supercharged” AR module (33 amino acids)
SEQ ID NO: 6
* D * * G * T P L H L A A * * G H X₆ X₇ I V X₈ V L L K * G A X₉ V X₁₀ A
                5 10 15 20 25 30
“Supercharged” C-terminal cap (27 amino acids)
SEQ ID NO: 7
Q D K F G K T A F D I S I X₂₁ N G N X₂₂ X₂₃ L A X₂₄ I L Q K L
                5 10 15 20 25
(156 amino acids)
SEQ ID NO: 8
D L G K K L L E A A R A G Q X₁ D X₂ V R X₃ L M A N X₄ A X₅ V N A
                5 10 15 20 25 30
* D * * G * T P L H L A A * * G H X₆ X₇ I V X₈ V L L K ‡ G A X₉
              35 40 45 50 55 60
V X₁₀ A * D * * G * T P L H L A A * * G H X₁₁ X₁₂ I V X₁₃ V L L K ‡
                  65 70 75 80 85 90
G A X₁₄ V X₁₅ A * D * * G * T P L H L A A * * G H X₁₆ X₁₇ I V X₁₈ V L
                  95 100 105 110 115 120
L K ‡ G A X₁₉ V X₂₀ A Q D K F G K T A F D I S I X₂₁ N G N X₂₂ X₂₃ L A
            125 130 135 140 145 150
X₂₄ I L Q K L
              155
(156 amino acids)
SEQ ID NO: 9
D L G K K L L E A A R A G Q X₁ D X₂ V R I L M A N G A X₅ V N A
                5 10 15 20 25 30
* D * * G * T P L H L A A * * G H X₆ X₇ I V X₈ V L L K ‡ G A X₉
              35 40 45 50 55 60
V X₁₀ A * D * * G * T P L H L A A * * G H X₁₁ X₁₂ I V X₁₃ V L L K ‡
                65 70 75 80 85 90
G A X₁₄ V X₁₅ A * D * * G * T P L H L A A * * G H X₁₆ X₁₇ I V X₁₈ V L
                  95 100 105 110 115 120
L K ‡ G A X₁₉ V N A Q D K F G K T A F D I S I X₂₁ N G N E X₂₃ L A
            125 130 135 140 145 150
X₂₄ I L Q K L
              155
(156 amino acids)
SEQ ID NO: 10
D L G K K L L E A A R A G Q X₁ D X₂ V R I L M A N G A X₅ V N A
                5 10 15 20 25 30
* D * * G * T P L H L A A * * G H X₆ X₇ I V X₈ V L L K ‡ G A X₉
              35 40 45 50 55 60
V X₁₀ A * D * * G * T P L H L A A * * G H X₁₁ X₁₂ I V X₁₃ V L L K ‡
                  65 70 75 80 85 90
G A X₁₄ V X₁₅ A * D * * G * T P L H L A A * * G H X₁₆ X₁₇ I V X₁₈ V L
                  95 100 105 110 115 120
L K ‡ G A X₁₉ V N A Q D K F G K T A F D I S I E N G N E X₂₃ L A
            125 130 135 140 145 150
X₂₄ I L Q K L
              155
(156 amino acids)
SEQ ID NO: 11
D L G K K L L E A A R A G Q N D K V R I L M A N G A K V N A
                5 10 15 20 25 30
* D * * G * T P L H L A A * * G H R K I V K V L L K ‡ G A N
              35 40 45 50 55 60
V K A * D * * G * T P L H L A A * * G H L E I V K V L L K ‡
              65 70 75 80 85 90
G A X₁ V K A * D * * G * T P L H L A A * * G H L E I V K V L
              95 100 105 110 115 120
L K ‡ G A K V N A Q D K F G K T A F D I S I E N G N E K L A
            125 130 135 140 145 150
K I L Q K L
            155
SEQ ID NO: 12

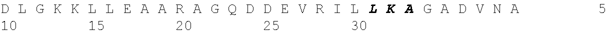

SEQ ID NO: 13

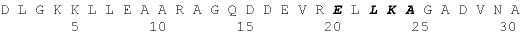

SEQ ID NO: 14

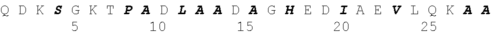

SEQ ID NO: 15
DLGKKLLEAARAGQDDEVRILMANGADVNAmDqyGfTPLHLAAwyGHLEIVEVLLKhGADVNAkDvhG
fTPLHLAAwtGHLEIVEVLLKnGADVNArDneGsTPLHLAAlaGHLEIVEVLLKnGADVNAQDKFGKT
AFDISIDNGNEDLAEILQKL
¹Domains defined as per WO 2012/069655 (this definition provides for three identical repeats)
```

### EXAMPLES

The present invention is illustrated by the following examples. It is to be understood that the particular examples, materials, amounts, and procedures are to be interpreted broadly in accordance with the scope and spirit of the invention as set forth herein.

### Example 1

### Production of Supercharged DARPins Against L-Myc and Other Antigens

The Myc proteins are transcription factors that regulate essential cellular processes. This includes cell growth, proliferation, cell cycle progression, transcription, differentiation, apoptosis and cell motility. Myc is part of a network of two other protein families. Max can bind to Myc and induce growth or it can bind to Mad and induce the opposite effect with growth arrest and differentiation (Hurlin and Dezfouli, 2004). This essential process is tightly controlled, and failure to control it can lead to tumorigenesis.

Myc is almost exclusively expressed in proliferating cells, while Mad is mostly expressed in non- proliferating cells (Luscher, 2001). This makes Myc an attractive target for cancer treatment, especially as Myc over-expression is found in most human cancers (Luscher, 2001).

Myc works by dimerization of its basic region/helix-loop-helix/leucine zipper (bHLHZip) domain with the same domain on Max (Luscher, 2001). Mad also has a bHLHZip domain that can dimerize with Max. A recombinant version of L-Myc's bHLHZip domain fused with a small ubiquitin-like modifier (SUMO) protein is used as the antigen in this study to generate a DARPin against L-Myc. This part of L-Myc contains two possible post translational modification sites. Putative serine and threonine phosphorylation sites have been detected by sequence similarity analysis.

### Generation of Supercharged L-Myc Binding DARPins

### Phage Display

The use of virus particles that can infect prokaryotic cells called bacteriophages or simply phages has since 1985 been used for high through put screening in phage display (Smith, 1985). The M13 phage particle, favored for phage display, consists of several coat proteins. Most noteworthy are protein 8 (p8), which coat the phage with several thousand copies and protein 3 (p3), that can bind the F pilus of bacteria and enable infection. Inside the phage is genomic single stranded DNA (ssDNA) (Marvin, 1998).

In the phage display system the genomic DNA has been replaced with a phagemid vector, that has antibiotic resistance and encodes a fusion protein of p3 and a protein of interest (Winter et al., 1994), in this case a DARPin antibody. The phagemid does not encode any viral structural or replication genes. Only after addition of helper phages, which contribute these missing genes, can a new phage particles form in the bacteria.

By creating mutations in the DARPin antibody (or any other protein of interest) in the phagemid vector, a library of DARPin antibodies can be created. This library can then be transformed into TG1 E. coli cells and after addition of helper phages a library of phages is created, where one phage particle contains the gene for one DARPin (Winter et al., 1994).

The phages are then used in selections against an antigen (Hoogenboom et al., 1998), in this case the L-Myc protein. The library of phages is added to a well coated with the antigen, and is followed by several washing steps to remove any DARPin-phages without affinity to the antigen (FIG. 2).

The retained DARPin-phages can then be eluted with trypsin. p3 encoded from the helper phages has a trypsin cleavage site inserted, which leave them uninfective after trypsin treatment. In contrast are the p3's encoded by the phagemid not trypsin cleavable, but a myc-tag between p3 and the DARPin is. This releases the phage and leaves an intact p3 protein, rendering these phages infective.

After infection of TG1 E. coli cells, the selected DARPins can be analyzed or rescued by helper phages to amplify the DARPins for a new round of selection. Several rounds of selection can then enrich the best binders, as these will have a competitive advantage during the selection.

A selected antibody can then further improve its affinity to its antigen by making random mutations in the binding regions and thereby creating a new library that again are selected against the antigen. In this study these secondary mutations are instead made in the non-binding areas where positively charged amino acids are inserted to increase the possibility of binding to negatively charged proteoglycans, and enhancing cellular uptake.

### Phage Display Panning

The first step to generate a supercharged DARPin was to isolate a DARPin with specificity for L-Myc. This was performed through phage display with a DARPin library of 10¹² phages, with a diversity of 10⁹. One well in a Nunc MaxiSorp plate was coated with an SUMO-L-Myc fusion protein at 1 μg/ml overnight (4° C.). The plate was rinsed 3 times with PBS to remove unbound antigen. The well was blocked with milk powder (Marvel, 3%) in PBS (MPBS) for 1 h at room temperature (RT). At the same time a 50 μL aliquot containing 10¹² phages was blocked with milk (6%) in 2×PBS (M2×PBS) for 1 hour at RT. The blocked phages were added to the blocked well and incubated at RT for 1 h. The well was washed 5 times with PBS+0.1% Tween-20 and 5 times with PBS.

The retained phages were eluted with trypsin (10 μg/mL in 0.1 M NaP buffer) for 0.5 hours at RT. The eluted phages were added to 900 μL of exponentially growing TG1 cells and incubated for 1 hour (37° C.). 900 μl of the cells were plated out on a 2×TYAG agar Bioassay plate and grown overnight (30° C.). The remaining 100 μ L of the cells were diluted and plated out on 2×TYAG agar plates to determine the output titers.

Phage Display Rescue

To rescue the phages, 5 ml of 2×TY was added to the Bioassay plates and the colonies were scraped off. The cell suspension was added to 25 ml of 2×TYAG to an OD600 of 0.1, and grown until the OD600 was between 0.5 and 1.0 (37° C., 280 rpm). M13KO7trp helper phages was then added and grown for 1 hour (37° C., 150 rpm). The culture was spun down (2000 g, 10 min.) and the supernatant discarded. The pellet was re-suspended in 25 ml 2×TYAK and grown overnight (25° C., 280 rpm). 1 ml of this culture was spun down in a micro centrifuge (maximum speed, 5 min.). 10 μL of the supernatant was deselected using SUMO-Mad fusion protein and at the same time blocked with M2×PBS (6%). The blocked phages can then be used as the input in the panning process.

Phage ELISA

After 3 rounds of selection, colonies were picked from the output titering and grown in 96 well Costar plates with 100 μl 2×TYAG overnight (37° C., 280 rpm). A glycerol stock was created by addition of 50 μl 50% glycerol and frozen at −80° C. From the glycerol stock 500 μl 2×TYAG media in a 2 ml 96 well plate was inoculated and grown for 5 hours (37° C., 280 rpm). 100 μl 2×TYAG media with 1.5*109 M13K07 helper phages were added and grown for 1 h (37° C., 150 rpm). The plate was spun down for 10 min (3200 rpm) and the supernatant discarded. 500 μl 2×TYAK media were added to the wells and grown overnight (25° C., 280 rpm).

The next day the cells were spun down (3200 rpm, 5 min) and 45 μl (per antigen well) supernatant were transferred to a Costar plate and 5 μl 30% M10×PBS were added to the wells and incubated for 1 hour (RT). At the same time a 96 well Nunc MaxiSorp plate coated with an SUMO-L-Myc fusion protein at 1 μg/mL (overnight, 4° C.) was washed 3 times with PBS and incubated for 1 hour with MPBS (3%, RT). The coated wells were washed 3 times with PBS and 50 μI of the blocked phages were transferred. The phages were incubated for 1 hour (RT) and then washed 3 times with 0.1% Tween-20 PBS. 50 l anti-M13-horse radish peroxidase (HRP) (1:5000 dilutions as recommended by the manufacturer, GE Healthcare) in MPBS (3%) were added to the wells and incubated for 1 h (RT). The wells were washed 3 times with 0.1% Tween-20 PBS and 50 μlitres Tetramethylbenzidine (TMB) substrate were added and developed for 5-20 minutes (RT). 50 μlitres 0.5 M H₂SO₄ were added to the wells and the fluorescence was measured at 450 nm.

### Design of Positively Charged DARPins

To aid in the creation of supercharged DARPins, structural data from the Protein Data bank (PDB, www.rscb.org) of other DARPins was used. Surface exposure of a previously crystallized DARPin (PDB: 3NOC) was calculated by Accelrys Discovery

Studio software package. Three crystal structures of DARPins bound to their antigen (PDB:2Y1L; PDB:3NOC; PDB:2J8S) were used to predict the proximity of the residues to the antigen. This was done in PyMol by choosing residues within 4.5 angstrom of the antigen. See Table 1 for additional information.

TABLE 1
Synthetic oligos used for supercharged library
creation.

| | |
|---|---|
| N-Cap: | CCGTATTGATCCATCGCGTTAACWTYTGCACSGTTCGCCATA |
| | AGTMTACGGACTTYATCWTYCTGCCCGGCACGCGCGGCTTCC |
| | AG SEQ ID NO: 16 |
| AR1 | GAAACCGTGGACATCTTTCGCWTTAACWTYTGCACCATGCTT |
| | CAACAGCACTTYCACAATTTYAMGGTGACCGTACCACG SEQ |
| | ID NO: 17 |
| AR2: | CGCTACCTCGTTATCGCGCGCWTTAACWTYTGCACCGTTCTT |
| | CAACAGCACTTYCACAATTTYGAGGTGACCCGTCCAC SEQ |
| | ID NO: 18 |
| AR3 | GGTTTTGCCAAACTTATCCTGAGCWTTCACWTYTGCACCGTT |
| | CTTCAACAGCACTTYCACAATTTYGAGGTGACCTGCCAG |
| | SEQ ID NO: 19 |
| C-Cap | GTGCGGCCGCCAGTTTCTGCAGGATTTYCGCTAAWTYTTYGT |
| | TGCCATTWTYAATGGAGATATCAAACGC SEQ ID NO: 20 |

Wobbling codons in bold. See description for explanation

### Library Creation

The library was created by oligo directed mutagenesis by the method of Kunkel (1985) on the template where 2 stop codons had been introduced. Briefly, a uracil containing template is prepared in E. coli CJ236 cells. The stop codons are then replaced when the mutagenic oligoes are annealed to the template. The original uracil containing template is destroyed when introduced to E. coli TG1 cells, and the new randomized sequences, can be rescued in phages.

### Supercharged Phage Display Panning

The panning process with the new supercharged library was performed like the first panning process on L-Myc, but performed as a double selection, on both L-Myc and heparin. Instead of eluting with trypsin the elution was performed with 100 μl 100 mM Triethylamine (TEA, pH 12.0). The TEA is added to the well and incubated for 5 min (RT). 50 μl TRIS-HCl (pH 8.0) is then added to neutralize the solution and the mixture is transferred to a new well coated with 150 μl 10 μg/ml heparin. The remaining panning and rescue procedure was the same as for the initial L-Myc selection.

For the second round of selection, the panning was again performed on 10 μg/ml heparin. The panning and rescue procedure was the same as for the initial L-Myc selection.

### L-Myc Selection

After three rounds of phage selection on L-Myc, phage ELISA was performed to assess the selected DARPins specificity for L-Myc. All the DARPins in the phage ELISA were sequenced and 8 were selected for having the highest L-Myc binding signal as well as having the most positively charged amino acid sequence. The selected variants ranged from having a net charge of −13 to −16, compared to the net charge of the non-variable positions of −14.

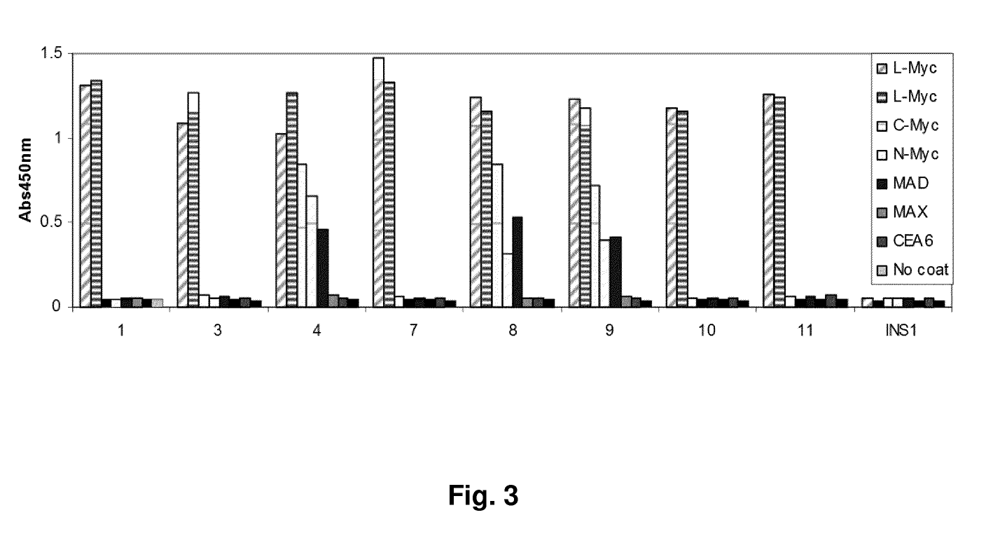

The 8 DARPins were then analyzed in a second phage ELISA, where their specificity for L-Myc versus the associated proteins C-Myc, N-Myc, Mad and Max, and an irrelevant protein CEA6 was determined (FIG. 3).

All of the DARPins still tested positive for L-Myc, but three of the clones also tested positive for C- Myc and N-Myc, however at a lower level. A DARPin with specificity for several of the Myc proteins could be used in more types of cancers, but these clones also had binding to the Mad protein, which has the opposite effect of Myc. Therefore DARPin 1, which only had specificity for L-Myc with a net charge of −13 was chosen as the template for the mutagenesis.

### Oligo Design for Library Creation

The template was mutated at 22-24 positions to lysine or arginine. A preference was given to polar, surface exposed, and negatively charged residues, which yields a double increase in net charge. Residues which have been reported as highly conserved in ankyrin repeat proteins (Mosavi et al., 2002, Binz et al., 2003), were avoided. Additionally residues close to the antigen were avoided, as predicted by their proximity in the amino acid sequence to the variable residues, and from crystal structures of other DARPins bound to their antigen. The same mutations were made in the 3 AR sections, although AR1 had one additional mutation.

The mutations were created in a “wobbling” approach, where oligos encoding either the original residue or a lysine/arginine residue were added to a mutagenesis reaction

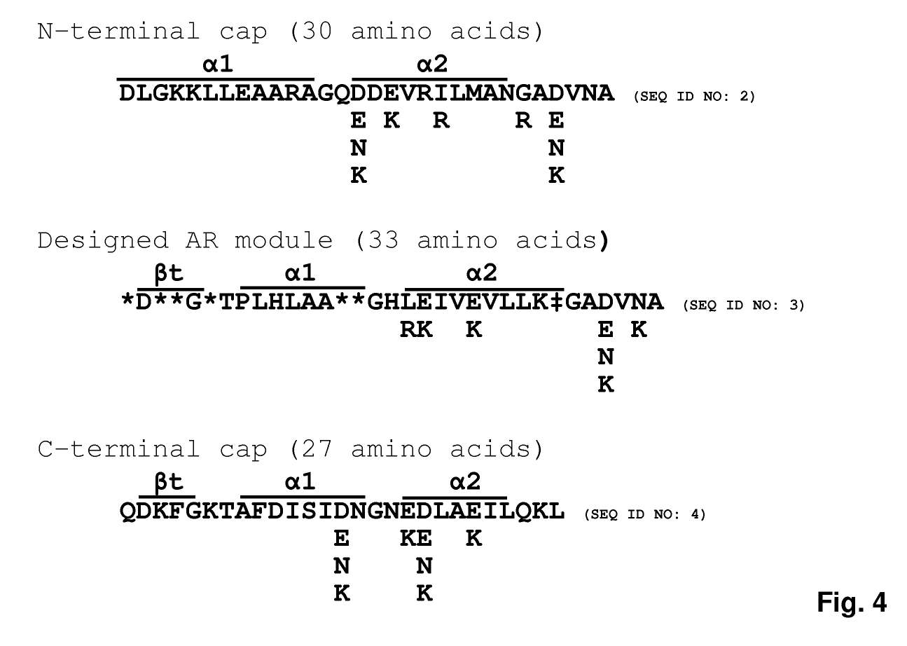

(FIG. 4). The oligos were synthetically created, where an equal mixture of 1 nucleotide that would yield a codon that translates to the original residue, and 1 nucleotide that would give a lysine/arginine, at the “wobbling” position. As it is not possible to change just 1 nucleotide to change aspartic acid to lysine or arginine 2 nucleotides were changes. This yields 4 possible codons that translate to aspartic acid, glutamic acid, aspargine or lysine. In total 5 oligos were created that each covered part of the 5 DARPin sections.

Using this approach a library with a diversity of 6×10⁸ and a net average charge of −1.7 was created. A single clone even had a net charge of +10, which represents a highly supercharged DARPin.

Charge Selection with Supercharged Library

The library was rescued by helper phages, to enable selection of the scDARPin proteins on L-Myc. The L-Myc concentration in the selections was raised from 1 to 10 μg/mL compared to the previous selections, to ensure that as few as possible DARPins with retained specificity for L-Myc would be lost at this stage. In fact, with an output of 10⁹ from an input of 10¹² this was not a concern.

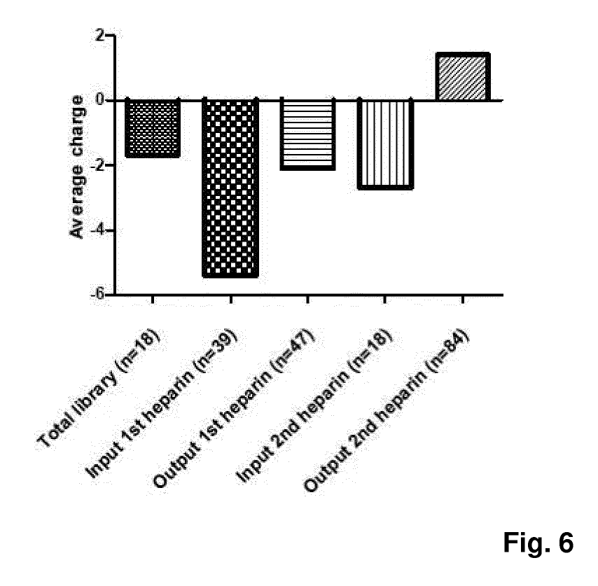

However the present inventors found at this stage the average net charge decreased from −1.7 to −5.4 (FIG. 6).

To overcome this problem, the inventors investigated selection using a negative reagent (in this case Heparin). Heparin is a relatively close mimic of heparan sulphate on the surface of mammalian cells (Chao et al., 2010). Heparin is a highly negative sulphated polysaccharide. The use of heparin as selection antigen is supported by the finding that Ribonuclease A's binding to heparin is much stronger than the binding of the closely related Onconase protein (Chao et al., 2010). And while the first is internalized by binding to proteoglycans, similarly to +36 GFP, the latter is bound and internalized in a similar, but proteoglycan independent manner (Chao et al., 2010).

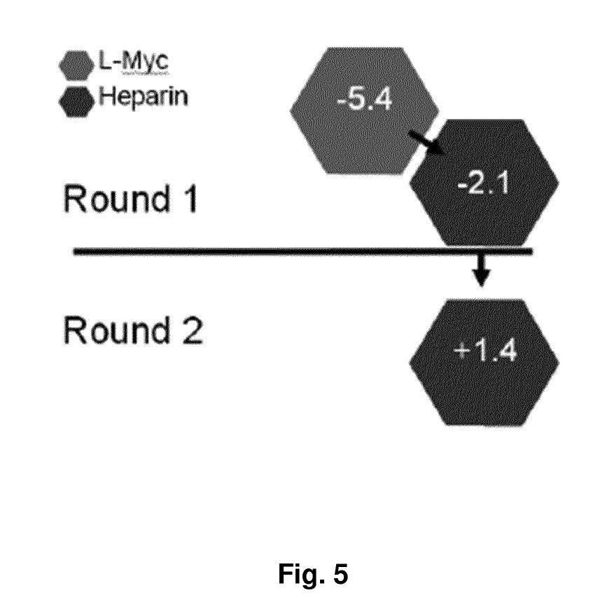

The selection on heparin was performed as a “double selection”, where the library was first selected on L-Myc and then eluted with TEA, to enable selection without a phage rescue on heparin (FIG. 5). This increased the net charge of the library from −5.4 to −2.1. After a phage rescue and a second selection on heparin the average net charge increased further to +1.4 (FIG. 6).

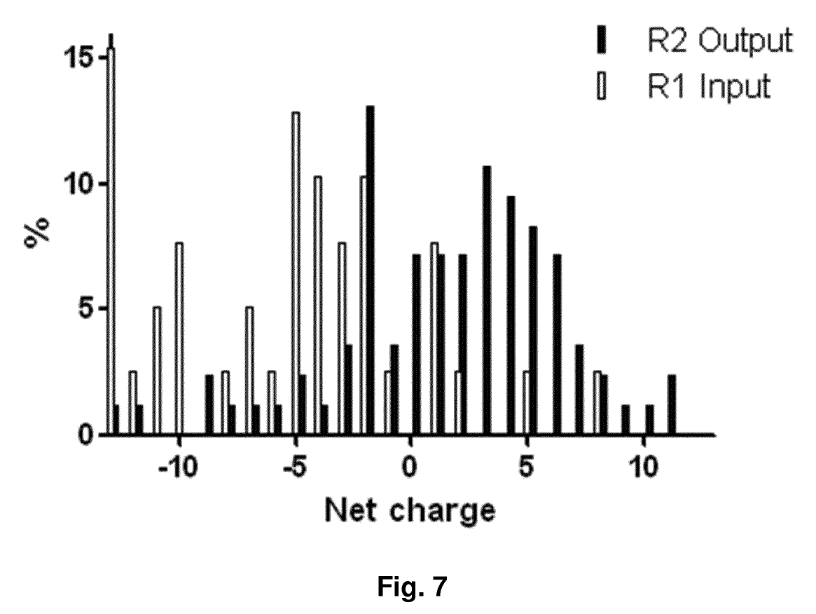

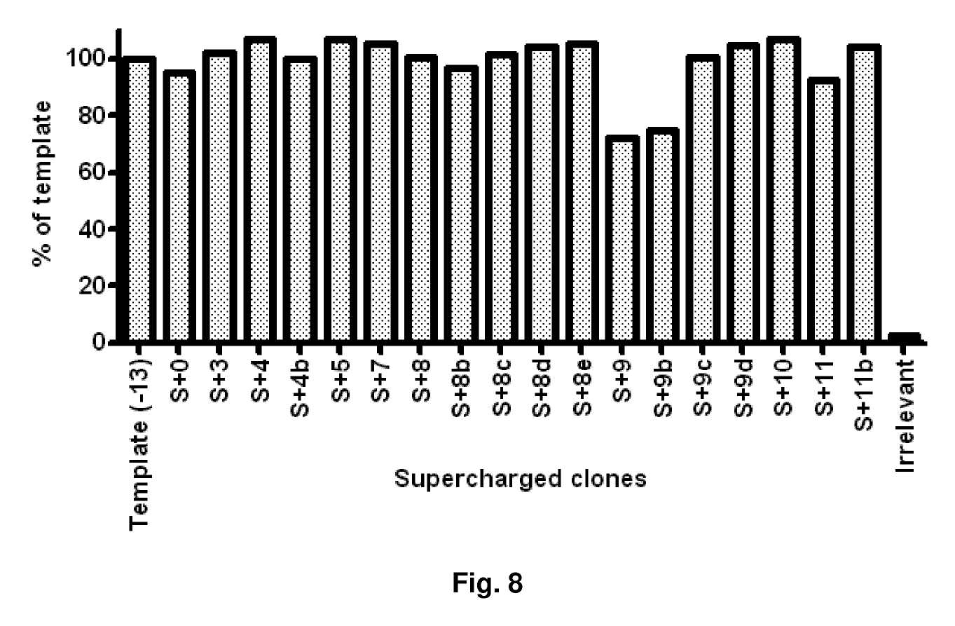

More significantly, the present inventors have surprisingly found that the number of variants with a high net charge increased very clearly (FIG. 7) as a result of this step.

From all the sequences, 18 DARPins (with either a high total net charge or a high net charge in the N- cap, all AR sections or the C-Cap) were chosen. The clone with the highest net charge had +11, an increase of 24 charges from the S-13 template DARPin (tDARPin) (Table 2). Note that all descriptions of charge are for the purified protein in a pQE-30 vector. When attached to the phage, the charge of the DARPins is one higher, due to an extra lysine residue at the C-terminus.

TABLE 2
Example of a scDARPin compared to the template. Overall net
charge, net charge in N-Cap, in all 3 AR sections and in C-
Cap. Table S-2 contains the full list of selected scDARPins

| | Overall | N-Cap | AR | C-Cap |
|---|---|---|---|---|
| Name | charge | charge | charge | charge |
| tDARPin S − 13 | −13 | −1 | −8 | −4 |
| scDARPin S + 11 | +11 | +4 | +7 | +0 |

Phage ELISA with Supercharged Clones

To determine if the supercharged clones retained the L-Myc specificity, phage ELISA was performed on L-Myc. 16 of the 18 clones retained 92-107% of the original binding signal to L-Myc, and 2 (S+9 and S+9b) clones retained about 70% of their original binding signal (FIG. 8).

### Variant Residues

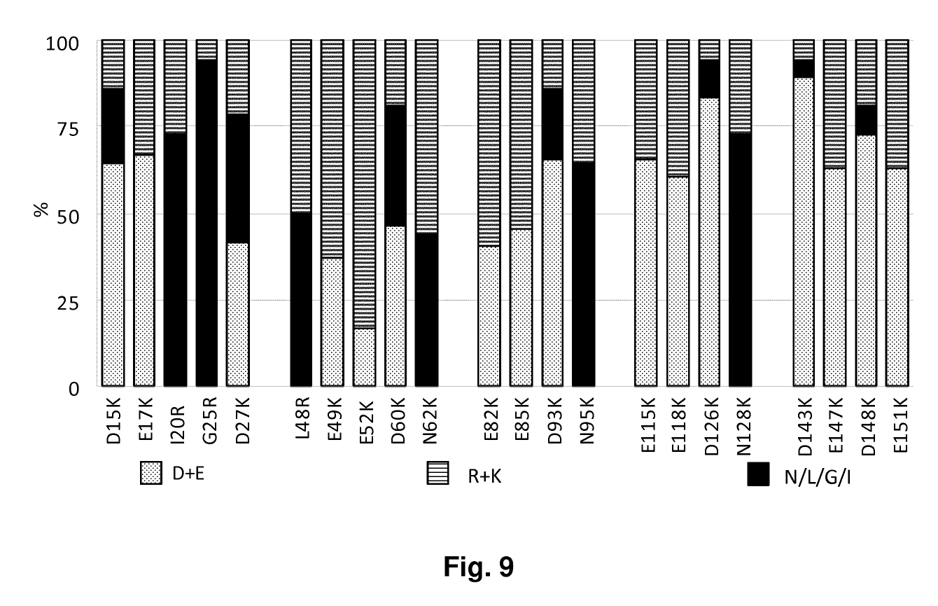

From the sequence data it was clear which residues were mutated most frequently. After selection on heparin, 6 of the residues were mutated to arginine or lysine with a frequency of at least 50% in the sequenced population (L48R, E49K, E52K, N62K, E82K, E85K) (FIG. 9). In addition, three residues were mutated in less than 10% of the clones (G25, D126, and D143).

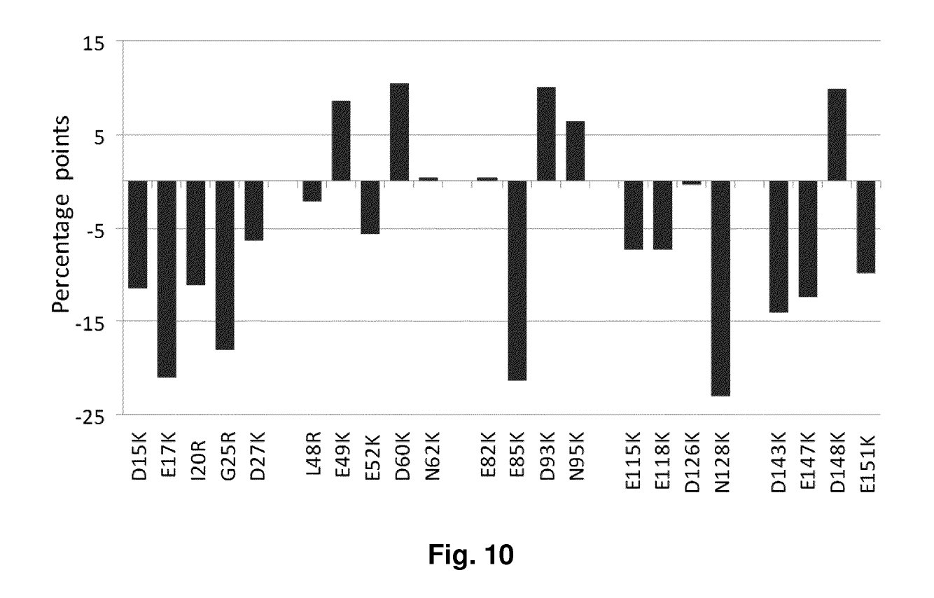

The sequence data also revealed which mutations were enriched the most during the selections. Comparing the initial library to the population after the L-Myc selection, five mutations were enriched 6-10 percentage points (FIG. 10).

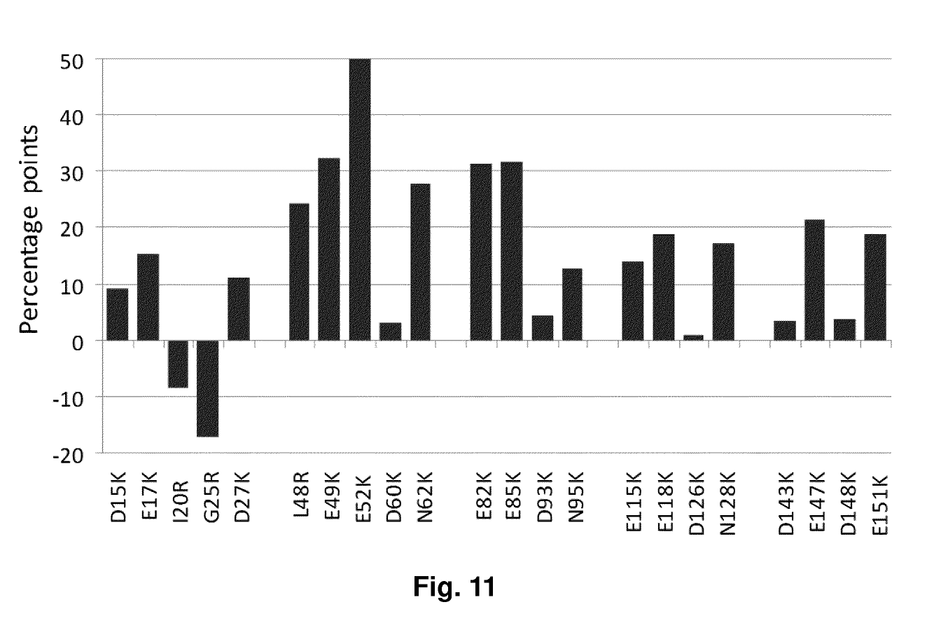

During the heparin selection, there is a general increase in positively charged residues, (FIG. 11). Without being bound by any theory, the present inventors suggest that those residues that are increased are likely to be exposed on the surface in a way that enhances heparin binding.

The present inventors suggest that the data presented herein are likely to be a good indicator for which residues to mutate in other DARPins (see below).

### Modelling

To increase the understanding of the created protein, a model of one L-Myc scDARPin was created. Like the surface exposure data, the model was built on the PDB file ‘3NOC’. This model was changed so the mutated and variable residues were like the sequence of the S-13 tDARPin or the S+11 scDARPin.

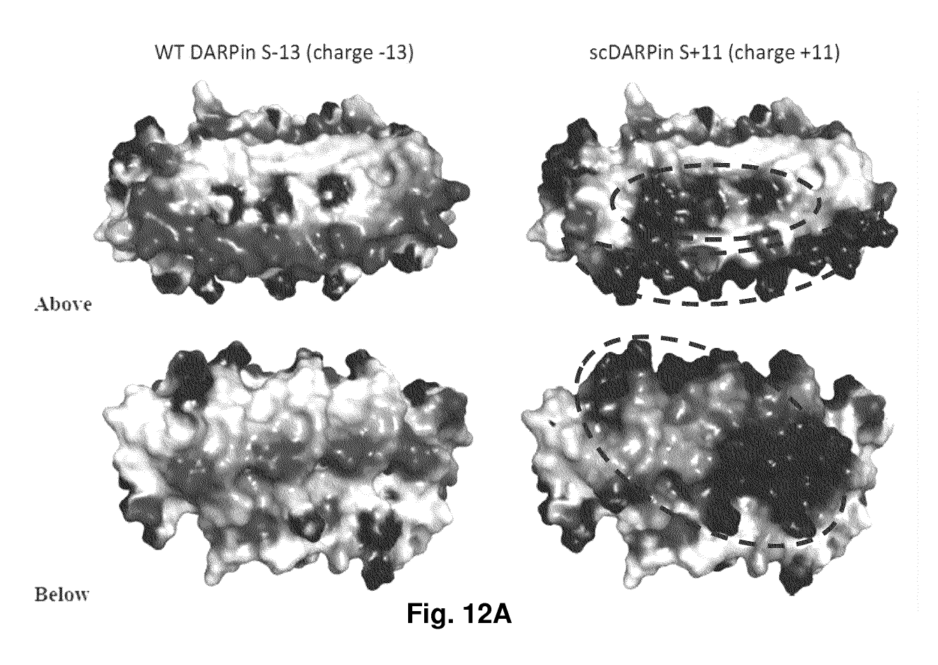

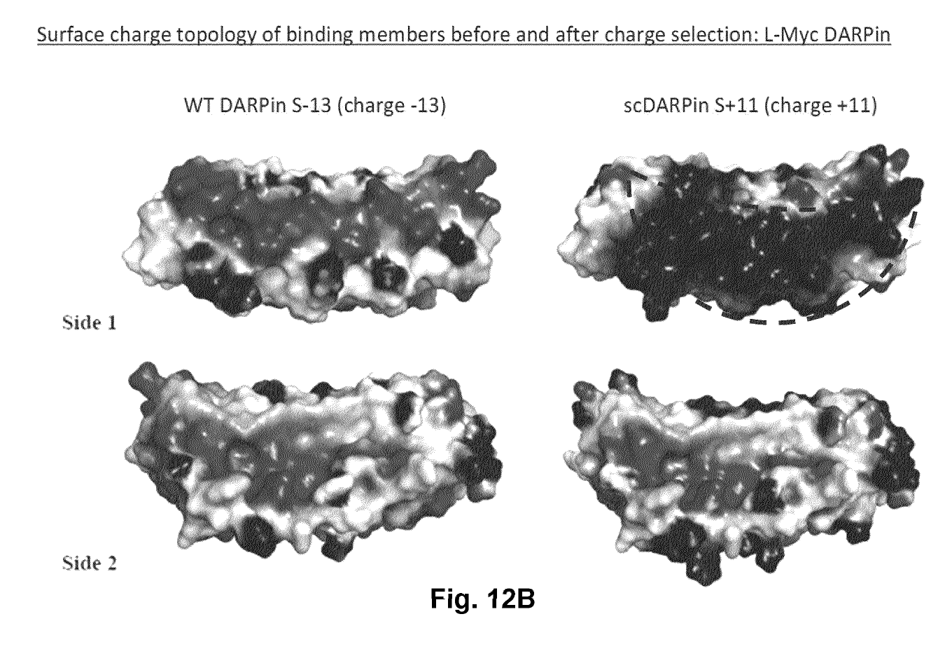

From these models the electrostatic surface potential was calculated and modeled (FIG. 12). From the surface charge representation it was revealed that a very positively charged area was present on what is described here as below and side 1 of S+11 scDARPin. It is most likely that this area bind to the heparin during the selection.

The negatively charged area on ‘side 2’ is the binding domain where the antigen is bound. The negative patch comes from five negative residues. Three come from the aspartic acid that starts each of the 3 AR regions and two are from the variable residues. Changing these residues is likely to result in a decrease or loss of antigen binding. Supercharging of another template DARPin with more positive residues in the variable domain would increase the net charge and possibly also the internalization.

This model may also be used when identifying which residues to mutate, when creating a more supercharged variant with point mutations.

Generation of Additional scDARPin Variants

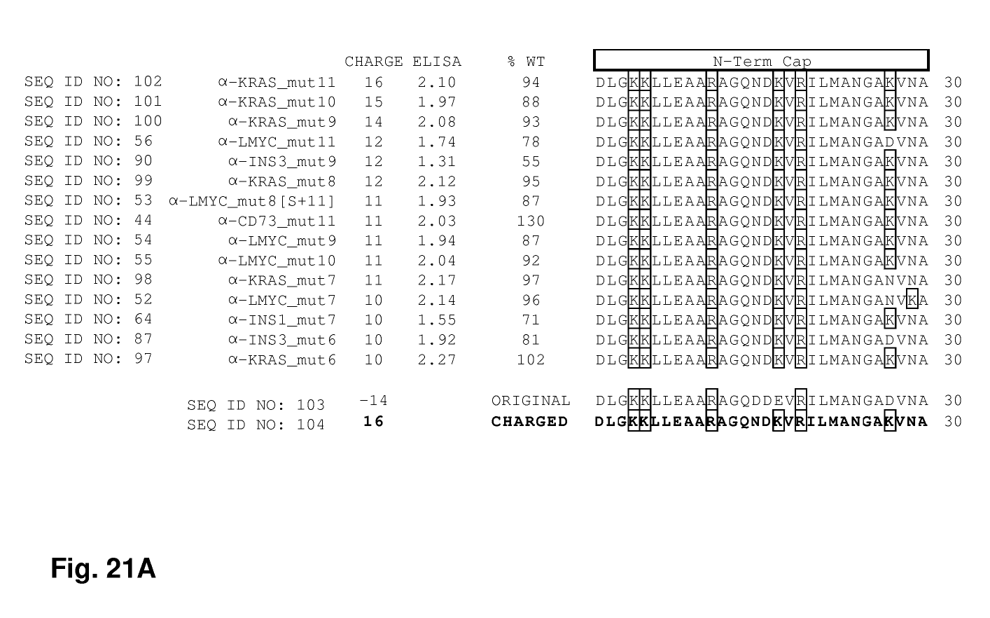

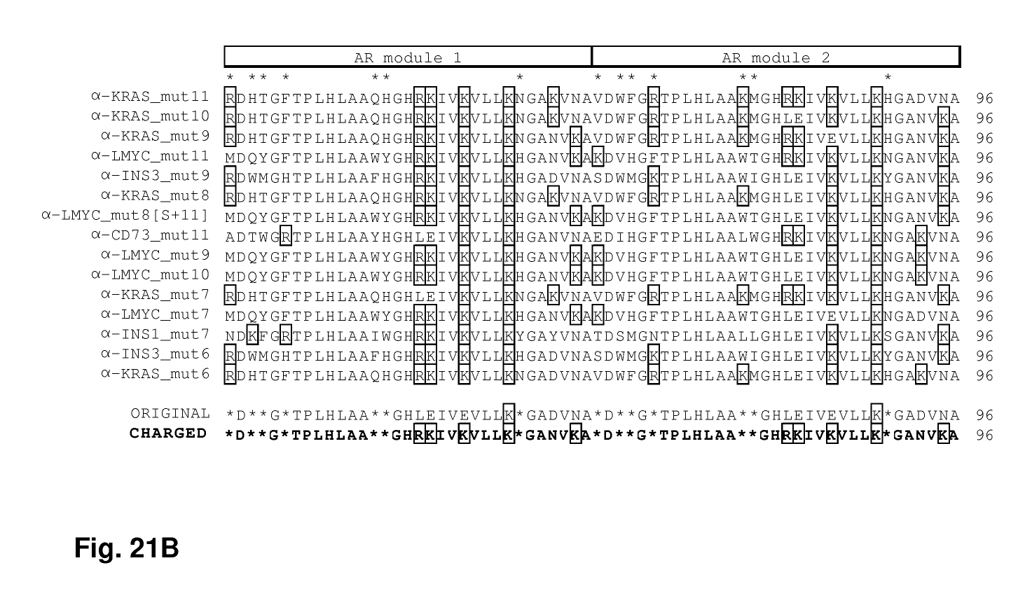

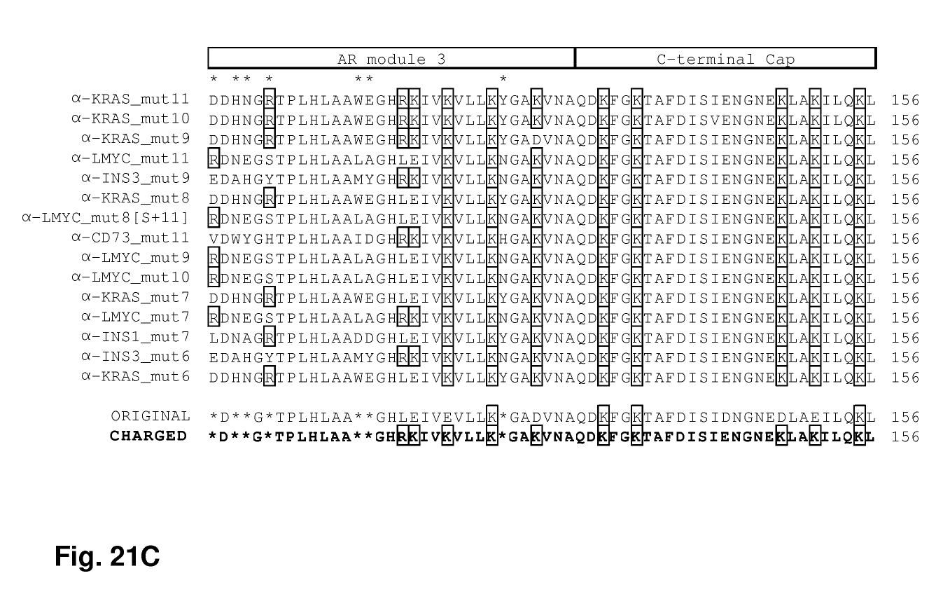

Using the knowledge of charge-permissive DARPin positions from the library selections on L-Myc, an attempt was made to transfer charges to other DARPins specific for different antigens. Six additional DARPins recognizing a variety of targets (i.e., C-Myc, CD73, INSULIN1, INSULIN2, INSULIN3, and KRAS) were isolated and characterized. A seventh DARPin, the original negatively charged L-Myc DARPin, was also included as a positive control. Using a mixture of six oligo's designed based on L-Myc S+11, up to 20 positions in each DARPin, depending on which combination of oligo's annealed, were mutated. The sequence and location of each primer is provided in Table 3. As noted in Table 3 several primers can anneal in multiple locations adding additional variability. Variants with a range of charges ranging from the wild-type (negatively charged) up to the most positively charged were picked for screening by ELISA on both the target antigen and an irrelevant antigen. For each of the six additional template DARPins numerous supercharged variants were identified having a net charge of at least +9 that maintained 50% or better binding activity (FIG. 21).

TABLE 3
Mutagenesis Primers (5′-3′)

| | | Changes Encoded‡ |
|---|---|---|
| | Mutaoenesis Oligo (5′ to 3′) | (bold, underlined) |
| 1 | GCGTTAACTTTTGCACCGTTCGCCATAA | RAGQNDKVRILMANGAK |
| | GTATACGGACTTTATCATTCTGCCCGGC | VN SEQ ID NO: 271 |
| | ACG (SEQ ID NO: 21) | |
| 2 | CAACAGCACTTTCACAATTTTACGGTGA | GHRKIVKVLL |
| | CC (SEQ ID NO. 22) | (SEQ ID NO. 28) |
| 3 | CGCTTTAACATTTGCACC (SEQ ID | GANVKA |
| | NO: 23) | (SEQ ID NO: 29) |
| 4 | CAACAGCACTTTCACAATTTCG (SEQ | EIVKVLL |
| | ID NO: 24) | (SEQ ID NO: 32) |
| 5 | CCTGAGCATTCACTTTTGCACC (SEQ | GAKVNAQ |
| | ID NO: 25) | (SEQ ID NO: 31) |
| 6 | CTGCAGGATTTTCGCTAATTTTTCGTTG | DISIENGNEKLAKILQ |
| | CCATTTTCAATGGAGATATC (SEQ ID | (SEQ ID NO: 32) |
| | NO: 26) | |

‡Primers 1 and 6 anneal to the N-term and C-term caps, respectively; Primers 3, 4 and 5 encode portions of the AR module and can anneal in multiple positions.

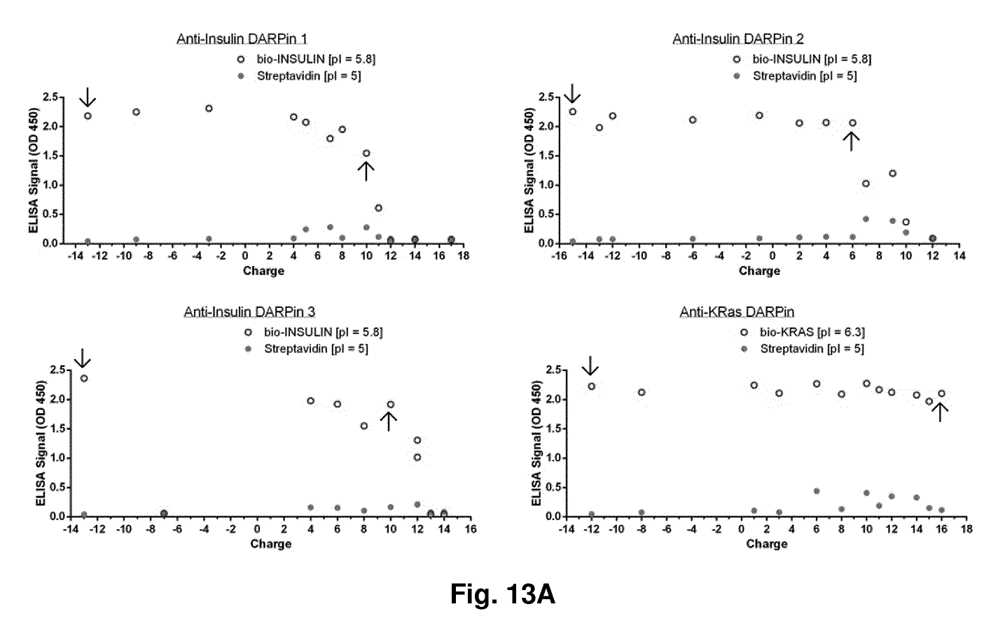

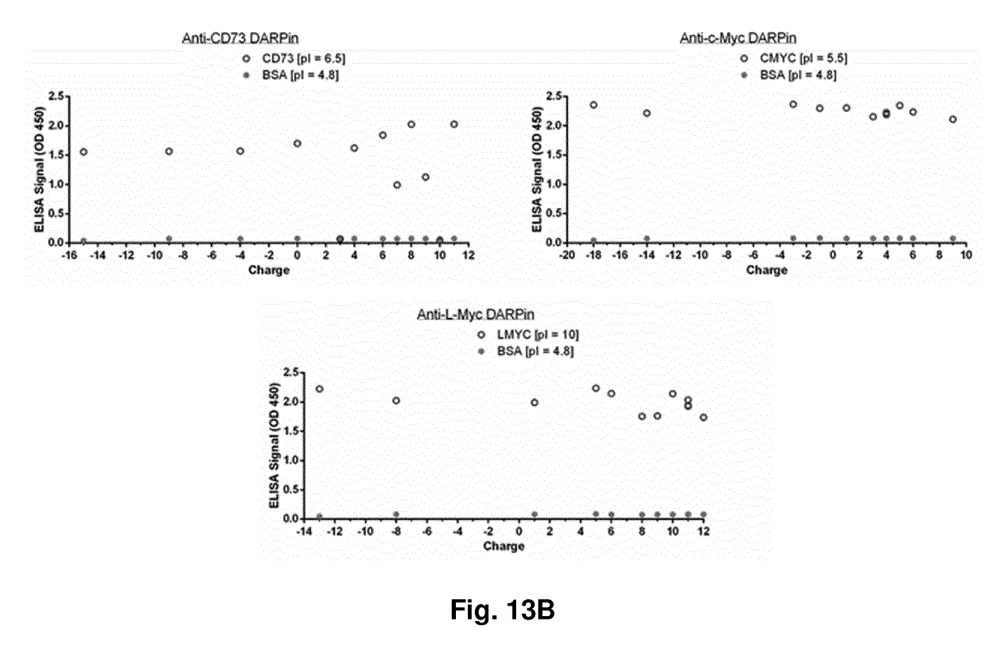

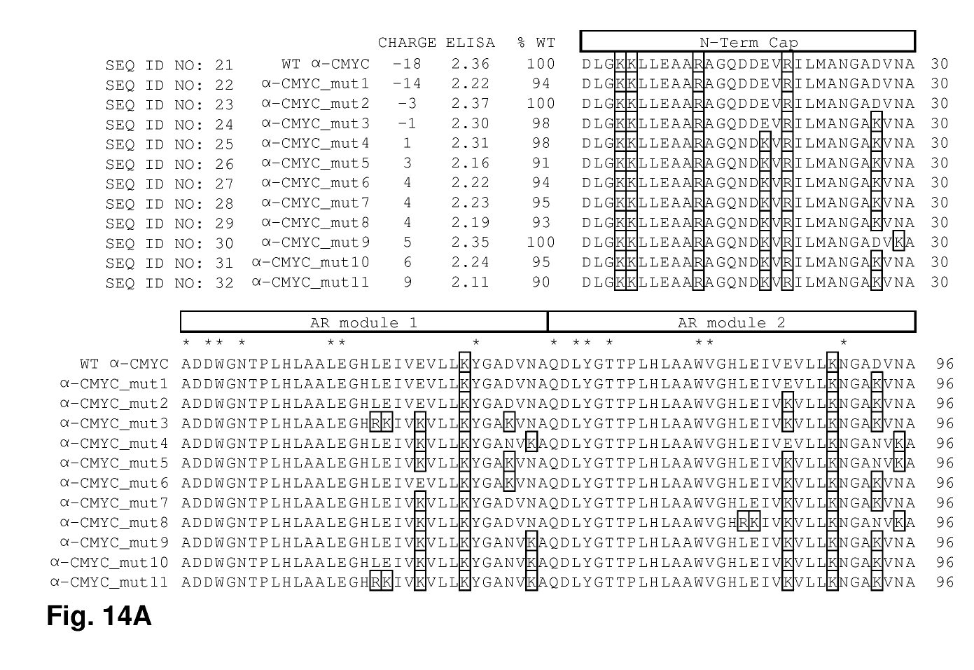

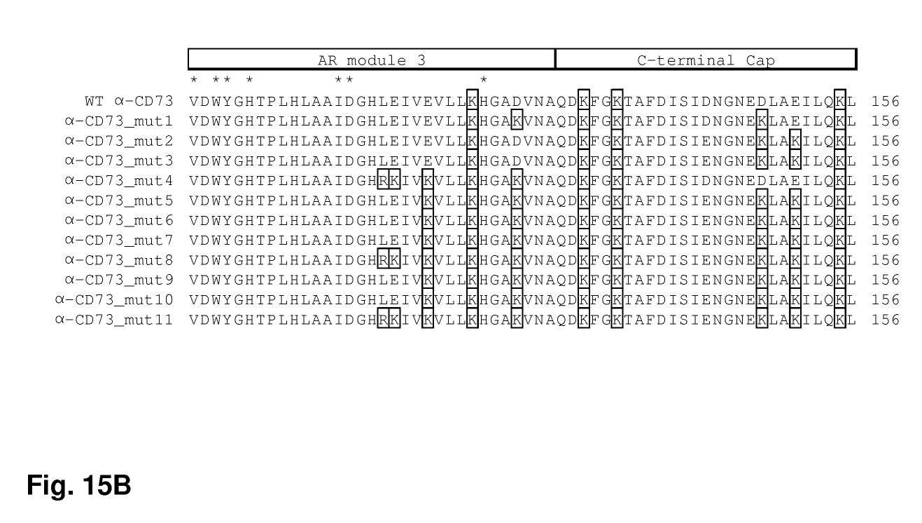

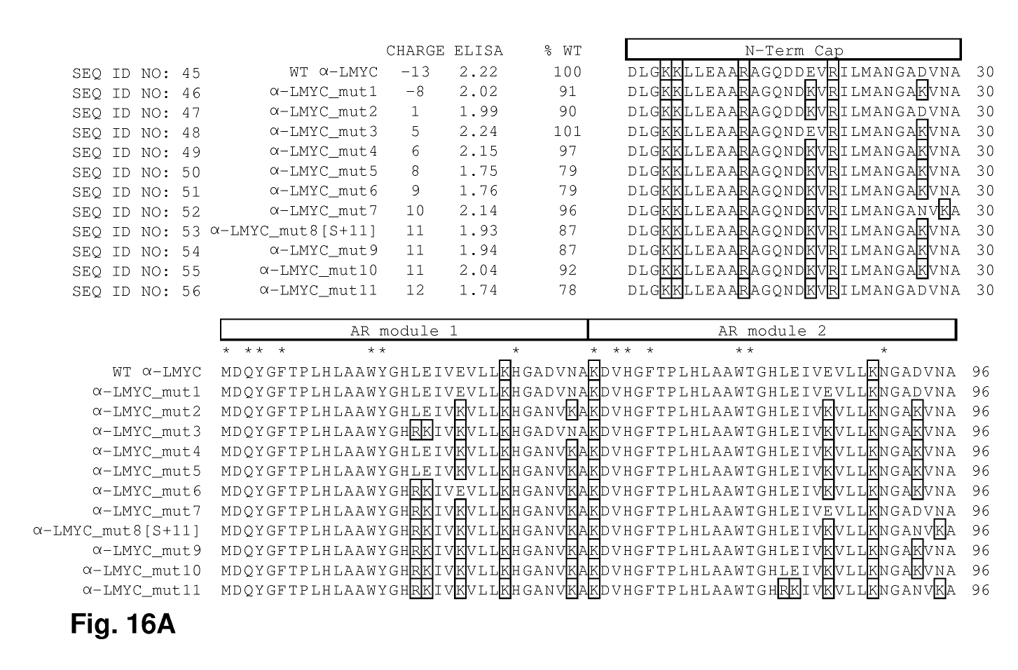

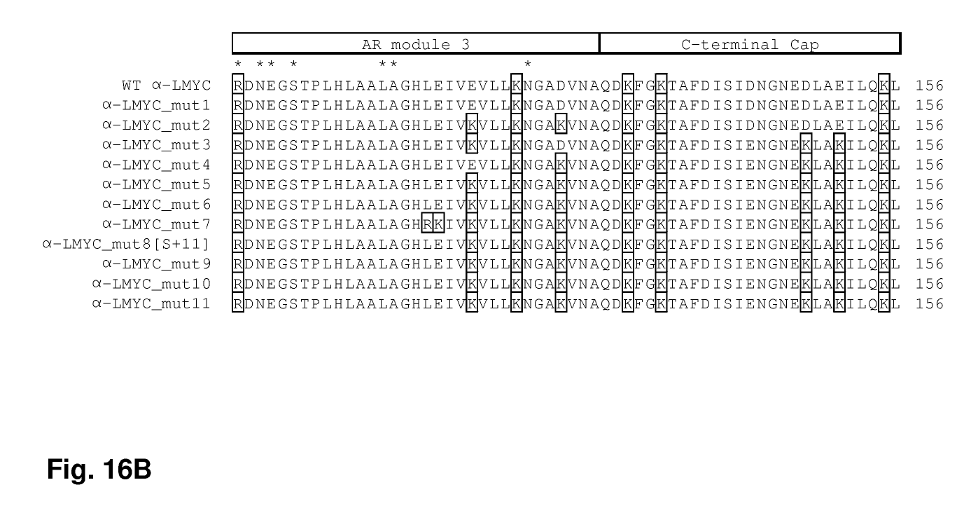

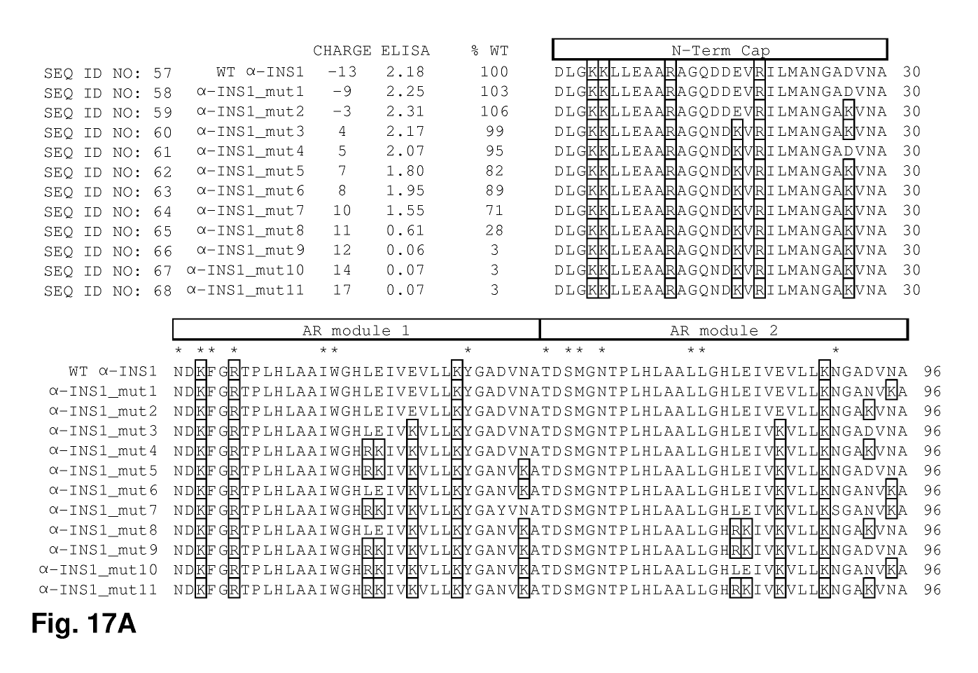

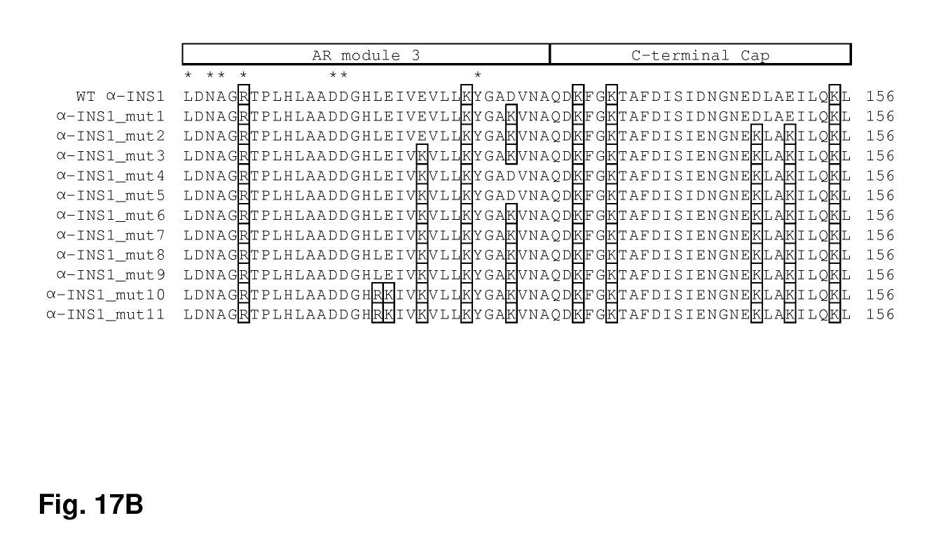

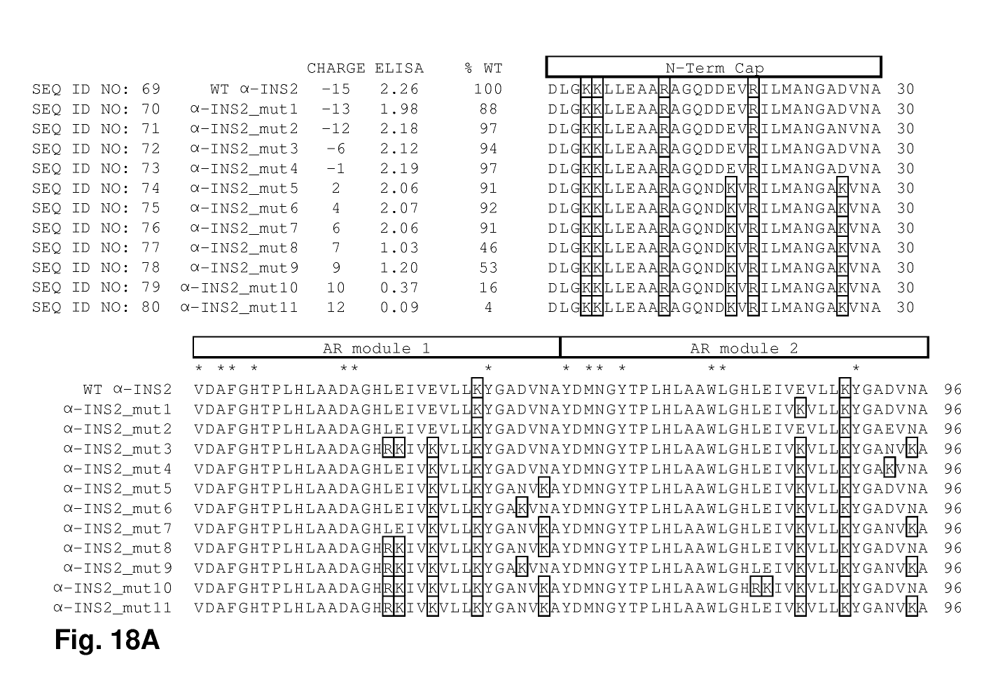

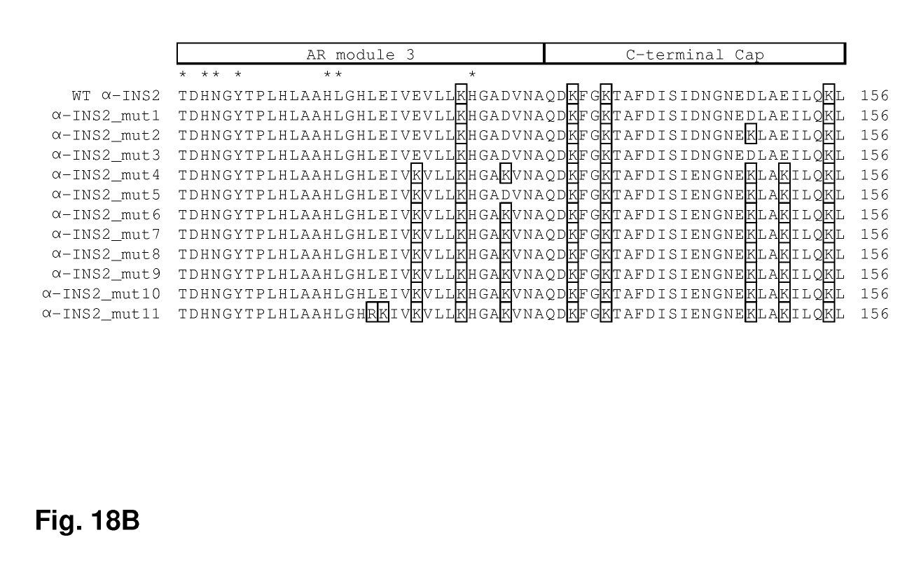

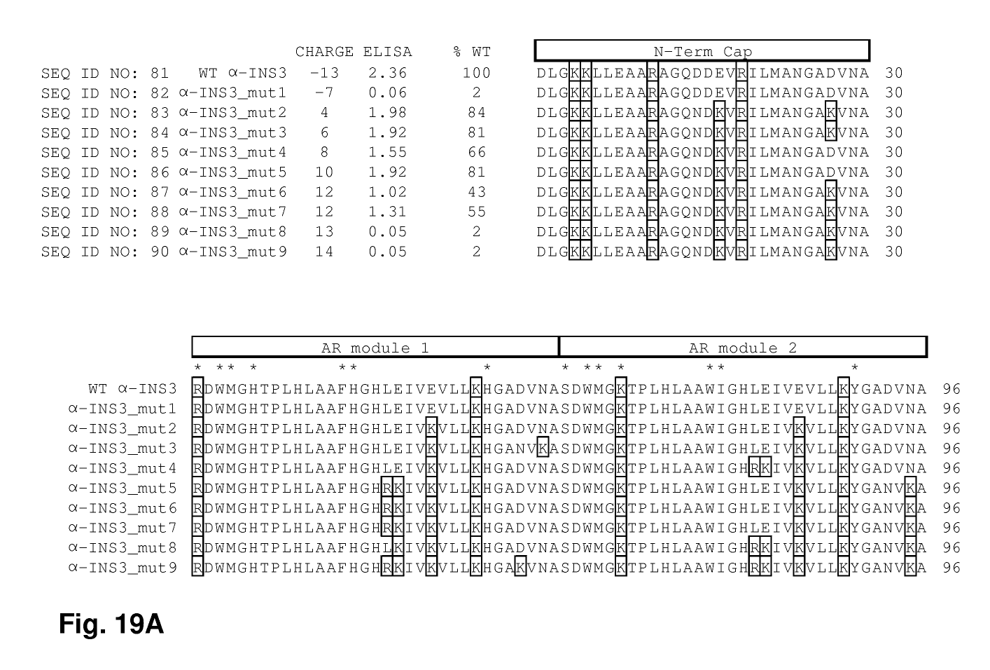

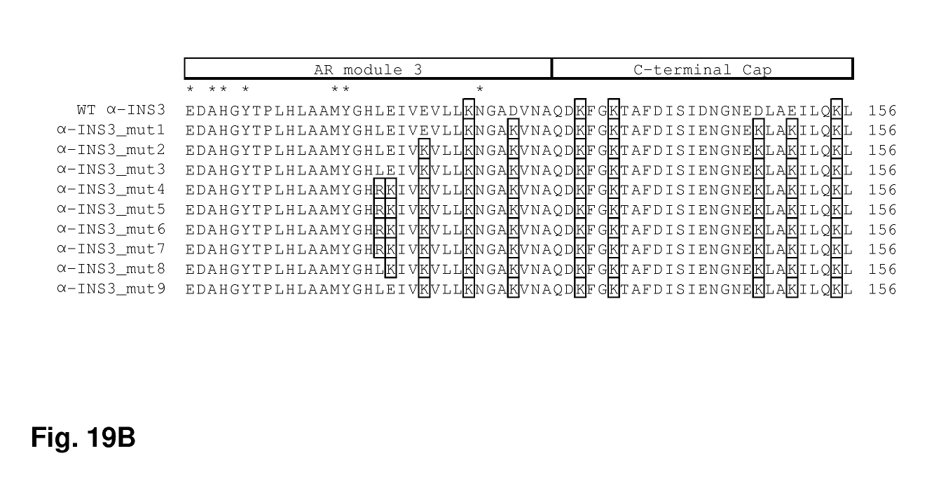

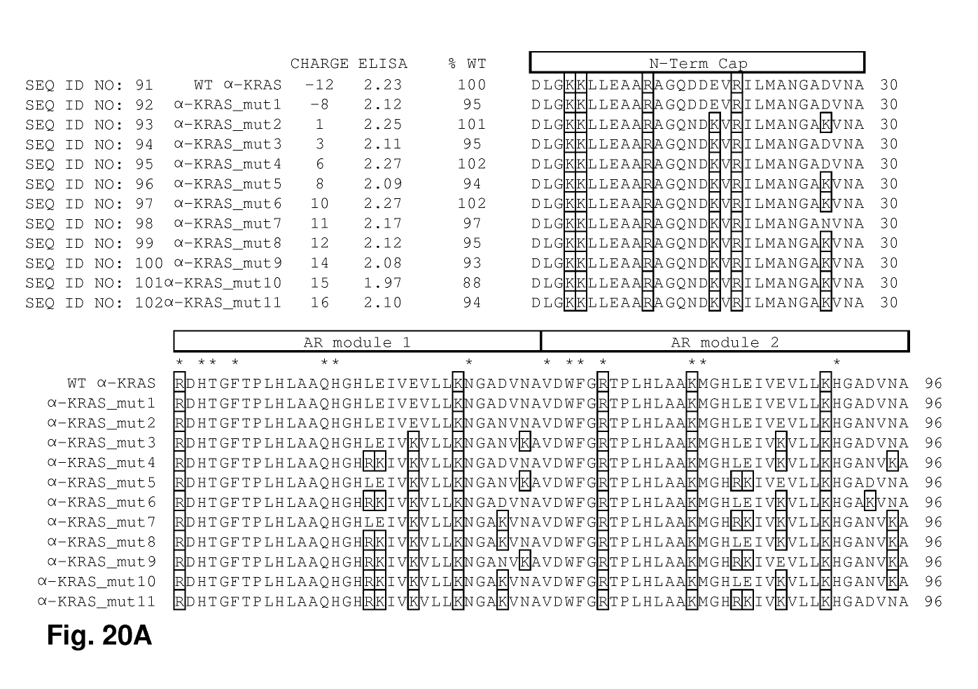

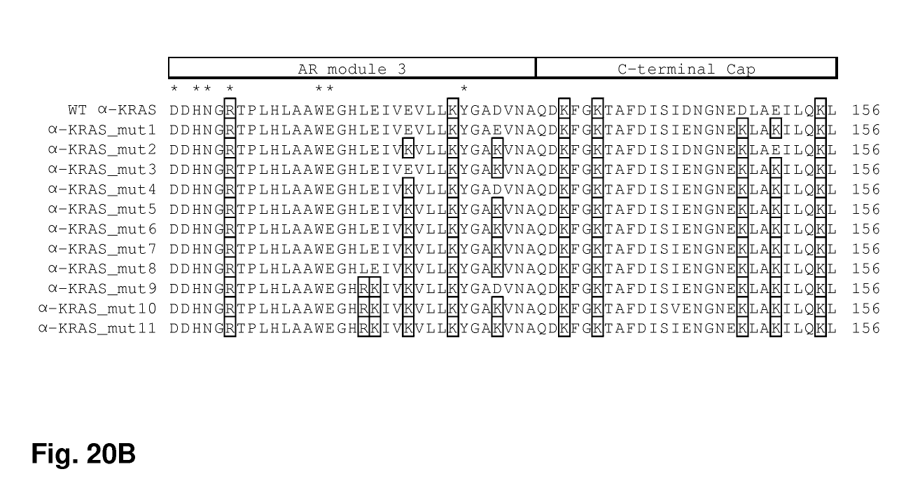

Using the methods described above numerous scDARPins variants were generated for seven different template DARPins having a starting charge of between -12 to -18. Positive charges were transferred to all seven of the DARPins generating scDARPins having a positive net charge of up to +16 without abolishing binding or specificity. The results are shown in FIGS. 13-21.

The substitutions and charge of representative scDARPin clones from each template DARPin are summarized in Table 4.

### Variant Residues

The 15 DARPins with the highest positive charge which retained >50% antigen binding activity (tested by ELISA) were aligned in order to determine positions which, in the majority of variants, could be positively charged without significantly reducing antigen binding (FIG. 21). The panel of 15 DARPins were derived from 5 different ‘parental’ DARPins against 4 different antigens (Insulin, KRAS, LMYC, CD73). A comparison of the original DARPin library design (net charge of −14, or −0.77/kDa) with a charged DARPin library design (net charge of +16, or 0.88/kDa), which incorporates positive charge at all the permissive sites, is also shown. The charged positions are also summarized in Table 4.

The present inventors suggest that the data presented herein are a good indicator for which residues to mutate in other DARPins. In particular, D15N, E17K, D27K, L48R, E49K, E52K, D6OK/N, N62K, E85K, D93K/N, E118K, D126K, D143E, D148K, and E151K are likely to lead to good results and may be readily combined. Numerous combinations may be quickly tested using the methods provided herein (e.g., wobble mutagenesis and/or use of specific primers to generate small libraries which are then screened for the desired binding activity).

TABLE 4
Substitutions in representative Supercharged DARPins

| | Pro. | Xₙ | | | | | | | | | |
|---|---|---|---|---|---|---|---|---|---|---|---|
| | rel. | rel. | | | | | | | | | |
| | SEQ | SEQ | | | | | | | | | |
| | ID | ID | | | | | | | | | |
| | NO: | NO: | | KRAS | KRAS | KRAS | LMYC | NS3 | KRAS | LMYC | CD73 |
| Sect. | 1 | 8 | WT | mut11 | mut10 | mut9 | mut11 | mut9 | mut8 | mut8 | mut11 |
| N-term | 15 | 1 | D | N | N | N | N | N | N | N | N |
| CAP | 17 | 2 | E | K | K | K | K | K | K | K | K |
| | 27 | 5 | D | K | K | K | — | K | K | K | K |
| AR | 48 | 6 | L | R | R | R | R | R | R | R | — |
| Module | 49 | 7 | E | K | K | K | K | K | K | K | — |
| 1 | 52 | 8 | E | K | K | K | K | K | K | K | K |
| | 60 | 9 | D | K | K | N | N | — | K | N | N |
| | 62 | 10 | N | — | — | K | K | — | — | K | — |
| AR | 81 | 11 | L | R | — | R | R | — | — | — | R |
| Module | 82 | 12 | E | K | — | K | K | — | — | — | K |
| 2 | 85 | 13 | E | K | K | — | K | K | K | K | K |
| | 93 | 14 | D | — | N | N | N | N | N | N | K |
| | 95 | 15 | N | — | K | K | K | K | K | K | — |
| AR | 114 | 16 | L | R | R | R | — | R | — | — | R |
| Module | 115 | 17 | E | K | K | K | — | K | — | — | K |
| 3 | 118 | 18 | E | K | K | K | K | K | K | K | K |
| | 126 | 19 | D | K | K | — | K | K | K | K | K |
| C-term | 143 | 21 | D | E | E | E | E | E | E | E | E |
| CAP | 148 | 23 | D | K | K | K | K | K | K | K | K |
| | 151 | 24 | E | K | K | K | K | K | K | K | K |
| Net Charge | | | | 16 | 15 | 14 | 12 | 12 | 12 | 11 | 11 |
| ~^{†}Charge per KDa | | | | 0.94 | 0.88 | 0.82 | 0.70 | 0.70 | 0.70 | 0.65 | 0.65 |
| | Pro. | Xₙ | | | | | | | | | |
| | rel. | rel. | | | | | | | | | |
| | SEQ | SEQ | | | | | | | | | |
| | ID | ID | | | | | | | | | |
| | NO: | NO: | | LMYC | LMYC | KRAS | LMYC | NS1 | NS3 | KRAS | NS2 |
| Sect. | 1 | 8 | WT | mut9 | mut10 | mut7 | mut7 | mut7 | mut6 | mut6 | mut9 |
| N-term | 15 | 1 | D | N | N | N | N | N | N | N | N |
| CAP | 17 | 2 | E | K | K | K | K | K | K | K | K |
| | 27 | 5 | D | K | K | N | N | K | — | K | K |
| AR | 48 | 6 | L | R | R | — | R | R | R | R | R |
| Module | 49 | 7 | E | K | K | — | K | K | K | K | K |
| 1 | 52 | 8 | E | K | K | K | K | K | K | K | K |
| | 60 | 9 | D | N | N | K | N | Y | — | — | K |
| | 62 | 10 | N | K | K | — | K | — | — | — | — |
| AR | 81 | 11 | L | — | — | R | — | — | — | — | — |
| Module | 82 | 12 | E | — | — | K | — | — | — | — | — |
| 2 | 85 | 13 | E | K | K | K | — | K | K | K | K |
| | 93 | 14 | D | K | K | N | — | N | N | K | N |
| | 95 | 15 | N | — | — | K | — | K | K | — | K |
| AR | 114 | 16 | L | — | — | — | R | — | R | — | — |
| Module | 115 | 17 | E | — | — | — | K | — | K | — | — |
| 3 | 118 | 18 | E | K | K | K | K | K | K | K | K |
| | 126 | 19 | D | K | K | K | K | K | K | K | K |
| C-term | 143 | 21 | D | E | E | E | E | E | E | E | E |
| CAP | 148 | 23 | D | K | K | K | K | K | K | K | K |
| | 151 | 24 | E | K | K | K | K | K | K | K | K |

| | | | | | | | | |
|---|---|---|---|---|---|---|---|---|
| Net Charge | 11 | 11 | 11 | 10 | 10 | 10 | 10 | 9 |
| ~^{†}Charge per KDa | 0.65 | 0.65 | 0.65 | 0.58 | 0.58 | 0.58 | 0.58 | 0.52 |

^{†}Calcuted assuming molecular weight of 17 KDa.

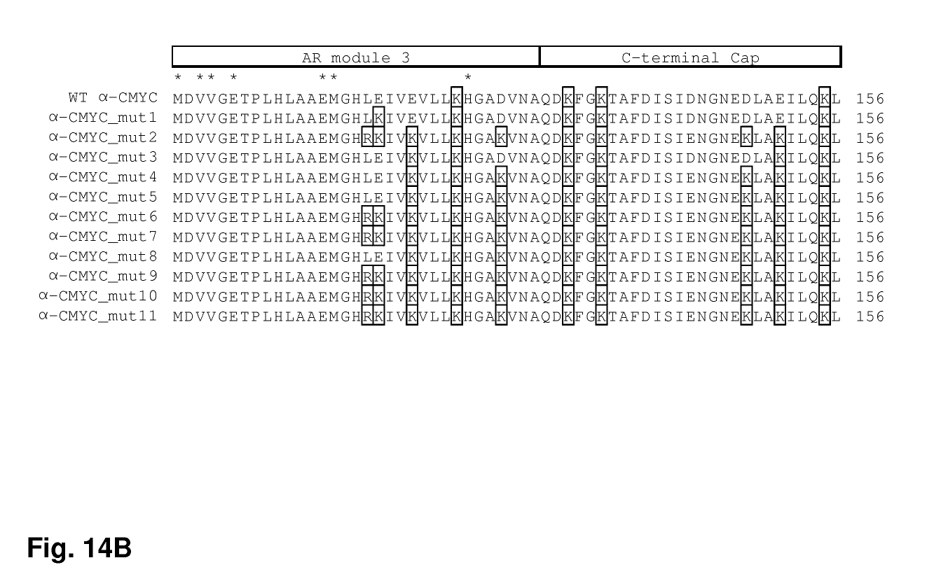

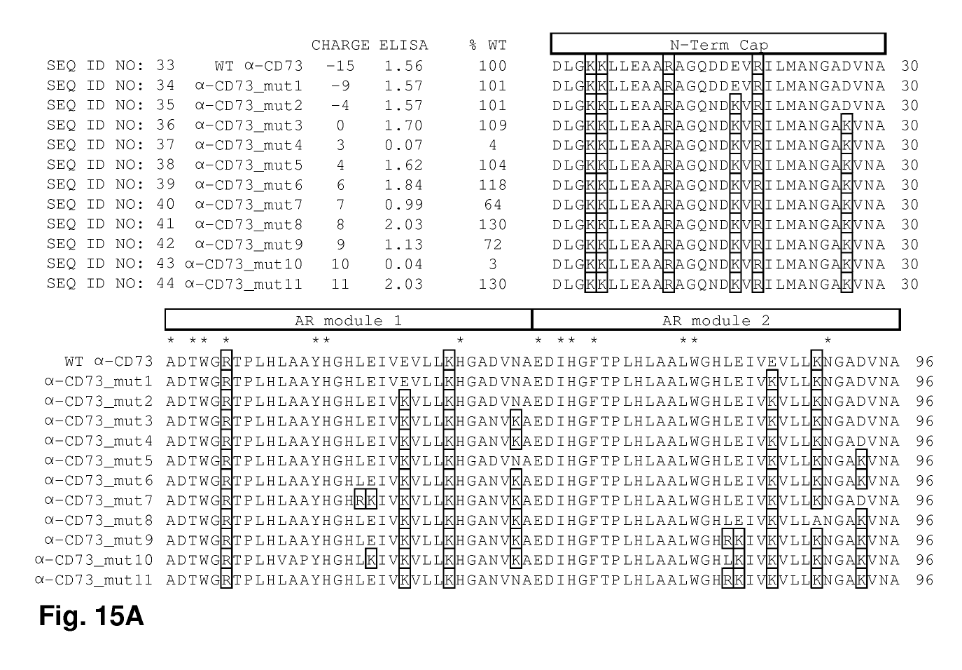

The present inventors have determined which positions in the DARPin scaffold are tolerant of being changed with a more positive amino acid. The results are shown in FIGS. 14-21 and summarized in Table 4.

### Results

The generation of a library of supercharged variants followed by charge selection is a promising method for identifying supercharged proteins. In this study several “supercharged” antigen specific DARPins were identified that retained antigen binding. The method also improves the theoretical ability of a supercharged protein to bind heparan sulphate on proteoglycans, as the selection has been performed on the homologous heparin.

The present inventors have determined which positions in the DARPin scaffold are tolerant of being changed with a more positive amino acid (FIGS. 14-21 and Table 4 above).

Few of the scDARPins identified had a charge above +0.75 charges/kDa, which is described as the minimum requirement for efficient internalization (Cronican et al., 2011). However, increases of =+22 to +25 charges were routinely generated and represent a significant change in the electrostatic potential of the DARPins.

The method of the invention is applicable to supercharging other DARPins. The task of supercharging would further be eased by finding template DARPins with a higher net charge. All the sequenced DARPins (>300) in the initial L-Myc selection had a net charge of −13 or below.

In alternative methods the generation of a new library of supercharged variants might not even be necessary to supercharge DARPins that bind other antigens. The most promising mutations identified have been directly applied to other DARPins, thereby creating the supercharging. Ideally a library with a supercharged DARPin framework as template and randomised residues in the variable positions could be created, which would enable the direct identification of a scDARPin against any antigen. Such an approach could create DARPins against many different intracellular antigens in a relatively short time.

### Example 2

### Purification of Supercharged DARPins (scDARPins)

Expression and purification of 18 scDARPins with retained L-Myc binding was investigated.

Several studies have described the effectiveness of using the pQE-30 vector (Qiagen) for high yield expression of DARPins (Binz et al., 2003, Interlandi et al., 2008). This vector is used in combination with M15 pREP 4 cells for expression of both the template DARPin and the supercharged versions.

### Expression

The pQE-30 vector contains 6 histidines at the N-terminus and the gene of interest can be cloned into the vector between the BamHI and HindIII restriction site. The vector has ampicillin resistance and a lac operon (lacO), but no lac repressor (lacl). The lacl gene is instead encoded in the pREP4 plasmid that also contains kanamycin resistance in the M15 cells. The repression can be removed by addition of Isopropyl β-D-1-thiogalactopyranoside (IPTG), which leads to protein expression.

For expression all DARPins were sub-cloned from the pCANTAB6 vector used during the phage display to a pQE-30 vector (Qiagen). The pQE-30 vector is used for cytoplasmic expression and contains a 6xHis-tag for purification.

BamHI and HindIII restriction sites and a cysteine at the C-terminal were inserted using PCR primers and Phusion HF Master Mix (NEB). The scDARPin inserts and the pQE-30 vector were then digested with BamHI HF and HindIII HF restriction enzymes (NEB) and gel purified with High pure PCR product purification kit (Roche, Agarose purification protocol). Inserts and pQE-30 vector were ligated with T4 DNA ligase (NEB) and transformed into chemically competent M15 pREP4 cells.

The scDARPins in pQE-30 were transformed into chemically competent M15 pREP4 cells. 1 colony was picked into 30 ml 2×TYAGK media and grown overnight at 37° C. (280 rpm). The culture was added to 400 ml 2×TYAK media and grown at 37° C. (280 rpm) for 1-1.5 h before addition of IPTG (1 mM). The culture was grown a further 4-4.5 h before it was centrifuged in a Sorvall centrifuge at 10,000 rpm (15 min, 5° C.). The supernatant was discarded and the cell pellet frozen at −20° C.

The pellet was thawed and 15 ml BugBuster (Novagen) with 25 U/ml Benzonase nuclease (Novagen) and 0.6 M NaCl (scDARPins) or 1.3 M NaCl (+36 GFP) was added and incubated for 25 min. The mixture was centrifuged at 4000 rpm (15 min, 5° C.).

The supernatant was separated from the cell debris before his-tag purification in columns containing Nickel Sepharose 6 fast flow resin (GE healthcare). The proteins were eluted with elution buffer 1 (50 mM Tris, 300mM NaCl, 400 mM imidazole, pH 8.0) or for the supercharged proteins by elution buffer 2 (50 mM Tris, 1.0 M NaCl, 400 mM imidazole, pH 8.0). Buffer exchange was performed with NAP-10 columns (GE Healthcare) into PBS. Determination of protein concentrations were performed by bicinchoninic acid (BCA) assay (Thermo Scientific).

Ultrafiltration was performed on all samples used in cell assays by Ultrafree-MC 0.22 μM centrifugal filters (Millipore).

For small scale test experiments (5-50 ml cultures) 200 μl BugBuster with 25 U/ml Benzonase and varying NaCl concentrations were used in eppendorf tubes per 1 ml of culture volume. Centrifugation was performed in a microcentrifuge (13,000 rpm, 10 min). The supernatant (soluble fraction) was separated from the cell debris, and the cell debris was re-suspended in the same volume of BugBuster (insoluble fraction). Sodium dodecyl sulfate polyacrylamide gel electrophoresis (SDS-PAGE) on a 4-12% Bis-Tris mini gel was performed according to the manufactures recommendations (Invitrogen) with Instant Blue (Novexin) staining.

To create scDARPins with a higher net charge, whole plasmid site-directed mutagenesis was used with the S+11 DARPin in pQE-30 as a template. 2 of the variable residues were changed to Alanine (Y34A, F36A), to create similar, but non-binding versions of the scDARPins. Miniprep purification was performed on 5 ml of M15 pREP4 cells containing the relevant scDARPin in a pQE-30 vector according to the manufactures instruction (Qiagen). 100 ng of the vector was used in a PCR reaction with 1 ng of each forward and reverse mutagenic oligoes and Phusion HF Master Mix (NEB). After the reaction the parent DNA was digested with Dpnl enzyme (NEB). The mutagenic plasmids were then transformed into chemically competent M15 pREP4 cells followed by sequence analysis (DNA chemistry, MedImmune) to confirm the mutations.

To enable detection of scDARPins internalised within cells in flow cytometry experiments, a fluorophore was conjugated to the cysteine at the C-terminus. The DARPins were reduced in a 5 fold molar excess of tris(2- carboxyethyl)phosphine (TCEP) for 45 min at 37° C. Then 1 mg/ml Alexa Fluor 488 C5 maleimide (Invitrogen) in dimethylformamide (DMF) were added in a 3 fold molar excess and incubated for 1.5 h at 37° C. To remove excess dye either Zeba Spin desalting columns (7 MWCO, Thermo Scientific) or Slide-A-lyzer overnight dialysis in PBS buffer (3 kDa MWCO, Thermo Scientific) was used.

To determine labeling efficiency, matrix-assisted laser desorption/ionisation time of flight mass spectrometry (MALDI-TOF) was performed. 1 μl of labeled protein were mixed with 9 μl 10 mg/ml α-cyano-4-hydroxycinnamic acid (CHCA) matrix and dried. MALDI-TOF was then performed on the dried matrix.

### Expression of Soluble Protein

The present inventors' initial experience with both scDARPins and +36 GFP were that they could only be found as insoluble proteins. The template DARPin and scGFP had both expressed soluble at very high levels (3.2 and 14.0 mg purified protein per liter of culture volume). To better understand the problem it was decided to focus on the expression +36 GFP as this protein previously had been expressed (Lawrence et al., 2007) and its fluorescence made it easy to determine its solubility.

It was surprisingly found that addition of a high NaCl concentration (1.0 M NaCl, 10 mM Tris pH 7.5) to BugBuster (with benzonase) released some of +36 GFP to the soluble fraction. A further increase to 1.2 M NaCl or above released almost all of the +36 GFP from the insoluble fraction.

Addition of 1.67 M NaCl to the BugBuster during the lysis step enabled 8 of the 9 scDARPins to be observed in the soluble fraction. For two of the scDARPins it was further shown that 0.6 M of NaCl was enough to release the supercharged proteins into the soluble fraction.

### Purification of Higher Positively-Charged DARPins

The S+11 scDARPin was expressed and purified at only half the amount of the template DARPin, although it still expressed large amounts of DARPin. To test if higher charge would decrease the expression further two new scDARPins were designed.

The design was based on the results obtained during the phage display selection on heparin. As some residues were enriched to positive residues more than others, these would be more likely to increase the binding affinity to heparin, and therefore increased internalization by endocytosis after binding to heparan sulphate proteoglycans on the cell surface.

The L-Myc scDARPin with the highest charge net charge, the S+11 scDARPin, was chosen as template for the new mutations. This scDARPin already had most of the mutations that were enriched most, but four residues which were increased between 14 and 31 percentage points were still good candidates. From the modeling of the S+11 scDARPin it was also observed that two of these, the E82 and E115, were disrupting an otherwise very positively-charged area on one side of the protein. N128K and E147K were decreased 23 and 14%, during L-Myc selection and phage rescue (FIG. 10), and could potentially cause a decrease in binding, stability or expression.

The 3 repeat regions had the same mutations, except for AR1, which had one additional mutation (L48R). It is possible that the same mutation could be beneficial in the 2 other AR's (this was already shown in the transfer of charge experiment). From a combination of these six positions three scDARPins were designed with a net charge of +14, +18 and +19, respectively.

Compared to the previous scDARPins slightly more NaCl was needed to release the S+14; and 1.0 M NaCl was required to release the S+18. The dimers, presumably linked through cysteine interactions, were however only released at 1.5 M NaCl.

The scDARPins were purified by his-tag chromatography. After initial purifications could not elute the scDARPins properly, it was found that a high concentration of NaCl in the elution buffer was needed to elute the proteins. With this approach an acceptable S+11 scDARPin yield of 48% of the S-13 tDARPin were produced.

It will be understood that KCl may be used instead of NaCl. Although NaCl or KCl are the most commonly used salts during protein purification, others such as CaCl₂ or MgCl₂ could also be considered as substitutes (Guide to Protein Purification, Edited by Murray P. Deutscher, Methods in Enzymology, Academic Press). These types of salts would be are commonly used, but others may also be known to those skilled in the art of protein purification.

### Results

Supercharging the DARPins resulted in some decrease in the yields of soluble protein. The decrease came partly from reduced protein expression and partly because of increased purification losses. Whether this decrease is caused by the charge or by the fact that the higher charge is due to more mutations, which generally increases the chance of disrupting the stable structure of the template DARPin is unknown. As the template DARPin expressed at very high levels, the decrease in yield for scDARPins were not a problem for those clones selected during the L-Myc and heparin panning process.

It was also found that NaCl was required to release the scDARPins during lysis and purification. The salt likely disrupted non-specific binding to negatively charged molecules, such as DNA, and greatly enhanced the yield.

### Example 3

### Functionality of Supercharged DARPins (scDARPins)

### Internalisation

The internalization of +36 GFP has been determined to be through endocytosis (McNaughton et al., 2009, Thompson et al., 2012). Endocytosis is used to take up nutrients from the surroundings and to regulate cell surface receptors. Endocytosis is also a common way for pathogens to gain entry to cells (Mercer et al., 2010).

There are several routes of endocytosis, where different receptors and other proteins are involved. One of the best described endocytic pathways (Mukherjee et al., 1997), and the one of most importance to +36 GFP (Thompson et al., 2012), is clathrin dependent endocytosis. This type of endocytosis occurs when certain receptors come into contact with their ligands (Doherty and McMahon, 2009). The coat protein clathrin is involved in getting the membrane to bud inwards to form a vesicle within the cell.

There is strong evidence that the receptors for internalization of +36 GFP are sulphated proteoglycans. In a mutant cell line without the ability to synthesize proteoglycans or when cells are treated with sodium chlorate that inhibits the formation of sulfated proteoglycans, the internalization of +36 GFP is completely blocked (McNaughton et al., 2009).

Once the supercharged proteins have been internalized by endocytosis, the vesicles will be part of the endosomal network. Generally the vesicles will become early endosomes within 2 minutes of internalization (Mercer et al., 2010). Each stage of the maturation process is categorized by a number of surface markers and sorting proteins, with increased acidic levels at each stage. The endosomes will generally become maturing and late endosomes within 10-12 minutes, and then fuse with lysosomes, with a pH of 4.5-5, within 30 minutes (Mercer et al., 2010) or 2 h (Blanchette et al., 2009) depending on cell type. Alternative routes include recycling the vesicles back to the outer membrane of the cells.

Several other positively charged proteins can also be internalized, most notably cell penetrating peptides and certain members of the ribonuclease (RNase) family.

Many of the cell penetrating peptides are derived from viruses that use a series of positively charged amino acids to gain access to mammalian cells (Dietz and Bahr, 2004). One example is the transduction domain of trans-activating transcriptional activator (Tat) from the HIV-1 virus. Of the 11 residues in the transduction domain of Tat 6 are arginines and 2 are lysines (Kaplan et al., 2005). Tat and similar peptides can be fused to proteins and thereby facilitating internalization (Kaplan et al., 2005, Fuchs and Raines, 2004). The cell penetrating peptides are internalized by a range of different endocytotic mechanisms, while Tat is only internalized by macropinocytosis (Wadia et al., 2004). +36 GFP has however been shown to significantly improve internalization of the mCherry-protein, compared to Tat, with up to 100 fold (Cronican et al., 2010).

Several proteins from the RNase family have several similarities with supercharged GFP although their overall positive net charge is only +2-5 (<0.5 charges/kDa). Pancreatic ribonuclease A (RNase A) is internalized by both clathrin dependent endocytosis and macropinocytosis (Chao and Raines, 2011). It exerts its toxic activity by catalyzing the degradation of cellular RNA. Another member of the family is Onconase. Onconase is internalized in a clathrin, but not proteoglycan dependent manner (Chao et al., 2010). 12 lysines and 3 arginines are responsible for the positive charge of Onconase. By changing 10 of the lysines to arginines (R-Onconase) the heparin affinity was markedly increased and internalization was increased 3-fold (Sundlass and Raines, 2011). The cytotoxicity of R-Onconase was however not increased due to increased proteolytic degradation and possibly alterations in the internalization mechanism.

### Flow Cytometry

Flow cytometry is commonly used for determining internalization of molecules such as cell penetrating peptides and +36 GFP. The internalization was determined with Alexa Flour 488 attached to the tested DARPins, for detection. Another widely used method is confocal microscopy, although the present inventors have used a high throughput image flow cytometry method since this gives the advantage of analyzing thousands of cells.

HeLa cells (ATCC) were grown in T175 flasks. The cells were seeded at 5.5×105 per well in 24 well plates and incubated at 37° C., 5% CO2. The next day the cells were washed once with PBS and 500 μI Minimum Essential medium (MEM) containing 50-500 μM supercharged protein. The concentrations of labeled protein were determined by the absorbance at 495 nm (cAlexa Flour 488 =71,000 M-1 cm-1).

The cells were then incubated for 4 h at 37° C., 5% CO2. Then the cells were washed 3 times for 1 minute with cold PBS containing 20 μ/ml heparin. Negatively charged heparin was reported by McNaughton et al. (2009) to be required for efficient surface bound +36 GFP removal. For time dependent studies 500 μl MEM, 10% fetal bovine serum (FBS), 1% Non-essential amino acids (NEAA) was added to each well and incubated at 37° C., 5% CO2 until used or treated directly with accutase (PAA).

100 μl accutase were added and incubated until the adherent cells were released. The accutase was neutralized with 200 μl MEM, 10% FBS, 1% NEAA and the mixture was transferred to a 96 well U-bottom plate. The plate was centrifuged at 1200 rpm for 3 min and the supernatant was discarded. The cells were re-suspended in 200 μl PBS for flow cytometry or 50 μl PBS for image flow cytometry.

For heparin wash efficiency tests the cells were instead incubated at 4° C. for 1 hour with pre-cooled media.

For determination of intracellular localization LysoTracker Red (invitrogen) and Hoechst 33342 (Invitrogen) were used. LysoTracker was added to the cells to a concentration of 50 mM 2 h before the end of the incubation. Hoechst was diluted to 2μg/ml in accutase and incubated with the cells during the accutase treatment step.

The cells were either analyzed by FACSCanto II flow cytometer (BD) or image flow cytometry by ImageStreamX Mark II (Amnis). In flow cytometry Alexa Fluor 488 and +36 GFP are excited by a 488 nm laser and detected by the FITC filter (530 nm). In image flow cytometry Alexa Fluor 488 and +36 GFP are excited by a 488 nm laser and detected by channel 2 (480-560 nm), LysoTracker is exited at 561 nm and detected in channel 4 (595-642 nm) Hoechst is excited by 405 nm laser and detected by channel 7 (430-505 nm).

The data were analyzed by FlowJo (version 7.6.1) and Ideas (version 5.0), respectively.

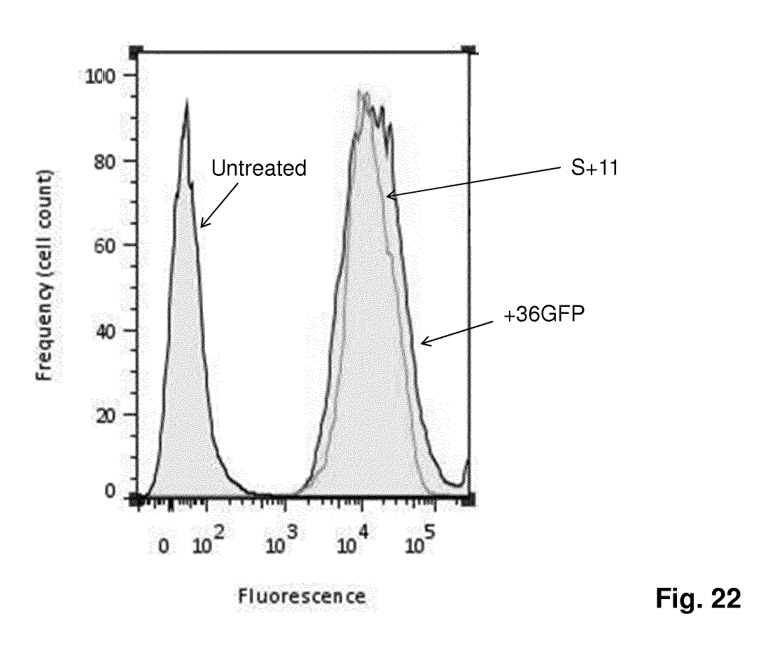

Internalization of L-Myc DARPins was determined by flow cytometry in HeLa cells. HeLa cells have not been reported to be sensitive to L-Myc inhibition, but their simple handling and ability to internalize +36 GFP, made them attractive for initial testing. A very significant increase in fluorescence was observed when the cells were treated with Alexa Fluor 488 labeled S+11 L-Myc scDARPin (FIG. 22). Cells were also treated with +36 GFP and showed a similar shift in fluorescence, although the intensities between the fluorescence are not comparable.

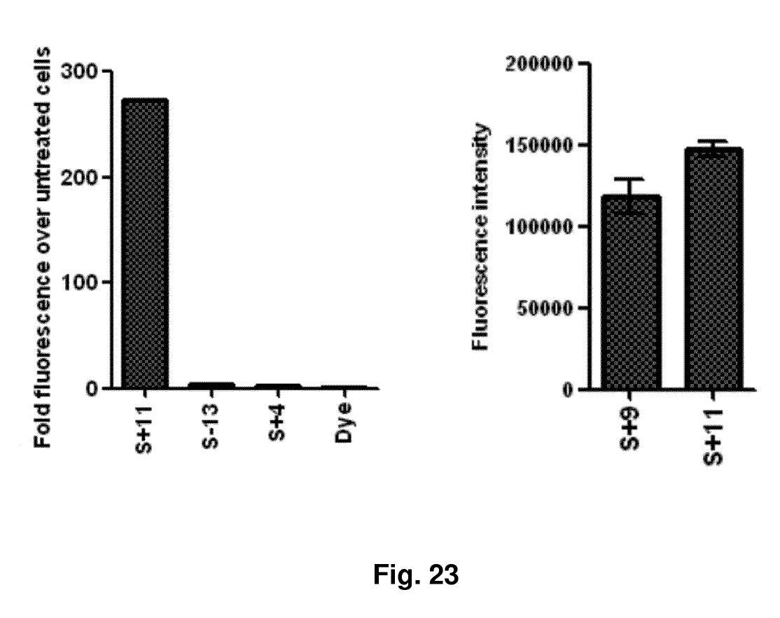

To confirm that the shift in fluorescence was caused by the charge, cells were also treated with the template L-Myc DARPin with a net charge of -13. These cells did not have a similar increase in fluorescence, although a 3 fold increase compared to untreated cells were observed at 200 nM. This change was however considered insignificant when compared to the 290 fold increase for S+11 scDARPin. The S+4 scDARPin did not internalize (FIG. 23), while the S+9 was internalized about 80% of that of S+11 scDARPin at 50 nM. Due to lower yields of the S+14 and S+18 scDARPins these were not tested by flow cytometry, and instead only tested for functionality.

To confirm that any non-removed and non-conjugated dye could not internalize on its own, cells were incubated with 200 nM of Alexa Fluor 488. The fluorescent increase for these cells was less than 2% of S+11 scDARPin, and confirms that the measured fluorescent signals come from Alexa Fluor 488 conjugated to scDARPins (FIG. 23).

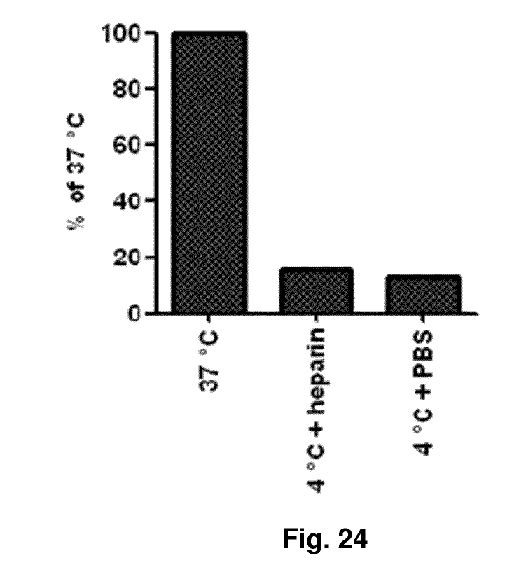

The flow cytometry data proves that the supercharged DARPins are in close proximity to the cells. To eliminate the possibility that the scDARPins are bound to the surface, the cells were washed 3 times with heparin and treated with proteases (accutase). As endocytosis is an energy-dependent process, incubation at 4° C. before and during treatment with scDARPins should prevent uptake. By this approach the internalization was lowered to 13-16% of the internalization at 37° C. (FIG. 24). The addition of heparin did not decrease the fluorescent signal compared to without heparin, indicating that the remaining signal was internalized and not bound to the surface.

To further confirm that the scDARPins were inside of the cells, image flow cytometry was applied. Each individual cell is photographed and analyzed for fluorescence, which enables the high throughput localization of fluorophores. The data were generated by capturing 10,000 cells and then gating images adequately in focus, which each contain one cell.

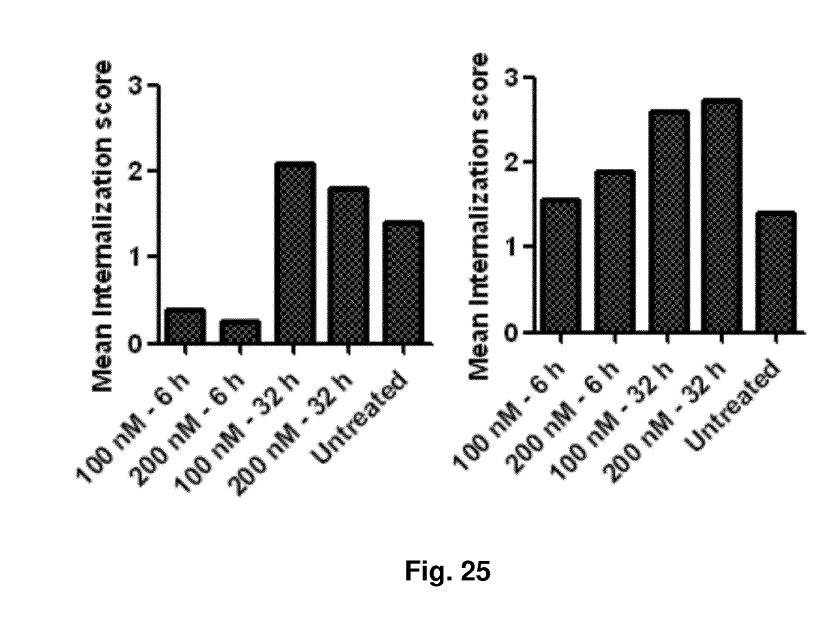

Surface bound markers are characterized as a ring around the cells. Therefore was the analysis for measuring internalization performed by measuring the fluorescence outside and in the 5 outermost pixels in the cells and comparing it to the fluorescence in the middle of the cells. This analysis revealed a high degree of internalization for the S+11 scDARPin, with positive values indicating internalization (FIG. 25).

The supercharged DARPins with a net charge of +9 and +11, corresponding to about +0.5 and +0.6 net charges/kDa were both shown to be effectively internalized in HeLa cells. As +36 GFP has been shown to be internalized in 7 different cell lines it is therefore expected that the supercharged DARPins also can internalize in several different cell types.

The scDARPins are effectively a new type of protein, with the ability to internalize in mammalian cells.

1. A Designed Ankyrin Repeat Protein (DARPin) that specifically binds to an antigen, the DARPin comprising an N-terminal cap section, at least two Ankyrin Repeat (AR) module sections, and a C-terminal cap section, characterised in that the DARPin has a charge that is less negative than the DARPin of SEQ ID NO: 1, excluding the charge contribution of the variable antigen-binding residues.

2. A DARPin according to claim 1, wherein the DARPin has a neutral net charge.

3. A DARPin according to claim 1, wherein the DARPin has a positive net charge.

4. A DARPin according the claim 3 characterised in that the DARPin is capable of internalising into a cell.

5. A DARPin according to any one of the preceding claims, characterised in that it binds an intracellular antigen.

6. A DARPin according any one of the preceding claims, characterised in that the N-terminal cap section comprises SEQ ID NO:5, or optionally consists of SEQ ID NO: 5.

7. A DARPin according to any one of the preceding claims, characterised in that each AR module section comprises SEQ ID NO: 6, or optionally consists of SEQ ID NO: 6.

8. A DARPin according to any one of the preceding claims, characterised in that the C-terminal cap section comprises SEQ ID NO: 7, or optionally consists of SEQ ID NO: 7.

9. A DARPin according to any one of the preceding claims, characterised in that it exhibits at least a 10-fold higher, optionally at least a 100-fold higher, mean fluorescence index (MFI) as measured by flow cytometry, by comparison with a control.

10. A DARPin according to any one of the preceding claims, characterised in that it has three AR repeats.

11. A DARPin according to claim 10, characterised in that it has an amino acid substitution at one or more of amino acid residues D15, E17, 120, G25, D27, L48, E49, E52, D60, N62, L81, E82, E85, D93, N95, L114, E115, E118, D126, N128, D143, E147, D148 and/or E151, numbered relative to SEQ ID NO: 1.

12. A DARPin according to claim 10, characterised in that it has the amino acid sequence of SEQ ID NO: 8.

13. A DARPin according to claim 11, characterised in that it has one or more of the following substitutions: D15E/K/N, E17K, 120R, G25R, D27E/K/N, L48R, E49K, E52K, D60E/K/N, N62K, L81R, E82K, E85K, D93E/K/N, N95K, L114R, E115K, E118K, D126E/K/N, N128K, D143E/K/N, E147K, D148E/K/N and/or E151K, numbered relative to SEQ ID NO: 1.

14. A DARPin according to claim 11 or claim 13, characterised in that it has the mutations D15N, E17K, D27K, L48R, E49K, E52K, D6ON, N62K, E85K, D93N, N95K, E118K, D126K, D143E, D148K, E151K, numbered relative to SEQ ID NO:

15. A DARPin according to claim 12, characterised in that: X₁, X₉, and X₁₄ are N; X₂, X₅, X₇, X₈, X₁₀, X₁₃, X₁₅, X₁₈, X₁₉, X₂₃, and X₂₄ are K; X₃ is I; X₄ is G; X₆ is R; X₁₁, X₁₆, X₂₅, and X₂₆ are L; and X₁₂, X₁₇, X₂₁, and X₂₂ are E.

16. A DARPin according to any one of the preceding claims, characterised in that the positive net charge is less than +0.75/KDa, optionally characterised in that the positive net charge is less than +0.6/KDa.

17. A DARPin according to any one of the preceding claims, characterised in that the positive net charge is at least +0.60/KDa.

18. A DARPin according to any one of the preceding claims, characterised in that the positive net charge is at least +0.5/KDa.

19. A method of making a Designed Ankyrin Repeat Protein (DARPin) capable of (i) binding an antigen; and (ii) crossing the membrane of a cell, the method comprising:
a) generating a library of DARPins;
b) carrying out a first selection using the antigen;
c) carrying out a second selection using a negatively charged reagent;
d) eluting the DARPins; and
e) purifying the DARPins.

20. The method of claim 19, characterised in that the antigen is an intracellular antigen.

21. The method of claim 19 or claim 20, characterised in that step (b) is carried out by a method selected from the list consisting of: phage display, yeast display, and ribosome display.

22. The method of any one of claims 19 to 21, characterised in that step (b) is carried out using the intracellular antigen alone.

23. The method of any one of claims 19 to 22, characterised in that step (b) is carried out using the intracellular antigen and a negatively charged reagent.

24. The method of any one of claims 19 to 23, characterised in that the negatively charged reagent is Heparin.

25. The method of any one of claims 19 to 23 characterised in that the negatively charged reagent is selected from the list consisting of DNA, a negatively charged protein, an anionic liquid, a negatively charged membrane, or a negatively charged resin.

26. The method of any one of claims 19 to 23, characterised in that purification step (e) is carried out in the presence of a salt buffer.

27. The method of claim 26, characterised in that purification step (e) is carried out in the presence of NaCl or KCl.

28. The method of any one of claims 19 to 27, characterised in that the library of DARPins each comprise the amino acid sequence of SEQ ID NO: 6.

29. The method of claim 28, characterised in that the library of DARPins each comprise the amino acid sequence of SEQ ID NO: 5 and or SEQ ID NO: 7.

30. The method of any one of claims 19 to 29, characterised in that the library of DARPins each comprise the amino acid sequence of SEQ ID NO: 8, 9, 10 or 11.

31. A DARPin produced by the method of any one of claims 19 to 30.

32. A DARPin according to any one of claims 1 to 18, or claim 31, characterised in that it binds to its antigen with a K_{D} of 10⁻⁶ M or lower, optionally wherein it binds to its antigen with a K_{D} of 1 nanoMolar (nM) or lower.

33. A DARPin according to any one of claims 1 to 18, or claim 31 or 32, characterised in that it binds to its antigen with a potency (EC₅₀) of be less than 100 nanoMolar, optionally with a potency (EC₅₀) of 1-100 nM.

34. A DARPin library comprising a plurality of DARPins each comprising a DARPin framework sequence having an amino acid sequence according to SEQ ID NO: 8, wherein each member of the library has a charge that is less negative than the DAPRin of SEQ ID NO: 1, excluding the charge contribution of the variable antigen-binding residues.

35. A DARPin library according to claim 34, wherein each member of the library has a neutral net charge.

36. A DARPin according to claim 1, wherein each member of the library has a positive net charge.

37. The DARPin library of claim 34, 35, or 36, characterised in that X₁, X₅, X₉, X₁₄, X₁₉, X₂₁, and X₂₃ are independently selected from the list consisting of Aspartate, Glutamate, Lysine, and Arginine.

38. The DARPin library of claim 34, 35, 36, or 37, characterised in that X₂, X₇, X₈, X₁₂, X₁₃, X₁₇, X₁₈, X₂₂, and X₂₄ are independently selected from Glutamate and Lysine.

39. The DARPin library of any one of claims 34 to 38, characterised in that X₃ is selected from Isoleucine and Arginine.

40. The DARPin library of any one of claims 34 to 39, characterised in that X₄ is selected from Glycine and Arginine.

41. The DARPin library of any one of claims 34 to 40, characterised in that X₆, X₁₁, X₁₆, X₂₅ and X₂₆ are independently selected from Leucine and Arginine.

42. The DARPin library of any one of claims 33 to 41, characterised in that X₁₀, X₁₅, and X₂₀ are independently selected from Asparagine and Lysine.

43. The DARPin library of any one of claims 33 to 42, characterised in that X₃, X₄, X₂₀ and X₂₂ are not substituted

44. A method of identifying a Designed Ankyrin Repeat Protein (DARPin) capable of (i) binding an antigen; and (ii) crossing the membrane of a cell, the method comprising:
a) screening the library of any one of claims 34 to 42 for binding to the antigen by carrying out a selection using the antigen; and
b) purifying the DARPins.

45. The method of claim 44, further comprises between steps (a) and (b) carrying out a second selection using a negatively charged reagent and eluting the DARPins.

46. The method of claim 44 or 45, characterised in that the antigen is an intracellular antigen.

47. The method of any one of claims 44 to 46, characterised in that step (a) is carried out by a method selected from the list consisting of: phage display, yeast display, and ribosome display.

48. The method of any one of claims 44 to 47, characterised in that step (a) is carried out using the intracellular antigen alone.

49. The method of any one of claims 45 to 48, characterised in that the second selection is carried out using the intracellular antigen and a negatively charged reagent.

50. The method of any one of claims 45 to 48, characterised in that the negatively charged reagent is Heparin.

51. The method of any one of claims 45 to 48, characterised in that the negatively charged reagent is selected from the list consisting of DNA, a negatively charged protein, an anionic liquid, a negatively charged membrane, or a negatively charged resin.

52. The method of any one of claims 44 to 51, characterised in that purification step (b) is carried out in the presence of a salt buffer.

53. The method of claim 52, characterised in that purification step (b) is carried out in the presence of NaCl or KCl.


SEQUENCE LISTING (120 sequences)

SEQ ID NO: 1 - protein, 156 aa, Artificial Sequence
  Synthetic polypeptide
DLGKKLLEAARAGQDDEVRILMANGADVNAXDXXGXTPLHLAAXXGHLEIVEVLLKNGADVNAXDXXGXTPLHLAAXXGHLEIVEVLLKNGADVNAXDXXGXTPLHLAAXXGHLEIVEVLLKNGADVNAQDKFGKTAFDISIDNGNEDLAEILQKL

SEQ ID NO: 2 - protein, 30 aa, Artificial Sequence
  Synthetic polypeptide
DLGKKLLEAARAGQDDEVRILMANGADVNA

SEQ ID NO: 3 - protein, 33 aa, Artificial Sequence
  Synthetic polypeptide
XDXXGXTPLHLAAXXGHLEIVEVLLKNGADVNA

SEQ ID NO: 4 - protein, 27 aa, Artificial Sequence
  Synthetic peptide
QDKFGKTAFDISIDNGNEDLAEILQKL

SEQ ID NO: 5 - protein, 30 aa, Artificial Sequence; identical to SEQ ID NO: 2
  Synthetic polypeptide

SEQ ID NO: 6 - protein, 33 aa, Artificial Sequence
  Synthetic polypeptide
XDXXGXTPLHLAAXXGHLEIVEVLLKXGADVNA

SEQ ID NO: 7 - protein, 27 aa, Artificial Sequence
  Synthetic peptide
QDKFGKTAFDISIDNGNEDLAEILQKL

SEQ ID NO: 8 - protein, 156 aa, Artificial Sequence; identical to SEQ ID NO: 1
  Synthetic polypeptide

SEQ ID NO: 9 - protein, 156 aa, Artificial Sequence; identical to SEQ ID NO: 1
  Synthetic polypeptide

SEQ ID NO: 10 - protein, 156 aa, Artificial Sequence
  Synthetic polypeptide
DLGKKLLEAARAGQDDEVRILMANGADVNAXDXXGXTPLHLAAXXGHLEIVEVLLKNGADVNAXDXXGXTPLHLAAXXGHLEIVEVLLKNGADVNAXDXXGXTPLHLAAXXGHLEIVEVLLKNGADVNAQDKFGKTAFDISIENGNEDLAEILQKL

SEQ ID NO: 11 - protein, 156 aa, Artificial Sequence
  Synthetic polypeptide
DLGKKLLEAARAGQNDKVRILMANGAKVNAXDXXGXTPLHLAAXXGHRKIVKVLLKNGANVKAXDXXGXTPLHLAAXXGHLEIVKVLLKNGAKVKAXDXXGXTPLHLAAXXGHLEIVKVLLKNGAKVNAQDKFGKTAFDISIENGNEKLAKILQKL

SEQ ID NO: 12 - protein, 32 aa, Artificial Sequence
  Synthetic polypeptide
GSDLGKKLLEAARAGQDDEVRILLKAGADVNA

SEQ ID NO: 13 - protein, 32 aa, Artificial Sequence
  Synthetic polypeptide
GSDLGKKLLEAARAGQDDEVRELLKAGADVNA

SEQ ID NO: 14 - protein, 28 aa, Artificial Sequence
  Synthetic peptide
QDKSGKTPADLAADAGHEDIAEVLQKAA

SEQ ID NO: 15 - protein, 156 aa, Artificial Sequence
  Synthetic polypeptide
DLGKKLLEAARAGQDDEVRILMANGADVNAMDQYGFTPLHLAAWYGHLEIVEVLLKHGADVNAKDVHGFTPLHLAAWTGHLEIVEVLLKNGADVNARDNEGSTPLHLAALAGHLEIVEVLLKNGADVNAQDKFGKTAFDISIDNGNEDLAEILQKL

SEQ ID NO: 16 - DNA, 86 bp, Artificial Sequence
  Synthetic oligonucleotide
ccgtattgatccatcgcgttaacwtytgcacsgttcgccataagtmtacggacttyatcwtyctgcccggcacgcgcggcttccag

SEQ ID NO: 17 - DNA, 80 bp, Artificial Sequence
  Synthetic oligonucleotide
gaaaccgtggacatctttcgcwttaacwtytgcaccatgcttcaacagcacttycacaatttyamggtgaccgtaccacg

SEQ ID NO: 18 - DNA, 79 bp, Artificial Sequence
  Synthetic oligonucleotide
cgctacctcgttatcgcgcgcwttaacwtytgcaccgttcttcaacagcacttycacaatttygaggtgacccgtccac

SEQ ID NO: 19 - DNA, 81 bp, Artificial Sequence
  Synthetic oligonucleotide
ggttttgccaaacttatcctgagcwttcacwtytgcaccgttcttcaacagcacttycacaatttygaggtgacctgccag

SEQ ID NO: 20 - DNA, 70 bp, Artificial Sequence
  Synthetic oligonucleotide
gtgcggccgccagtttctgcaggatttycgctaawtyttygttgccattwtyaatggagatatcaaacgc

SEQ ID NO: 21 - protein, 156 aa, Artificial Sequence
  Synthetic polypeptide
DLGKKLLEAARAGQDDEVRILMANGADVNAADDWGNTPLHLAALEGHLEIVEVLLKYGADVNAQDLYGTTPLHLAAWVGHLEIVEVLLKNGADVNAMDVVGETPLHLAAEMGHLEIVEVLLKHGADVNAQDKFGKTAFDISIDNGNEDLAEILQKL

SEQ ID NO: 22 - protein, 156 aa, Artificial Sequence
  Synthetic polypeptide
DLGKKLLEAARAGQDDEVRILMANGADVNAADDWGNTPLHLAALEGHLEIVEVLLKYGADVNAQDLYGTTPLHLAAWVGHLEIVEVLLKNGAKVNAMDVVGETPLHLAAEMGHLKIVEVLLKHGADVNAQDKFGKTAFDISIDNGNEDLAEILQKL

SEQ ID NO: 23 - protein, 156 aa, Artificial Sequence
  Synthetic polypeptide
DLGKKLLEAARAGQDDEVRILMANGADVNAADDWGNTPLHLAALEGHLEIVEVLLKYGADVNAQDLYGTTPLHLAAWVGHLEIVKVLLKNGAKVNAMDVVGETPLHLAAEMGHRKIVKVLLKHGAKVNAQDKFGKTAFDISIENGNEKLAKILQKL

SEQ ID NO: 24 - protein, 156 aa, Artificial Sequence
  Synthetic polypeptide
DLGKKLLEAARAGQDDEVRILMANGAKVNAADDWGNTPLHLAALEGHRKIVKVLLKYGAKVNAQDLYGTTPLHLAAWVGHLEIVKVLLKNGAKVNAMDVVGETPLHLAAEMGHLEIVKVLLKHGADVNAQDKFGKTAFDISIDNGNEDLAKILQKL

SEQ ID NO: 25 - protein, 156 aa, Artificial Sequence
  Synthetic polypeptide
DLGKKLLEAARAGQNDKVRILMANGAKVNAADDWGNTPLHLAALEGHLEIVKVLLKYGANVKAQDLYGTTPLHLAAWVGHLEIVEVLLKNGANVKAMDVVGETPLHLAAEMGHLEIVKVLLKHGAKVNAQDKFGKTAFDISIENGNEKLAKILQKL

SEQ ID NO: 26 - protein, 156 aa, Artificial Sequence
  Synthetic polypeptide
DLGKKLLEAARAGQNDKVRILMANGAKVNAADDWGNTPLHLAALEGHLEIVKVLLKYGAKVNAQDLYGTTPLHLAAWVGHLEIVKVLLKNGANVKAMDVVGETPLHLAAEMGHLEIVKVLLKHGAKVNAQDKFGKTAFDISIENGNEKLAKILQKL

SEQ ID NO: 27 - protein, 156 aa, Artificial Sequence
  Synthetic polypeptide
DLGKKLLEAARAGQNDKVRILMANGAKVNAADDWGNTPLHLAALEGHLEIVEVLLKYGAKVNAQDLYGTTPLHLAAWVGHLEIVKVLLKNGAKVNAMDVVGETPLHLAAEMGHRKIVKVLLKHGAKVNAQDKFGKTAFDISIENGNEKLAKILQKL

SEQ ID NO: 28 - protein, 156 aa, Artificial Sequence
  Synthetic polypeptide
DLGKKLLEAARAGQNDKVRILMANGAKVNAADDWGNTPLHLAALEGHLEIVKVLLKYGADVNAQDLYGTTPLHLAAWVGHLEIVKVLLKNGAKVNAMDVVGETPLHLAAEMGHRKIVKVLLKHGAKVNAQDKFGKTAFDISIENGNEKLAKILQKL

SEQ ID NO: 29 - protein, 156 aa, Artificial Sequence
  Synthetic polypeptide
DLGKKLLEAARAGQNDKVRILMANGAKVNAADDWGNTPLHLAALEGHLEIVKVLLKYGADVNAQDLYGTTPLHLAAWVGHRKIVKVLLKNGANVKAMDVVGETPLHLAAEMGHLEIVKVLLKHGAKVNAQDKFGKTAFDISIENGNEKLAKILQKL

SEQ ID NO: 30 - protein, 156 aa, Artificial Sequence
  Synthetic polypeptide
DLGKKLLEAARAGQNDKVRILMANGADVKAADDWGNTPLHLAALEGHLEIVKVLLKYGANVKAQDLYGTTPLHLAAWVGHLEIVKVLLKNGAKVNAMDVVGETPLHLAAEMGHRKIVKVLLKHGAKVNAQDKFGKTAFDISIENGNEKLAKILQKL

SEQ ID NO: 31 - protein, 156 aa, Artificial Sequence
  Synthetic polypeptide
DLGKKLLEAARAGQNDKVRILMANGAKVNAADDWGNTPLHLAALEGHLEIVKVLLKYGANVKAQDLYGTTPLHLAAWVGHLEIVKVLLKNGAKVNAMDVVGETPLHLAAEMGHRKIVKVLLKHGAKVNAQDKFGKTAFDISIENGNEKLAKILQKL

SEQ ID NO: 32 - protein, 156 aa, Artificial Sequence
  Synthetic polypeptide
DLGKKLLEAARAGQNDKVRILMANGAKVNAADDWGNTPLHLAALEGHRKIVKVLLKYGANVKAQDLYGTTPLHLAAWVGHLEIVKVLLKNGAKVNAMDVVGETPLHLAAEMGHRKIVKVLLKHGAKVNAQDKFGKTAFDISIENGNEKLAKILQKL

SEQ ID NO: 33 - protein, 156 aa, Artificial Sequence
  Synthetic polypeptide
DLGKKLLEAARAGQDDEVRILMANGADVNAADTWGRTPLHLAAYHGHLEIVEVLLKHGADVNAEDIHGFTPLHLAALWGHLEIVEVLLKNGADVNAVDWYGHTPLHLAAIDGHLEIVEVLLKHGADVNAQDKFGKTAFDISIDNGNEDLAEILQKL

SEQ ID NO: 34 - protein, 156 aa, Artificial Sequence
  Synthetic polypeptide
DLGKKLLEAARAGQDDEVRILMANGADVNAADTWGRTPLHLAAYHGHLEIVEVLLKHGADVNAEDIHGFTPLHLAALWGHLEIVKVLLKNGADVNAVDWYGHTPLHLAAIDGHLEIVEVLLKHGAKVNAQDKFGKTAFDISIDNGNEKLAEILQKL

SEQ ID NO: 35 - protein, 156 aa, Artificial Sequence
  Synthetic polypeptide
DLGKKLLEAARAGQNDKVRILMANGADVNAADTWGRTPLHLAAYHGHLEIVKVLLKHGADVNAEDIHGFTPLHLAALWGHLEIVKVLLKNGADVNAVDWYGHTPLHLAAIDGHLEIVEVLLKHGADVNAQDKFGKTAFDISIENGNEKLAKILQKL

SEQ ID NO: 36 - protein, 156 aa, Artificial Sequence
  Synthetic polypeptide
DLGKKLLEAARAGQNDKVRILMANGAKVNAADTWGRTPLHLAAYHGHLEIVKVLLKHGANVKAEDIHGFTPLHLAALWGHLEIVKVLLKNGADVNAVDWYGHTPLHLAAIDGHLEIVEVLLKHGADVNAQDKFGKTAFDISIENGNEKLAKILQKL

SEQ ID NO: 37 - protein, 156 aa, Artificial Sequence
  Synthetic polypeptide
DLGKKLLEAARAGQNDKVRILMANGAKVNAADTWGRTPLHLAAYHGHLEIVKVLLKHGANVKAEDIHGFTPLHLAALWGHLEIVKVLLKNGADVNAVDWYGHTPLHLAAIDGHRKIVKVLLKHGAKVNAQDKFGKTAFDISIDNGNEDLAEILQKL

SEQ ID NO: 38 - protein, 156 aa, Artificial Sequence
  Synthetic polypeptide
DLGKKLLEAARAGQNDKVRILMANGAKVNAADTWGRTPLHLAAYHGHLEIVKVLLKHGADVNAEDIHGFTPLHLAALWGHLEIVKVLLKNGAKVNAVDWYGHTPLHLAAIDGHLEIVKVLLKHGAKVNAQDKFGKTAFDISIENGNEKLAKILQKL

SEQ ID NO: 39 - protein, 156 aa, Artificial Sequence
  Synthetic polypeptide
DLGKKLLEAARAGQNDKVRILMANGAKVNAADTWGRTPLHLAAYHGHLEIVKVLLKHGANVKAEDIHGFTPLHLAALWGHLEIVKVLLKNGAKVNAVDWYGHTPLHLAAIDGHLEIVKVLLKHGAKVNAQDKFGKTAFDISIENGNEKLAKILQKL

SEQ ID NO: 40 - protein, 156 aa, Artificial Sequence
  Synthetic polypeptide
DLGKKLLEAARAGQNDKVRILMANGAKVNAADTWGRTPLHLAAYHGHRKIVKVLLKHGANVKAEDIHGFTPLHLAALWGHLEIVKVLLKNGADVNAVDWYGHTPLHLAAIDGHLEIVKVLLKHGAKVNAQDKFGKTAFDISIENGNEKLAKILQKL

SEQ ID NO: 41 - protein, 156 aa, Artificial Sequence
  Synthetic polypeptide
DLGKKLLEAARAGQNDKVRILMANGAKVNAADTWGRTPLHLAAYHGHLEIVKVLLKHGANVKAEDIHGFTPLHLAALWGHLEIVKVLLANGAKVNAVDWYGHTPLHLAAIDGHRKIVKVLLKHGAKVNAQDKFGKTAFDISIENGNEKLAKILQKL

SEQ ID NO: 42 - protein, 156 aa, Artificial Sequence
  Synthetic polypeptide
DLGKKLLEAARAGQNDKVRILMANGAKVNAADTWGRTPLHLAAYHGHLEIVKVLLKHGANVKAEDIHGFTPLHLAALWGHRKIVKVLLKNGAKVNAVDWYGHTPLHLAAIDGHLEIVKVLLKHGAKVNAQDKFGKTAFDISIENGNEKLAKILQKL

SEQ ID NO: 43 - protein, 156 aa, Artificial Sequence
  Synthetic polypeptide
DLGKKLLEAARAGQNDKVRILMANGAKVNAADTWGRTPLHVAPYHGHLKIVKVLLKHGANVKAEDIHGFTPLHLAALWGHLKIVKVLLKNGAKVNAVDWYGHTPLHLAAIDGHLEIVKVLLKHGAKVNAQDKFGKTAFDISIENGNEKLAKILQKL

SEQ ID NO: 44 - protein, 156 aa, Artificial Sequence
  Synthetic polypeptide
DLGKKLLEAARAGQNDKVRILMANGAKVNAADTWGRTPLHLAAYHGHLEIVKVLLKHGANVNAEDIHGFTPLHLAALWGHRKIVKVLLKNGAKVNAVDWYGHTPLHLAAIDGHRKIVKVLLKHGAKVNAQDKFGKTAFDISIENGNEKLAKILQKL

SEQ ID NO: 45 - protein, 156 aa, Artificial Sequence; identical to SEQ ID NO: 15
  Synthetic polypeptide

SEQ ID NO: 46 - protein, 156 aa, Artificial Sequence
  Synthetic polypeptide
DLGKKLLEAARAGQNDKVRILMANGAKVNAMDQYGFTPLHLAAWYGHLEIVEVLLKHGADVNAKDVHGFTPLHLAAWTGHLEIVEVLLKNGADVNARDNEGSTPLHLAALAGHLEIVEVLLKNGADVNAQDKFGKTAFDISIDNGNEDLAEILQKL

SEQ ID NO: 47 - protein, 156 aa, Artificial Sequence
  Synthetic polypeptide
DLGKKLLEAARAGQDDKVRILMANGADVNAMDQYGFTPLHLAAWYGHLEIVKVLLKHGANVKAKDVHGFTPLHLAAWTGHLEIVKVLLKNGAKVNARDNEGSTPLHLAALAGHLEIVKVLLKNGAKVNAQDKFGKTAFDISIDNGNEDLAEILQKL

SEQ ID NO: 48 - protein, 156 aa, Artificial Sequence
  Synthetic polypeptide
DLGKKLLEAARAGQNDEVRILMANGAKVNAMDQYGFTPLHLAAWYGHRKIVKVLLKHGADVNAKDVHGFTPLHLAAWTGHLEIVKVLLKNGAKVNARDNEGSTPLHLAALAGHLEIVKVLLKNGADVNAQDKFGKTAFDISIENGNEKLAKILQKL

SEQ ID NO: 49 - protein, 156 aa, Artificial Sequence
  Synthetic polypeptide
DLGKKLLEAARAGQNDKVRILMANGAKVNAMDQYGFTPLHLAAWYGHLEIVKVLLKHGANVKAKDVHGFTPLHLAAWTGHLEIVKVLLKNGAKVNARDNEGSTPLHLAALAGHLEIVEVLLKNGAKVNAQDKFGKTAFDISIENGNEKLAKILQKL

SEQ ID NO: 50 - protein, 156 aa, Artificial Sequence
  Synthetic polypeptide
DLGKKLLEAARAGQNDKVRILMANGAKVNAMDQYGFTPLHLAAWYGHLEIVKVLLKHGANVKAKDVHGFTPLHLAAWTGHLEIVKVLLKNGAKVNARDNEGSTPLHLAALAGHLEIVKVLLKNGAKVNAQDKFGKTAFDISIENGNEKLAKILQKL

SEQ ID NO: 51 - protein, 156 aa, Artificial Sequence
  Synthetic polypeptide
DLGKKLLEAARAGQNDKVRILMANGAKVNAMDQYGFTPLHLAAWYGHRKIVEVLLKHGANVKAKDVHGFTPLHLAAWTGHLEIVKVLLKNGAKVNARDNEGSTPLHLAALAGHLEIVKVLLKNGAKVNAQDKFGKTAFDISIENGNEKLAKILQKL

SEQ ID NO: 52 - protein, 156 aa, Artificial Sequence
  Synthetic polypeptide
DLGKKLLEAARAGQNDKVRILMANGANVKAMDQYGFTPLHLAAWYGHRKIVKVLLKHGANVKAKDVHGFTPLHLAAWTGHLEIVEVLLKNGADVNARDNEGSTPLHLAALAGHRKIVKVLLKNGAKVNAQDKFGKTAFDISIENGNEKLAKILQKL

SEQ ID NO: 53 - protein, 156 aa, Artificial Sequence
  Synthetic polypeptide
DLGKKLLEAARAGQNDKVRILMANGAKVNAMDQYGFTPLHLAAWYGHRKIVKVLLKHGANVKAKDVHGFTPLHLAAWTGHLEIVKVLLKNGANVKARDNEGSTPLHLAALAGHLEIVKVLLKNGAKVNAQDKFGKTAFDISIENGNEKLAKILQKL

SEQ ID NO: 54 - protein, 156 aa, Artificial Sequence
  Synthetic polypeptide
DLGKKLLEAARAGQNDKVRILMANGAKVNAMDQYGFTPLHLAAWYGHRKIVKVLLKHGANVKAKDVHGFTPLHLAAWTGHLEIVKVLLKNGAKVNARDNEGSTPLHLAALAGHLEIVKVLLKNGAKVNAQDKFGKTAFDISIENGNEKLAKILQKL

SEQ ID NO: 55 - protein, 156 aa, Artificial Sequence; identical to SEQ ID NO: 54
  Synthetic polypeptide

SEQ ID NO: 56 - protein, 156 aa, Artificial Sequence
  Synthetic polypeptide
DLGKKLLEAARAGQNDKVRILMANGADVNAMDQYGFTPLHLAAWYGHRKIVKVLLKHGANVKAKDVHGFTPLHLAAWTGHRKIVKVLLKNGANVKARDNEGSTPLHLAALAGHLEIVKVLLKNGAKVNAQDKFGKTAFDISIENGNEKLAKILQKL

SEQ ID NO: 57 - protein, 156 aa, Artificial Sequence
  Synthetic polypeptide
DLGKKLLEAARAGQDDEVRILMANGADVNANDKFGRTPLHLAAIWGHLEIVEVLLKYGADVNATDSMGNTPLHLAALLGHLEIVEVLLKNGADVNALDNAGRTPLHLAADDGHLEIVEVLLKYGADVNAQDKFGKTAFDISIDNGNEDLAEILQKL

SEQ ID NO: 58 - protein, 156 aa, Artificial Sequence
  Synthetic polypeptide
DLGKKLLEAARAGQDDEVRILMANGADVNANDKFGRTPLHLAAIWGHLEIVEVLLKYGADVNATDSMGNTPLHLAALLGHLEIVEVLLKNGANVKALDNAGRTPLHLAADDGHLEIVEVLLKYGAKVNAQDKFGKTAFDISIDNGNEDLAEILQKL

SEQ ID NO: 59 - protein, 156 aa, Artificial Sequence
  Synthetic polypeptide
DLGKKLLEAARAGQDDEVRILMANGAKVNANDKFGRTPLHLAAIWGHLEIVEVLLKYGADVNATDSMGNTPLHLAALLGHLEIVEVLLKNGAKVNALDNAGRTPLHLAADDGHLEIVEVLLKYGAKVNAQDKFGKTAFDISIENGNEKLAKILQKL

SEQ ID NO: 60 - protein, 156 aa, Artificial Sequence
  Synthetic polypeptide
DLGKKLLEAARAGQNDKVRILMANGAKVNANDKFGRTPLHLAAIWGHLEIVKVLLKYGADVNATDSMGNTPLHLAALLGHLEIVKVLLKNGADVNALDNAGRTPLHLAADDGHLEIVKVLLKYGAKVNAQDKFGKTAFDISIENGNEKLAKILQKL

SEQ ID NO: 61 - protein, 156 aa, Artificial Sequence
  Synthetic polypeptide
DLGKKLLEAARAGQNDKVRILMANGADVNANDKFGRTPLHLAAIWGHRKIVKVLLKYGADVNATDSMGNTPLHLAALLGHLEIVKVLLKNGAKVNALDNAGRTPLHLAADDGHLEIVKVLLKYGADVNAQDKFGKTAFDISIENGNEKLAKILQKL

SEQ ID NO: 62 - protein, 156 aa, Artificial Sequence
  Synthetic polypeptide
DLGKKLLEAARAGQNDKVRILMANGAKVNANDKFGRTPLHLAAIWGHRKIVKVLLKYGANVKATDSMGNTPLHLAALLGHLEIVKVLLKNGADVNALDNAGRTPLHLAADDGHLEIVKVLLKYGADVNAQDKFGKTAFDISIDNGNEKLAKILQKL

SEQ ID NO: 63 - protein, 156 aa, Artificial Sequence
  Synthetic polypeptide
DLGKKLLEAARAGQNDKVRILMANGAKVNANDKFGRTPLHLAAIWGHLEIVKVLLKYGANVKATDSMGNTPLHLAALLGHLEIVKVLLKNGANVKALDNAGRTPLHLAADDGHLEIVKVLLKYGAKVNAQDKFGKTAFDISIENGNEKLAKILQKL

SEQ ID NO: 64 - protein, 156 aa, Artificial Sequence
  Synthetic polypeptide
DLGKKLLEAARAGQNDKVRILMANGAKVNANDKFGRTPLHLAAIWGHRKIVKVLLKYGAYVNATDSMGNTPLHLAALLGHLEIVKVLLKSGANVKALDNAGRTPLHLAADDGHLEIVKVLLKYGAKVNAQDKFGKTAFDISIENGNEKLAKILQKL

SEQ ID NO: 65 - protein, 156 aa, Artificial Sequence
  Synthetic polypeptide
DLGKKLLEAARAGQNDKVRILMANGAKVNANDKFGRTPLHLAAIWGHLEIVKVLLKYGANVKATDSMGNTPLHLAALLGHRKIVKVLLKNGAKVNALDNAGRTPLHLAADDGHLEIVKVLLKYGAKVNAQDKFGKTAFDISIENGNEKLAKILQKL

SEQ ID NO: 66 - protein, 156 aa, Artificial Sequence
  Synthetic polypeptide
DLGKKLLEAARAGQNDKVRILMANGAKVNANDKFGRTPLHLAAIWGHRKIVKVLLKYGANVKATDSMGNTPLHLAALLGHRKIVKVLLKNGADVNALDNAGRTPLHLAADDGHLEIVKVLLKYGAKVNAQDKFGKTAFDISIENGNEKLAKILQKL

SEQ ID NO: 67 - protein, 156 aa, Artificial Sequence
  Synthetic polypeptide
DLGKKLLEAARAGQNDKVRILMANGAKVNANDKFGRTPLHLAAIWGHRKIVKVLLKYGANVKATDSMGNTPLHLAALLGHLEIVKVLLKNGANVKALDNAGRTPLHLAADDGHRKIVKVLLKYGAKVNAQDKFGKTAFDISIENGNEKLAKILQKL

SEQ ID NO: 68 - protein, 156 aa, Artificial Sequence
  Synthetic polypeptide
DLGKKLLEAARAGQNDKVRILMANGAKVNANDKFGRTPLHLAAIWGHRKIVKVLLKYGANVKATDSMGNTPLHLAALLGHRKIVKVLLKNGAKVNALDNAGRTPLHLAADDGHRKIVKVLLKYGAKVNAQDKFGKTAFDISIENGNEKLAKILQKL

SEQ ID NO: 69 - protein, 156 aa, Artificial Sequence
  Synthetic polypeptide
DLGKKLLEAARAGQDDEVRILMANGADVNAVDAFGHTPLHLAADAGHLEIVEVLLKYGADVNAYDMNGYTPLHLAAWLGHLEIVEVLLKYGADVNATDHNGYTPLHLAAHLGHLEIVEVLLKHGADVNAQDKFGKTAFDISIDNGNEDLAEILQKL

SEQ ID NO: 70 - protein, 156 aa, Artificial Sequence
  Synthetic polypeptide
DLGKKLLEAARAGQDDEVRILMANGADVNAVDAFGHTPLHLAADAGHLEIVEVLLKYGADVNAYDMNGYTPLHLAAWLGHLEIVKVLLKYGADVNATDHNGYTPLHLAAHLGHLEIVEVLLKHGADVNAQDKFGKTAFDISIDNGNEDLAEILQKL

SEQ ID NO: 71 - protein, 156 aa, Artificial Sequence
  Synthetic polypeptide
DLGKKLLEAARAGQDDEVRILMANGANVNAVDAFGHTPLHLAADAGHLEIVEVLLKYGADVNAYDMNGYTPLHLAAWLGHLEIVEVLLKYGAEVNATDHNGYTPLHLAAHLGHLEIVEVLLKHGADVNAQDKFGKTAFDISIDNGNEKLAEILQKL

SEQ ID NO: 72 - protein, 156 aa, Artificial Sequence
  Synthetic polypeptide
DLGKKLLEAARAGQDDEVRILMANGADVNAVDAFGHTPLHLAADAGHRKIVKVLLKYGADVNAYDMNGYTPLHLAAWLGHLEIVKVLLKYGANVKATDHNGYTPLHLAAHLGHLEIVEVLLKHGADVNAQDKFGKTAFDISIDNGNEDLAEILQKL

SEQ ID NO: 73 - protein, 156 aa, Artificial Sequence
  Synthetic polypeptide
DLGKKLLEAARAGQDDEVRILMANGADVNAVDAFGHTPLHLAADAGHLEIVKVLLKYGADVNAYDMNGYTPLHLAAWLGHLEIVKVLLKYGAKVNATDHNGYTPLHLAAHLGHLEIVKVLLKHGAKVNAQDKFGKTAFDISIENGNEKLAKILQKL

SEQ ID NO: 74 - protein, 156 aa, Artificial Sequence
  Synthetic polypeptide
DLGKKLLEAARAGQNDKVRILMANGAKVNAVDAFGHTPLHLAADAGHLEIVKVLLKYGANVKAYDMNGYTPLHLAAWLGHLEIVKVLLKYGADVNATDHNGYTPLHLAAHLGHLEIVKVLLKHGADVNAQDKFGKTAFDISIENGNEKLAKILQKL

SEQ ID NO: 75 - protein, 156 aa, Artificial Sequence
  Synthetic polypeptide
DLGKKLLEAARAGQNDKVRILMANGAKVNAVDAFGHTPLHLAADAGHLEIVKVLLKYGAKVNAYDMNGYTPLHLAAWLGHLEIVKVLLKYGADVNATDHNGYTPLHLAAHLGHLEIVKVLLKHGAKVNAQDKFGKTAFDISIENGNEKLAKILQKL

SEQ ID NO: 76 - protein, 156 aa, Artificial Sequence
  Synthetic polypeptide
DLGKKLLEAARAGQNDKVRILMANGAKVNAVDAFGHTPLHLAADAGHLEIVKVLLKYGANVKAYDMNGYTPLHLAAWLGHLEIVKVLLKYGANVKATDHNGYTPLHLAAHLGHLEIVKVLLKHGAKVNAQDKFGKTAFDISIENGNEKLAKILQKL

SEQ ID NO: 77 - protein, 156 aa, Artificial Sequence
  Synthetic polypeptide
DLGKKLLEAARAGQNDKVRILMANGAKVNAVDAFGHTPLHLAADAGHRKIVKVLLKYGANVKAYDMNGYTPLHLAAWLGHLEIVKVLLKYGADVNATDHNGYTPLHLAAHLGHLEIVKVLLKHGAKVNAQDKFGKTAFDISIENGNEKLAKILQKL

SEQ ID NO: 78 - protein, 156 aa, Artificial Sequence
  Synthetic polypeptide
DLGKKLLEAARAGQNDKVRILMANGAKVNAVDAFGHTPLHLAADAGHRKIVKVLLKYGAKVNAYDMNGYTPLHLAAWLGHLEIVKVLLKYGANVKATDHNGYTPLHLAAHLGHLEIVKVLLKHGAKVNAQDKFGKTAFDISIENGNEKLAKILQKL

SEQ ID NO: 79 - protein, 156 aa, Artificial Sequence
  Synthetic polypeptide
DLGKKLLEAARAGQNDKVRILMANGAKVNAVDAFGHTPLHLAADAGHRKIVKVLLKYGANVKAYDMNGYTPLHLAAWLGHRKIVKVLLKYGADVNATDHNGYTPLHLAAHLGHLEIVKVLLKHGAKVNAQDKFGKTAFDISIENGNEKLAKILQKL

SEQ ID NO: 80 - protein, 156 aa, Artificial Sequence
  Synthetic polypeptide
DLGKKLLEAARAGQNDKVRILMANGAKVNAVDAFGHTPLHLAADAGHRKIVKVLLKYGANVKAYDMNGYTPLHLAAWLGHLEIVKVLLKYGANVKATDHNGYTPLHLAAHLGHRKIVKVLLKHGAKVNAQDKFGKTAFDISIENGNEKLAKILQKL

SEQ ID NO: 81 - protein, 156 aa, Artificial Sequence
  Synthetic polypeptide
DLGKKLLEAARAGQDDEVRILMANGADVNARDWMGHTPLHLAAFHGHLEIVEVLLKHGADVNASDWMGKTPLHLAAWIGHLEIVEVLLKYGADVNAEDAHGYTPLHLAAMYGHLEIVEVLLKNGADVNAQDKFGKTAFDISIDNGNEDLAEILQKL

SEQ ID NO: 82 - protein, 156 aa, Artificial Sequence
  Synthetic polypeptide
DLGKKLLEAARAGQDDEVRILMANGADVNARDWMGHTPLHLAAFHGHLEIVEVLLKHGADVNASDWMGKTPLHLAAWIGHLEIVEVLLKYGADVNAEDAHGYTPLHLAAMYGHLEIVEVLLKNGAKVNAQDKFGKTAFDISIENGNEKLAKILQKL

SEQ ID NO: 83 - protein, 156 aa, Artificial Sequence
  Synthetic polypeptide
DLGKKLLEAARAGQNDKVRILMANGAKVNARDWMGHTPLHLAAFHGHLEIVKVLLKHGADVNASDWMGKTPLHLAAWIGHLEIVKVLLKYGADVNAEDAHGYTPLHLAAMYGHLEIVKVLLKNGAKVNAQDKFGKTAFDISIENGNEKLAKILQKL

SEQ ID NO: 84 - protein, 156 aa, Artificial Sequence
  Synthetic polypeptide
DLGKKLLEAARAGQNDKVRILMANGAKVNARDWMGHTPLHLAAFHGHLEIVKVLLKHGANVKASDWMGKTPLHLAAWIGHLEIVKVLLKYGADVNAEDAHGYTPLHLAAMYGHLEIVKVLLKNGAKVNAQDKFGKTAFDISIENGNEKLAKILQKL

SEQ ID NO: 85 - protein, 156 aa, Artificial Sequence
  Synthetic polypeptide
DLGKKLLEAARAGQNDKVRILMANGADVNARDWMGHTPLHLAAFHGHLEIVKVLLKHGADVNASDWMGKTPLHLAAWIGHRKIVKVLLKYGADVNAEDAHGYTPLHLAAMYGHRKIVKVLLKNGAKVNAQDKFGKTAFDISIENGNEKLAKILQKL

SEQ ID NO: 86 - protein, 156 aa, Artificial Sequence
  Synthetic polypeptide
DLGKKLLEAARAGQNDKVRILMANGADVNARDWMGHTPLHLAAFHGHRKIVKVLLKHGADVNASDWMGKTPLHLAAWIGHLEIVKVLLKYGANVKAEDAHGYTPLHLAAMYGHRKIVKVLLKNGAKVNAQDKFGKTAFDISIENGNEKLAKILQKL

SEQ ID NO: 87 - protein, 156 aa, Artificial Sequence
  Synthetic polypeptide
DLGKKLLEAARAGQNDKVRILMANGAKVNARDWMGHTPLHLAAFHGHRKIVKVLLKHGADVNASDWMGKTPLHLAAWIGHLEIVKVLLKYGANVKAEDAHGYTPLHLAAMYGHRKIVKVLLKNGAKVNAQDKFGKTAFDISIENGNEKLAKILQKL

SEQ ID NO: 88 - protein, 156 aa, Artificial Sequence; identical to SEQ ID NO: 87
  Synthetic polypeptide

SEQ ID NO: 89 - protein, 156 aa, Artificial Sequence
  Synthetic polypeptide
DLGKKLLEAARAGQNDKVRILMANGAKVNARDWMGHTPLHLAAFHGHLKIVKVLLKHGADVNASDWMGKTPLHLAAWIGHRKIVKVLLKYGANVKAEDAHGYTPLHLAAMYGHLKIVKVLLKNGAKVNAQDKFGKTAFDISIENGNEKLAKILQKL

SEQ ID NO: 90 - protein, 156 aa, Artificial Sequence
  Synthetic polypeptide
DLGKKLLEAARAGQNDKVRILMANGAKVNARDWMGHTPLHLAAFHGHRKIVKVLLKHGAKVNASDWMGKTPLHLAAWIGHRKIVKVLLKYGANVKAEDAHGYTPLHLAAMYGHLEIVKVLLKNGAKVNAQDKFGKTAFDISIENGNEKLAKILQKL

SEQ ID NO: 91 - protein, 156 aa, Artificial Sequence
  Synthetic polypeptide
DLGKKLLEAARAGQDDEVRILMANGADVNARDHTGFTPLHLAAQHGHLEIVEVLLKNGADVNAVDWFGRTPLHLAAKMGHLEIVEVLLKHGADVNADDHNGRTPLHLAAWEGHLEIVEVLLKYGADVNAQDKFGKTAFDISIDNGNEDLAEILQKL

SEQ ID NO: 92 - protein, 156 aa, Artificial Sequence
  Synthetic polypeptide
DLGKKLLEAARAGQDDEVRILMANGADVNARDHTGFTPLHLAAQHGHLEIVEVLLKNGADVNAVDWFGRTPLHLAAKMGHLEIVEVLLKHGADVNADDHNGRTPLHLAAWEGHLEIVEVLLKYGAEVNAQDKFGKTAFDISIENGNEKLAKILQKL

SEQ ID NO: 93 - protein, 156 aa, Artificial Sequence
  Synthetic polypeptide
DLGKKLLEAARAGQNDKVRILMANGAKVNARDHTGFTPLHLAAQHGHLEIVEVLLKNGANVNAVDWFGRTPLHLAAKMGHLEIVEVLLKHGANVNADDHNGRTPLHLAAWEGHLEIVKVLLKYGAKVNAQDKFGKTAFDISIDNGNEKLAEILQKL

SEQ ID NO: 94 - protein, 156 aa, Artificial Sequence
  Synthetic polypeptide
DLGKKLLEAARAGQNDKVRILMANGADVNARDHTGFTPLHLAAQHGHLEIVKVLLKNGANVKAVDWFGRTPLHLAAKMGHLEIVKVLLKHGADVNADDHNGRTPLHLAAWEGHLEIVEVLLKYGAKVNAQDKFGKTAFDISIENGNEKLAKILQKL

SEQ ID NO: 95 - protein, 156 aa, Artificial Sequence
  Synthetic polypeptide
DLGKKLLEAARAGQNDKVRILMANGADVNARDHTGFTPLHLAAQHGHRKIVKVLLKNGADVNAVDWFGRTPLHLAAKMGHLEIVKVLLKHGANVKADDHNGRTPLHLAAWEGHLEIVKVLLKYGADVNAQDKFGKTAFDISIENGNEKLAKILQKL

SEQ ID NO: 96 - protein, 156 aa, Artificial Sequence
  Synthetic polypeptide
DLGKKLLEAARAGQNDKVRILMANGAKVNARDHTGFTPLHLAAQHGHLEIVKVLLKNGANVKAVDWFGRTPLHLAAKMGHRKIVEVLLKHGADVNADDHNGRTPLHLAAWEGHLEIVKVLLKYGAKVNAQDKFGKTAFDISIENGNEKLAKILQKL

SEQ ID NO: 97 - protein, 156 aa, Artificial Sequence
  Synthetic polypeptide
DLGKKLLEAARAGQNDKVRILMANGAKVNARDHTGFTPLHLAAQHGHRKIVKVLLKNGADVNAVDWFGRTPLHLAAKMGHLEIVKVLLKHGAKVNADDHNGRTPLHLAAWEGHLEIVKVLLKYGAKVNAQDKFGKTAFDISIENGNEKLAKILQKL

SEQ ID NO: 98 - protein, 156 aa, Artificial Sequence
  Synthetic polypeptide
DLGKKLLEAARAGQNDKVRILMANGANVNARDHTGFTPLHLAAQHGHLEIVKVLLKNGAKVNAVDWFGRTPLHLAAKMGHRKIVKVLLKHGANVKADDHNGRTPLHLAAWEGHLEIVKVLLKYGAKVNAQDKFGKTAFDISIENGNEKLAKILQKL

SEQ ID NO: 99 - protein, 156 aa, Artificial Sequence
  Synthetic polypeptide
DLGKKLLEAARAGQNDKVRILMANGAKVNARDHTGFTPLHLAAQHGHRKIVKVLLKNGAKVNAVDWFGRTPLHLAAKMGHLEIVKVLLKHGANVKADDHNGRTPLHLAAWEGHLEIVKVLLKYGAKVNAQDKFGKTAFDISIENGNEKLAKILQKL

SEQ ID NO: 100 - protein, 156 aa, Artificial Sequence
  Synthetic polypeptide
DLGKKLLEAARAGQNDKVRILMANGAKVNARDHTGFTPLHLAAQHGHRKIVKVLLKNGANVKAVDWFGRTPLHLAAKMGHRKIVEVLLKHGANVKADDHNGRTPLHLAAWEGHRKIVKVLLKYGADVNAQDKFGKTAFDISIENGNEKLAKILQKL

SEQ ID NO: 101 - protein, 156 aa, Artificial Sequence
  Synthetic polypeptide
DLGKKLLEAARAGQNDKVRILMANGAKVNARDHTGFTPLHLAAQHGHRKIVKVLLKNGAKVNAVDWFGRTPLHLAAKMGHLEIVKVLLKHGANVKADDHNGRTPLHLAAWEGHRKIVKVLLKYGAKVNAQDKFGKTAFDISVENGNEKLAKILQKL

SEQ ID NO: 102 - protein, 156 aa, Artificial Sequence
  Synthetic polypeptide
DLGKKLLEAARAGQNDKVRILMANGAKVNARDHTGFTPLHLAAQHGHRKIVKVLLKNGAKVNAVDWFGRTPLHLAAKMGHRKIVKVLLKHGADVNADDHNGRTPLHLAAWEGHRKIVKVLLKYGAKVNAQDKFGKTAFDISIENGNEKLAKILQKL

SEQ ID NO: 103 - protein, 156 aa, Artificial Sequence
  Synthetic polypeptide
DLGKKLLEAARAGQDDEVRILMANGADVNAXDXXGXTPLHLAAXXGHLEIVEVLLKXGADVNAXDXXGXTPLHLAAXXGHLEIVEVLLKXGADVNAXDXXGXTPLHLAAXXGHLEIVEVLLKXGADVNAQDKFGKTAFDISIDNGNEDLAEILQKL

SEQ ID NO: 104 - protein, 156 aa, Artificial Sequence
  Synthetic polypeptide
DLGKKLLEAARAGQNDKVRILMANGAKVNAXDXXGXTPLHLAAXXGHRKIVKVLLKXGANVKAXDXXGXTPLHLAAXXGHRKIVKVLLKXGANVKAXDXXGXTPLHLAAXXGHRKIVKVLLKXGAKVNAQDKFGKTAFDISIENGNEKLAKILQKL

SEQ ID NO: 105 - DNA, 59 bp, Artificial Sequence
  Synthetic oligonucleotide
gcgttaacttttgcaccgttcgccataagtatacggactttatcattctgcccggcacg

SEQ ID NO: 106 - DNA, 30 bp, Artificial Sequence
  Synthetic oligonucleotide
caacagcactttcacaattttacggtgacc

SEQ ID NO: 107 - DNA, 18 bp, Artificial Sequence
  Synthetic oligonucleotide
cgctttaacatttgcacc

SEQ ID NO: 108 - DNA, 22 bp, Artificial Sequence
  Synthetic oligonucleotide
caacagcactttcacaatttcg

SEQ ID NO: 109 - DNA, 22 bp, Artificial Sequence
  Synthetic oligonucleotide
cctgagcattcacttttgcacc

SEQ ID NO: 110 - DNA, 48 bp, Artificial Sequence
  Synthetic oligonucleotide
ctgcaggattttcgctaatttttcgttgccattttcaatggagatatc

SEQ ID NO: 111 - protein, 19 aa, Artificial Sequence
  Synthetic peptide
RAGQNDKVRILMANGAKVN

SEQ ID NO: 112 - protein, 10 aa, Artificial Sequence
  Synthetic peptide
GHRKIVKVLL

SEQ ID NO: 113 - protein, 6 aa, Artificial Sequence
  Synthetic peptide
GANVKA

SEQ ID NO: 114 - protein, 7 aa, Artificial Sequence
  Synthetic peptide
EIVKVLL

SEQ ID NO: 115 - protein, 7 aa, Artificial Sequence
  Synthetic peptide
GAKVNAQ

SEQ ID NO: 116 - protein, 16 aa, Artificial Sequence
  Synthetic peptide
DISIENGNEKLAKILQ

SEQ ID NO: 117 - protein, 6 aa, Artificial Sequence
  Synthetic 6xHis tag
HHHHHH

SEQ ID NO: 118 - protein, 30 aa, Artificial Sequence; identical to SEQ ID NO: 2
  Synthetic polypeptide

SEQ ID NO: 119 - protein, 33 aa, Artificial Sequence; identical to SEQ ID NO: 3
  Synthetic polypeptide

SEQ ID NO: 120 - protein, 27 aa, Artificial Sequence
  Synthetic peptide
QDKFGKTAFDISIDNGNEDLAEILQKL